SEQUENCE LISTING (13 sequences)

SEQ ID NO: 1 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 771 bp, synthetic construct
  source 1..771: mol_type = other DNA
ccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcc

SEQ ID NO: 3 - DNA, 270 bp, synthetic construct
  source 1..270: mol_type = other DNA
ccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 4 - DNA, 98 bp, synthetic construct
  source 1..98: mol_type = other DNA
gtaagtttagtcttttttgtcttttatttcaggtcccggatccggtggtggtgcaaatcaaagaactgctcctcagtcgatgttgcctttacttctag

SEQ ID NO: 5 - DNA, 3591 bp, synthetic construct
  source 1..3591: mol_type = other DNA
atgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggatcttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatttag

SEQ ID NO: 6 - DNA, 82 bp, synthetic construct
  source 1..82: mol_type = other DNA
aacttgtttattgcagcttataatggttacaaataaagcaatagcatcacaaatttcacaaataaagcatttttttcactgc

SEQ ID NO: 7 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 270 bp, synthetic construct
  source 1..270: mol_type = other DNA
ttctgagttctctaaggtccctcactcccaactcagacccaagtcctgtcaattcccattcagtgtctgatctccttcttctcaccttccccatcttccatttgacccaagcttcctgagcactcctcccattcccctttttggagtcctcctcctctcccagaacccagtaataagtgggctcctccctggcctggacccccatggtaaccctataaggcgaggcagctgccatctgaggcagggaggggctggtgtgggaggctaagg

SEQ ID NO: 9 - DNA, 280 bp, synthetic construct
  source 1..280: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctac

SEQ ID NO: 10 - DNA, 5283 bp, synthetic construct
  source 1..5283: mol_type = other DNA
ccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccgtaagtttagtcttttttgtcttttatttcaggtcccggatccggtggtggtgcaaatcaaagaactgctcctcagtcgatgttgcctttacttctagatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggatcttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatttagaacttgtttattgcagcttataatggttacaaataaagcaatagcatcacaaatttcacaaataaagcatttttttcactgc

SEQ ID NO: 11 - DNA, 4714 bp, synthetic construct
  source 1..4714: mol_type = other DNA
ttctgagttctctaaggtccctcactcccaactcagacccaagtcctgtcaattcccattcagtgtctgatctccttcttctcaccttccccatcttccatttgacccaagcttcctgagcactcctcccattcccctttttggagtcctcctcctctcccagaacccagtaataagtgggctcctccctggcctggacccccatggtaaccctataaggcgaggcagctgccatctgaggcagggaggggctggtgtgggaggctaaggccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggatcttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatttagaacttgtttattgcagcttataatggttacaaataaagcaatagcatcacaaatttcacaaataaagcatttttttcactgc

SEQ ID NO: 12 - DNA, 4730 bp, synthetic construct
  source 1..4730: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcaagctagcgtacctcgcgaatgcatctagattcgcgacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggaggtaagtttagtctttttgtcttttatttcaggtcccggatccggtggtggtgcaaatcaaagaactgctcctcagtggatgttgcctttacttctagatgctttggtgggaagaagtagaggactgttatgaaagagaagatgttcaaaagaaaacattcacaaaatgggtaaatgcacaattttctaagtttgggaagcagcatattgagaacctcttcagtgacctacaggatgggaggcgcctcctagacctcctcgaaggcctgacagggcaaaaactgccaaaagaaaaaggatccacaagagttcatgccctgaacaatgtcaacaaggcactgcgggttttgcagaacaataatgttgatttagtgaatattggaagtactgacatcgtagatggaaatcataaactgactcttggtttgatttggaatataatcctccactggcaggtcaaaaatgtaatgaaaaatatcatggctggattgcaacaaaccaacagtgaaaagattctcctgagctgggtccgacaatcaactcgtaattatccacaggttaatgtaatcaacttcaccaccagctggtctgatggcctggctttgaatgctctcatccatagtcataggccagacctatttgactggaatagtgtggtttgccagcagtcagccacacaacgactggaacatgcattcaacatcgccagatatcaattaggcatagagaaactactcgatcctgaagatgttgataccacctatccagataagaagtccatcttaatgtacatcacatcactcttccaagttttgcctcaacaagtgagcattgaagccatccaggaagtggaaatgttgccaaggccacctaaagtgactaaagaagaacattttcagttacatcatcaaatgcactattctcaacagatcacggtcagtctagcacagggatatgagagaacttcttcccctaagcctcgattcaagagctatgcctacacacaggctgcttatgtcaccacctctgaccctacacggagcccatttccttcacagcatttggaagctcctgaagacaagtcatttggcagttcattgatggagagtgaagtaaacctggaccgttatcaaacagctttagaagaagtattatcgtggcttctttctgctgaggacacattgcaagcacaaggagagatttctaatgatgtggaagtggtgaaagaccagtttcatactcatgaggggtacatgatggatttgacagcccatcagggccgggttggtaatattctacaattgggaagtaagctgattggaacaggaaaattatcagaagatgaagaaactgaagtacaagagcagatgaatctcctaaattcaagatgggaatgcctcagggtagctagcatggaaaaacaaagcaatttacatagagttttaatggatctccagaatcagaaactgaaagagttgaatgactggctaacaaaaacagaagaaagaacaaggaaaatggaggaagagcctcttggacctgatcttgaagacctaaaacgccaagtacaacaacataaggtgcttcaagaagatctagaacaagaacaagtcagggtcaattctctcactcacatggtggtggtagttgatgaatctagtggagatcacgcaactgctgctttggaagaacaacttaaggtattgggagatcgatgggcaaacatctgtagatggacagaagaccgctgggttcttttacaagacatccttctcaaatggcaacgtcttactgaagaacagtgcctttttagtgcatggctttcagaaaaagaagatgcagtgaacaagattcacacaactggctttaaagatcaaaatgaaatgttatcaagtcttcaaaaactggccgttttaaaagcggatctagaaaagaaaaagcaatccatgggcaaactgtattcactcaaacaagatcttctttcaacactgaagaataagtcagtgacccagaagacggaagcatggctggataactttgcccggtgttgggataatttagtccaaaaacttgaaaagagtacagcacagatttcacaggctgtcaccaccactcagccatcactaacacagacaactgtaatggaaacagtaactacggtgaccacaagggaacagatcctggtaaagcatgctcaagaggaacttccaccaccacctccccaaaagaagaggcagattactgtggatcttgaaagactccaggaacttcaagaggccacggatgagctggacctcaagctgcgccaagctgaggtgatcaagggatcctggcagcccgtgggcgatctcctcattgactctctccaagatcacctcgagaaagtcaaggcacttcgaggagaaattgcgcctctgaaagagaacgtgagccacgtcaatgaccttgctcgccagcttaccactttgggcattcagctctcaccgtataacctcagcactctggaagacctgaacaccagatggaagcttctgcaggtggccgtcgaggaccgagtcaggcagctgcatgaagcccacagggactttggtccagcatctcagcactttctttccacgtctgtccagggtccctgggagagagccatctcgccaaacaaagtgccctactatatcaaccacgagactcaaacaacttgctgggaccatcccaaaatgacagagctctaccagtctttagctgacctgaataatgtcagattctcagcttataggactgccatgaaactccgaagactgcagaaggccctttgcttggatctcttgagcctgtcagctgcatgtgatgccttggaccagcacaacctcaagcaaaatgaccagcccatggatatcctgcagattattaattgtttgaccactatttatgaccgcctggagcaagagcacaacaatttggtcaacgtccctctctgcgtggatatgtgtctgaactggctgctgaatgtttatgatacgggacgaacagggaggatccgtgtcctgtcttttaaaactggcatcatttccctgtgtaaagcacatttggaagacaagtacagataccttttcaagcaagtggcaagttcaacaggattttgtgaccagcgcaggctgggcctccttctgcatgattctatccaaattccaagacagttgggtgaagttgcatcctttgggggcagtaacattgagccaagtgtccggagctgcttccaatttgctaataataagccagagatcgaagcggccctcttcctagactggatgagactggaaccccagtccatggtgtggctgcccgtcctgcacagagtggctgctgcagaaactgccaagcatcaggccaaatgtaacatctgcaaagagtgtccaatcattggattcaggtacaggagtctaaagcactttaattatgacatctgccaaagctgctttttttctggtcgagttgcaaaaggccataaaatgcactatcccatggtggaatattgcactccgactacatcaggagaagatgttcgagactttgccaaggtactaaaaaacaaatttcgaaccaaaaggtattttgcgaagcatccccgaatgggctacctgccagtgcagactgtcttagagggggacaacatggaaactgacacaatttagaacttgtttattgcagcttataatggttacaaataaagcaatagcatcacaaatttcacaaataaagcatttttttcactgctcgcgaatcggatcccctcgagttctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - DNA, 8112 bp, synthetic construct
  source 1..8112: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaaggaaaatgaagtgaagttcctatactttctagagaataggaacttctatagtgagtcgaataagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaacctttttatttgggggagagggaagtcatgaaaaaactaacctttgaaattcgatctccagcacatcagcaaaacgctattcacgcagtacagcaaatccttccagacccaaccaaaccaatcgtagtaaccattcaggaacgcaaccgcagcttagaccaaaacaggaagctatgggcctgcttaggtgacgtctctcgtcaggttgaatggcatggtcgctggctggatgcagaaagctggaagtgtgtgtttaccgcagcattaaagcagcaggatgttgttcctaaccttgccgggaatggctttgtggtaataggccagtcaaccagcaggatgcgtgtaggcgaatttgcggagctattagagcttatacaggcattcggtacagagcgtggcgttaagtggtcagacgaagcgagactggctctggagtggaaagcgagatggggagacagggctgcatgataaatgtcgttagtttctccggtggcaggacgtcagcatatttgctctggctaatggagcaaaagcgacgggcaggtaaagacgtgcattacgttttcatggatacaggttgtgaacatccaatgacatatcggtttgtcagggaagttgtgaagttctgggatataccgctcaccgtattgcaggttgatatcaacccggagcttggacagccaaatggttatacggtatgggaaccaaaggatattcagacgcgaatgcctgttctgaagccatttatcgatatggtaaagaaatatggcactccatacgtcggcggcgcgttctgcactgacagattaaaactcgttcccttcaccaaatactgtgatgaccatttcgggcgagggaattacaccacgtggattggcatcagagctgatgaaccgaagcggctaaagccaaagcctggaatcagatatcttgctgaactgtcagactttgagaaggaagatatcctcgcatggtggaagcaacaaccattcgatttgcaaataccggaacatctcggtaactgcatattctgcattaaaaaatcaacgcaaaaaatcggacttgcctgcaaagatgaggagggattgcagcgtgtttttaatgaggtcatcacgggatcccatgtgcgtgacggacatcgggaaacgccaaaggagattatgtaccgaggaagaatgtcgctggacggtatcgcgaaaatgtattcagaaaatgattatcaagccctgtatcaggacatggtacgagctaaaagattcgataccggctcttgttctgagtcatgcgaaatatttggagggcagcttgatttcgacttcgggagggaagctgcatgatgcgatgttatcggtgcggtgaatgcaaagaagataaccgcttccgaccaaatcaaccttactggaatcgatggtgtctccggtgtgaaagaacaccaacaggggtgttaccactaccgcaggaaaaggaggacgtgtggcgagacagcgacgaagtatcaccgacataatctgcgaaaactgcaaataccttccaacgaaacgcaccagaaataaacccaagccaatcccaaaagaatctgacgtaaaaaccttcaactacacggctcacctgtgggatatccggtggctaagacgtcgtgcgaggaaaacaaggtgattgaccaaaatcgaagttacgaacaagaaagcgtcgagcgagctttaacgtgcgctaactgcggtcagaagctgcatgtgctggaagttcacgtgtgtgagcactgctgcgcagaactgatgagcgatccgaatagctcgatgcacgaggaagaagatgatggctaaaccagcgcgaagacgatgtaaaaacgatgaatgccgggaatggtttcaccctgcattcgctaatcagtggtggtgctctccagagtgtggaaccaagatagcactcgaacgacgaagtaaagaacgcgaaaaagcggaaaaagcagcagagaagaaacgacgacgagaggagcagaaacagaaagataaacttaagattcgaaaactcgccttaaagccccgcagttactggattaaacaagcccaacaagccgtaaacgccttcatcagagaaagagaccgcgacttaccatgtatctcgtgcggaacgctcacgtctgctcagtgggatgccggacattaccggacaactgctgcggcacctcaactccgatttaatgaacgcaatattcacaagcaatgcgtggtgtgcaaccagcacaaaagcggaaatctcgttccgtatcgcgtcgaactgattagccgcatcgggcaggaagcagtagacgaaatcgaatcaaaccataaccgccatcgctggactatcgaagagtgcaaggcgatcaaggcagagtaccaacagaaactcaaagacctgcgaaatagcagaagtgaggccgcatgacgttctcagtaaaaaccattccagacatgctcgttgaagcatacggaaatcagacagaagtagcacgcagactgaaatgtagtcgcggtacggtcagaaaatacgttgatgataaagacgggaaaatgcacgccatcgtcaacgacgttctcatggttcatcgcggatggagtgaaagagatgcgctattacgaaaaaattgatggcagcaaataccgaaatatttgggtagttggcgatctgcacggatgctacacgaacctgatgaacaaactggatacgattggattcgacaacaaaaaagacctgcttatctcggtgggcgatttggttgatcgtggtgcagagaacgttgaatgcctggaattaatcacattcccctggttcagagctgtacgtggaaaccatgagcaaatgatgattgatggcttatcagagcgtggaaacgttaatcactggctgcttaatggcggtggctggttctttaatctcgattacgacaaagaaattctggctaaagctcttgcccataaagcagatgaacttccgttaatcatcgaactggtgagcaaagataaaaaatatgttatctgccacgccgattatccctttgacgaatacgagtttggaaagccagttgatcatcagcaggtaatctggaaccgcgaacgaatcagcaactcacaaaacgggatcgtgaaagaaatcaaaggcgcggacacgttcatctttggtcatacgccagcagtgaaaccactcaagtttgccaaccaaatgtatatcgataccggcgcagtgttctgcggaaacctaacattgattcaggtacagggagaaggcgcatgagactcgaaagcgtagctaaatttcattcgccaaaaagcccgatgatgagcgactcaccacgggccacggcttctgactctctttccggtactgatgtgatggctgctatggggatggcgcaatcacaagccggattcggtatggctgcattctgcggtaagcacgaactcagccagaacgacaaacaaaaggctatcaactatctgatgcaatttgcacacaaggtatcggggaaataccgtggtgtggcaaagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctggttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttattcgactctatagtgaagttcctattctctagaaagtataggaacttctgaagtggggtcgacttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttcct

(19) United States
(12) Patent Application Publication
(10) Pub. No.: US 2025/0041450 A1
(43) Pub. Date: Feb. 6, 2025

(54) ADENO-ASSOCIATED VIRUS PARTICLES AND METHODS OF USE THEREOF

(71) Applicant: Insmed Incorporated, Bridgewater, NJ (US)

(72) Inventors: Brian KASPAR, New Albany, OH (US); Allan KASPAR, Carlsbad, CA (US); Gretchen THOMSEN, San Diego, CA (US)

(21) Appl. No.: 18/294,792

(22) PCT Filed: Aug. 5, 2022
(86) PCT No.: PCT/US2022/074622

§ 371 Date: Feb. 2, 2024

Related U.S. Application Data
(60) Provisional application No. 63/239,881, filed on Sep. 1, 2021.
(60) Provisional application No. 63/229,936, filed on Aug. 5, 2021.

(51) Int. Cl. A61K 48/00; A61K 9/00; A61K 38/17; A61P 21/00; C12N 15/86

(52) U.S. Cl. CPC A61K 48/0058; A61K 9/0085; A61K 38/1719; A61K 48/0075; A61P 21/00; C12N 15/86; C12N 2750/14143; C12N 2830/50

(57) ABSTRACT

The invention provides intrathecal compositions comprising AAV particles, and their use for treating monogenic muscle disorders such as dystrophinopathies, including Duchenne muscular dystrophy.

### CROSS REFERENCE TO RELATED APPLICATIONS

This application claims priority from U.S. Provisional Application Ser. No. 63/229,936, filed on Aug. 5, 2021, and U.S. Provisional Application No. 63/239,881, filed on Sep. 1, 2021, the content of each of which is incorporated herein by reference in their entireties.

### FIELD OF THE INVENTION

The invention generally relates to adeno-associated virus (AAV) particles for delivering a micro-dystrophin transgene, methods of producing the AAV particles, cells producing the AAV particles, and methods of using the AAV particles for the delivery of a micro-dystrophin transgene to skeletal and/or cardiac muscle for the treatment of dystrophinopathies, for example, Duchenne muscular dystrophy.

### INCORPORATION OF THE SEQUENCE LISTING

The Sequence Listing associated with this application is provided in XML format in lieu of a paper copy and is hereby incorporated by reference into the specification. The name of the XML file containing the Sequence Listing is INMD_166_02WO_SeqList_ST26.xml. The XML file is approximately 40,532 bytes, was created on Aug. 3, 2022, and is being submitted electronically via USPTO Patent Center.

### BACKGROUND OF THE INVENTION

Duchenne muscular dystrophy (DMD) is inherited in an X-linked recessive pattern and is caused by a genetic mutation that prevents the body from producing dystrophin, a protein that muscles require to work properly. DMD is characterized in part by progressive muscle degeneration. As the disease progresses, initially affecting muscles in the thigh, pelvis, and arms, DMD eventually affects all voluntary muscles and involves the heart and breathing muscles in later stages. In Europe and North America, the prevalence of DMD is approximately 1 in 3,600 male births. DMD is the most common childhood onset form of muscular dystrophy and affects males almost exclusively. There is no known cure for DMD and current standards of treatment are primarily aimed at management of symptoms including: steroids, immunosuppressants, anticonvulsants, braces, corrective surgery, and assisted ventilation. Aggressive management of dilated cardiomyopathy associated with DMD includes anti-congestive medications and cardiac transplantation in severe cases.

Gene therapy is a rapidly accelerating therapeutic approach wherein nucleic acids are delivered to cells harboring mutated or non-functional genes to correct the defect in the mutated cells. In certain gene therapies, nucleic acids are packaged within adeno-associated viruses (AAV) that deliver the nucleic acids to the cells. Once inside the cell nucleus, the nucleic acid then directs appropriate protein production and the virus is safely degraded. Gene therapies for the treatment of DMD have been proposed but require delivery of high systemic viral titers leading to patient toxicity, and have high manufacturing costs due to the large quantities of virus required per patient.

Delivery of micro-dystrophin (μDys), modified but functional shortened dystrophin nucleic acid sequences, in animal models and humans has been reported to promote muscle function. μDys transgenes are designed to encode various combinations of the unique functional domains of the 427 kDa dystrophin protein. μDys sequences, generally less than 5 kilobases in length, have previously been tested using AAV to deliver μDys transgenes in the murine model of DMD using the mdx mouse, the most widely used animal model for DMD research. The mutation in the mdx mouse is a nonsense point mutation (C-to-T transition) in exon 23 that aborted full-length dystrophin expression (Sicinski et al. (1989) Science 244, pp. 1578-1580, incorporated by reference herein in its entirety). Despite the promise that delivery of μDys has shown, novel therapies are needed for the treatment of DMD. The present invention addresses this and other needs.

### BRIEF SUMMARY OF THE INVENTION

The present invention relates in part to adeno-associated virus (AAV) particles comprising a capsid that packages (i.e., encapsidates) a micro-dystrophin (μDys) transgene and methods for treating various dystrophinopathies with the same, for example, by intrathecal administration. In one embodiment, the μDys transgene encodes a μDys polypeptide comprising (i) an N-terminal region (NTD) comprising an actin binding site, (ii) a domain comprising three hinge regions and four spectrin repeats, and (iii) a cysteine rich domain. The μDys transgene, in one embodiment, comprises the nucleic acid sequence set forth in SEQ ID NO:5.

In one aspect, an AAV particle is provided, comprising a capsid encapsidating a vector genome. The vector genome, in one embodiment, comprises from 5′ to 3′: 5′ inverted terminal repeat (ITR); a promoter; a μDys transgene, an SV40 poly (A) tail; and a 3′ ITR. The μDys transgene, in one embodiment, encodes a polypeptide comprising (i) an N-terminal region (NTD) comprising an actin binding site, (ii) a central rod domain comprising from two to four hinge regions and from four to six spectrin repeats, and (iii) a cysteine rich domain. In a further embodiment, the μDys transgene encodes a μDys polypeptide comprising an NTD, hinge regions 1, 2 and 4, and spectrin repeats 1, 2, 3 and 24, and a cysteine rich domain. In even a further embodiment, the μDys transgene comprises the nucleic acid sequence set forth in SEQ ID NO:5. The AAV particle, in one embodiment, is an AAV9 particle, and is present in an effective amount in an intrathecal composition. The effective amount of the AAV particle, in one embodiment, comprises about 90% or less vector genomes than the effective vector genome amount of an intravenous (IV) composition comprising an AAV particle encapsidating a μDys transgene, e.g., the same μDys transgene that is present in the intrathecal composition.

In one embodiment, the vector genome further comprises an SV40 intron 5′ (upstream) of the μDys transgene and 3′ (downstream) of the promoter. In another embodiment, the vector genome further comprises an enhancer is 3′(downstream) of the 5′ ITR and 5′ (upstream) of the promoter.

The promoter, in one embodiment, is an MHCK7 or chicken j-actin hybrid promoter.

In a preferred embodiment, the AAV particle is an AAV9 particle, i.e., the AAV particle comprises one or more AAV9 capsid proteins. The AAV9 particle's capsid, in one embodiment, consists of AAV9 capsid proteins. In yet another embodiment, the AAV particle is an AAVrh74 particle.

In some embodiments, a recombinant AAV vector genome of the present invention comprises from 5′ to 3′: a 5′ ITR; an SK-CRM4 enhancer; a promoter; a μDys transgene; an SV40 poly (A) tail; and a 3′ ITR. In some embodiments, the SK-CRM4 enhancer has a sequence comprising or consisting of SEQ ID NO:8. In some embodiments, the μDys coding sequence encodes a μDys protein comprising an actin-binding domain and at least four spectrin repeats, e.g., from four to six spectrin repeats. In some embodiments, the μDys transgene comprises or consists of the nucleic acid sequence of SEQ ID NO:5.

In some embodiments, the encapsidated vector genome of the present invention comprises a 5′ AAV2 ITR and a 3′ AAV2 ITR. In some embodiments, the 5′ AAV2 ITR has a sequence comprising or consisting of SEQ ID NO:1. In some embodiments, the 3′ AAV2 ITR has a sequence comprising or consisting of SEQ ID NO:7.

In some embodiments, encapsidated vector genome comprises an MHCK7 promoter. In some embodiments, the MHCK7 promoter has a sequence comprising or consisting of SEQ ID NO:2. In another embodiment, the promoter is a chicken β-actin hybrid promoter. In a further embodiment, the chicken β-actin hybrid promoter has the nucleic acid sequence set forth in SEQ ID NO:3.

In some embodiments, the encapsidated vector genome of the present invention comprises an SV40 intron having a sequence comprising or consisting of SEQ ID NO:4.

In some embodiments, the encapsidated vector genome comprises a SV40 poly(A) tail having a sequence comprising or consisting of SEQ ID NO:6.

In some embodiments, the capsid of the AAV particle comprises one or more AAV1, AAV2, AAV3, AAV4, AAV5, AAV6, AAV7, AAVrh.74, AAV8, AAV9, AAV10, AAV11, AAV12, or AAV13 capsid proteins. In a preferred embodiment, the capsid is an AAV9 capsid and the AAV9 capsid consists of AAV9 capsid proteins.

In another aspect, the present invention relates to a method of treating a dystrophinopathy in a subject in need thereof, comprising, intrathecally administering to the subject in a single dose, a composition comprising an effective amount of one of the AAV particles encapsidating a vector genome comprising a μDys transgene, as further described herein. In one embodiment, the dystrophinopathy is Duchenne muscular dystrophy (DMD), Becker muscular dystrophy, or DMD-associated dilated cardiomyopathy (DCM). In even a further embodiment, the dystrophinopathy is DMD. The effective amount of the AAV particle, in a further embodiment, comprises about 90% or less vector genomes than the effective amount of a counterpart IV composition (e.g, an intravenously administered composition) comprising an AAV particle encapsidating a vector genome comprising a μDys transgene, e.g., the same μDys transgene that is present in the intrathecally administered composition.

In one embodiment, the subject is administered the composition when in the Trendelenburg position. In a further embodiment, administration is in the absence of a non-ionic, low-osmolar contrast agent.

In one embodiment of the method of treating a dystrophinopathy described herein, the effective dose of the intrathecally administered AAV particle provides a greater therapeutic response than the identical dose of an intravenously administered AAV particle encapsidating a vector genome comprising a μDys transgene, e.g., the same μDys transgene that is present in the intrathecally administered AAV particle. The therapeutic response, in one embodiment, is an increase from baseline on the North Star Ambulatory Assessment (NSAA).

In another embodiment of the method of treating a dystrophinopathy described herein, intrathecal administration of an effective amount of the AAV particle encapsidating a vector genome comprising a μDys transgene, results in a decreased number of side effects, or a reduced severity of one or more side effects in the subject, compared to the number of side effects, or severity of side effects experienced by a second subject, when the second subject is intravenously administered an effective amount of a counterpart AAV particle encapsidating a vector genome comprising a μDys transgene. In a further embodiment, the dystrophinopathy is DMD. In even a further embodiment, the AAV particle is an AAV9 particle.

In even another embodiment of the method of treating a dystrophinopathy, the effective amount of the intrathecally administered AAV particle provides greater μDys transgene expression in skeletal and/or cardiac muscle, compared to the amount of μDys transgene expression in liver tissue. In a further embodiment, the dystrophinopathy is DMD. In even a further embodiment, the AAV particle is an AAV9 particle. In yet even a further embodiment, the AAV particle encapsidates a μDys transgene having the nucleic acid sequence set forth in SEQ ID NO:5.

In another aspect of the present invention, a method for preferentially delivering a μDys transgene to skeletal and/or cardiac muscle of a subject is provided. The method entails intrathecally administering to a subject in a single dose, a composition comprising an effective amount of an AAV9 particle comprising an AAV9 capsid and a vector genome comprising a μDys transgene, encapsidated by the AAV9 capsid. The encapsidated genome comprises from 5′ to 3′: a 5′ ITR; a promoter; a μDys transgene; an SV40 poly (A) tail; and a 3′ ITR. Subsequent to administration, the μDys transgene is expressed at higher levels in the skeletal and/or cardiac muscle of the subject, compared to the transgene expression in liver tissue of the subject.

In some embodiments of a method described herein, expression of a μDys transgene delivered by an AAV particle described herein in a subject is significantly less in liver tissue of the subject as compared to skeletal and/or cardiac muscle of the subject. In a further embodiment, μDys transgene expression is at least about 10%, at least about 20%, at least about 30%, at least about 40%, at least about 50%, at least about 60%, at least about 70% or at least about 80% greater in the skeletal and/or cardiac muscle of the subject compared to the amount of μDys transgene expression in the liver tissue. In another embodiment, μDys transgene expression is at least about 10%, at least about 20%, at least about 30%, at least about 40%, at least about 50%, at least about 60%, at least about 70% or at least about 80% less in the liver of the subject compared to the amount of μDys transgene expression in skeletal and/or cardiac muscle.

### BRIEF DESCRIPTION OF THE DRAWINGS

FIG. 1A shows a schematic illustration of an exemplary μDys encoding gene construct (INS1201).

FIG. 1B shows a schematic illustration of an alternative μDys encoding gene construct (INS1212).

FIG. 1C shows an agarose gel electrophoresis of the INS1201 gene construct cloned into the psZ01 vector backbone (pSZ01-INS1201) restriction digested with HindIII/BsaI, and SmaI (lanes 1 and 2, respectively), and the INS1212 gene construct cloned into the psZ01 vector backbone (pSZ01-INS1212) restriction digested with HindIII/BsaI, and SmaI (lanes 3 and 4, respectively)

FIG. 2 shows a silver-stained SDS polyacrylamide gel electrophoresis (PAGE) of 1 μl of INS1201-AAV9 preparation (lane 1), 1 μl of INS1212-AAV9 preparation (lane 2), and 0.5 μl, 1 μl, 2 μl, and 4 μl of a 1×10¹³ vg/ml AAV2 standard (lanes 3, 4, 5, and 6, respectively).

FIG. 3A shows gastrocnemius muscle sections obtained from mdx mice 21 days post intramuscular injection with 2.7×10¹¹ vg of INS1201-AAV9 (iii) or INS1212-AAV9 (iv) and immunofluorescently stained for dystrophin. Gastrocnemius sections obtained from a non-injected mdx mouse (i) and wildtype C57/Bl mouse (ii) and immunofluorescently stained for dystrophin are shown for comparison.

FIG. 3B shows a gastrocnemius muscle section obtained from an mdx mouse 21 days post intramuscular injection with 2.7×10¹¹ vector genomes (vg) of INS1212-AAV9 and stained with DAPI (i) and for dystrophin (ii). Merged image shown in (iii).

FIG. 4A shows gastrocnemius (i), tibialis anterior (ii), quadriceps (iii), gluteus (iv), triceps (v), diaphragm (vi), heart (vii), and liver (viii) sections obtained from an mdx mouse 21 days post intracerebroventricular (ICV) injection with 2.7×10¹¹ vg of INS1201-AAV9 and immunofluorescently stained for dystrophin.

FIG. 4B shows gastroenemius (i), tibialis anterior (ii), quadriceps (iii), gluteus (iv), triceps (v), diaphragm (vi), heart (vii), and liver (viii) sections obtained from an mdx mouse 21 days post intracerebroventricular (ICV) injection with 9×10¹⁰ vg of INS1201-AAV9 and immunofluorescently stained for dystrophin.

FIG. 5 shows gastrocnemius (i), tibialis anterior (ii), quadriceps (iii), gluteus (iv), triceps (v), diaphragm (vi), heart (vii), and liver (viii) sections obtained from an mdx mouse 21 days post intracerebroventricular (ICV) injection with 9×10¹⁰ vg of INS1212-AAV9 and immunofluorescently stained for dystrophin.

FIG. 6A shows gastrocnemius muscle sections obtained from mdx mice 80 days post intracerebroventricular (ICV) injection with 9×10¹⁰ vg (ii) or 2.7×10¹¹ vg (iii) of INS1201-AAV9 and stained with hematoxylin and eosin (H&E). Gastrocnemius sections obtained from a wildtype C57/Bl mouse (i) and non-injected mdx mouse (iv) and H&E stained are shown for comparison.

FIG. 6B shows gastrocnemius muscle sections obtained from mdx mice 80 days post intracerebroventricular (ICV) injection with 9×10¹⁰ vg (ii) or 2.7×10¹¹ vg (iii) of INS1201-AAV9 and stained for dystrophin. Gastrocnemius sections obtained from a wildtype C57/Bl mouse (i) and non-injected mdx mouse (iv) and stained for dystrophin are shown for comparison.

FIG. 7A shows a gastrocnemius muscle section obtained from an mdx mouse 80 days post intracerebroventricular (ICV) injection with 9×10¹⁰ vg (ii) of INS1212-AAV9 and stained with hematoxylin and eosin (H&E). Gastrocnemius sections obtained from a wildtype C57/Bl mouse (i) and non-injected mdx mouse (iii) and H&E stained are shown for comparison.

FIG. 7B shows a gastrocnemius muscle section obtained from an mdx mouse 80 days post intracerebroventricular (ICV) injection with 9×10¹⁰ vg (ii) of INS1212-AAV9 and stained for dystrophin. Gastrocnemius sections obtained from a wildtype C57/Bl mouse (i) and non-injected mdx mouse (iii) and stained for dystrophin are shown for comparison.

FIG. 8A shows a bar graph of mean fiber diameter (μm) in gastrocnemius muscle cells in an mdx mouse 80 days post intracerebroventricular (ICV) injection with 9×10¹⁰ vg or 2.7×10¹¹ vg of INS1201-AAV9. Mean fiber diameters (μm) in gastrocnemius muscle cells in wildtype C57/Bl and non-injected mdx mice are shown for comparison.

FIG. 8B shows a line graph of relative frequencies (%) of cell diameters (μm) in gastrocnemius muscle cells in an mdx mouse 80 days post intracerebroventricular (ICV) injection with 9×10¹⁰ vg or 2.7×10¹¹ vg of INS1201-AAV9. Relative frequencies of cell diameters in gastrocnemius muscle cells in wildtype C57/Bl and non-injected mdx mice are shown for comparison.

FIG. 8C shows a bar graph of mean fiber diameter (μm) in triceps muscle cells in an mdx mouse 80 days post intracerebroventricular (ICV) injection with 9×10¹⁰ vg or 2.7×10¹¹ vg of INS1201-AAV9. Mean fiber diameters (μm) in triceps muscle cells in wildtype C57/Bl and non-injected mdx mice are shown for comparison.

FIG. 8D shows a line graph of relative frequencies (%) of cell diameters (μm) in triceps muscle cells in an mdx mouse 80 days post intracerebroventricular (ICV) injection with 9×10¹⁰ vg or 2.7×10¹¹ vg of INS1201-AAV9. Relative frequencies of cell diameters in triceps muscle cells in wildtype C57/Bl and non-injected mdx mice are shown for comparison.

FIG. 8E shows a bar graph of mean fiber diameter (μm) in tibialis anterior muscle cells in an mdx mouse 80 days post intracerebroventricular (ICV) injection with 9×10¹⁰ vg or 2.7×10¹¹ vg of INS1201-AAV9. Mean fiber diameters (μm) in tibialis anterior muscle cells in wildtype C57/Bl and non-injected mdx mice are shown for comparison.

FIG. 8F shows a line graph of relative frequencies (%) of cell diameters (μm) in tibialis anterior muscle cells in an mdx mouse 80 days post intracerebroventricular (ICV) injection with 9×10¹⁰ vg or 2.7×10¹¹ vg of INS1201-AAV9. Relative frequencies of cell diameters in tibialis anterior muscle cells in wildtype C57/Bl and non-injected mdx mice are shown for comparison.

FIG. 8G shows a bar graph of mean fiber diameter (μm) in diaphragm muscle cells in an mdx mouse 80 days post intracerebroventricular (ICV) injection with 9×10¹⁰ vg or 2.7×10¹¹ vg of INS1201-AAV9. Mean fiber diameters (μm) in diaphragm muscle cells in wildtype C57/Bl and non-injected mdx mice are shown for comparison.

FIG. 8H shows a line graph of relative frequencies (%) of cell diameters (in μm) in diaphragm muscle cells in an mdx mouse 80 days post intracerebroventricular (ICV) injection with 9×10¹⁰ vg or 2.7×10¹¹ vg of INS1201-AAV9. Relative frequencies of cell diameters in diaphragm muscle cells in wildtype C57/Bl and non-injected mdx mice are shown for comparison.

FIG. 9A shows a bar graph of mean fiber diameter (μm) in gastrocnemius muscle cells in an mdx mouse 80 days post intracerebroventricular (ICV) injection with 9×10¹⁰ vg of INS1212-AAV9. Mean fiber diameters (μm) in diaphragm muscle cells in wildtype C57/Bl and non-injected mdx mice are shown for comparison.

FIG. 9B shows a line graph of relative frequencies (%) of cell diameters (μm) in gastrocnemius muscle cells in an mdx mouse 80 days post intracerebroventricular (ICV) injection with 9×10¹⁰ vg of INS1212-AAV9. Relative frequencies of cell diameters in diaphragm muscle cells in wildtype C57/Bl and non-injected mdx mice are shown for comparison.

FIG. 10A is a line graph of percent contractile force in EDL muscle resulting from eccentric contractions (EC) in a wildtype C57/Bl mouse, an mdx mouse receiving intracerebroventricular (ICV) injection at postnatal day 1 (μl) with vehicle, an mdx mouse receiving ICV injection at postnatal day 1 μl of 2.7×10¹¹ vg of INS1201-AAV9, and an mdx mouse receiving ICV injection at μl of 9×10¹⁰ vg of INS1201-AAV9.

FIG. 10B is a bar graph of percent post-eccentric contraction (EC) stress in EDL muscle relative to pre-EC stress in (i) a wildtype C57/Bl mouse, and mdx mice receiving intracerebroventricular (ICV) injection at postnatal day 1 (μl) of (ii) 9×10⁹ vg of INS1201-AAV9; (iii) 9×10¹⁰ vg of INS1201-AAV9; (iv) 2.7×10¹¹ vg of INS1201-AAV9 or (v) vehicle control.

FIG. 10C is a graph showing the percent of contractile force in EDL muscle (% eccentric contraction 1 (EC1)) as a function of the eccentric contraction (EC) number in (i) a wildtype C57/Bl mouse, and mdx mice receiving intracerebroventricular (ICV) injection at postnatal day 28 (p28) of either (ii) 9×10¹⁰ vg of INS1201-AAV9; (iii) 2.7×10¹¹ vg of INS1201-AAV9; (iv) 5.4×10¹¹ vg of INS1201-AAV9; (v) 1.2×10¹² vg of INS1201-AAV9; or (vi) vehicle control.

FIG. 10D is a bar graph showing the percent of force in EDL muscle ([post EC5/post EC1) in (i) a wildtype C57/Bl mouse, and mdx mice receiving intracerebroventricular (ICV) injection at postnatal day 28 (p28) of either (ii) 9×10¹⁰ vg of INS1201-AAV9; (iii) 2.7×10¹¹ vg of INS1201-AAV9; (iv) 5.4×10¹¹ vg of INS1201-AAV9; (v) 1.2×10¹² vg of INS1201-AAV9; or (vi) vehicle control.

FIG. 10E is a graph of maximum tension (kPa) in EDL muscle resulting from eccentric contractions (EC), in (i) a wildtype C57/Bl mouse, and mdx mice receiving intracerebroventricular (ICV) injection at postnatal day 28 (p28) of either (ii) 9×10⁹ vg of INS1201-AAV9; (iii) 9×10¹⁰ vg of INS1201-AAV9; (iv) 2.7×10¹¹ vg of INS1201-AAV9; (v) 5.4×10¹¹ vg of INS1201-AAV9; (vi) 1.2×10¹² vg of INS1201-AAV9; or (vii) vehicle control.

FIG. 10F is a graph of peak stress (kPa) resulting from eccentric contractions (EC) at various frequencies (Hz), in (i) a wildtype C57/Bl mouse, and mdx mice receiving intracerebroventricular (ICV) injection at postnatal day 28 (p28) of either (ii) 9×10⁹ vg of INS1201-AAV9; (iii) 9×10¹⁰ vg of INS1201-AAV9; (iv) 2.7×10¹¹ vg of INS1201-AAV9; (v) 5.4×10¹¹ vg of INS1201-AAV9; (vi) 1.2×10¹² vg of INS1201-AAV9; or (vii) vehicle control.

FIG. 11A shows gastrocnemius muscle sections obtained from non-injected cynomolgus macaques (i), cynomolgus macaques 21 days post intravenous (IV) injections with 5×10¹³ vg (ii) or 1×10¹⁴ vg (iii) of AAV9 CBA-GFP; or cynomolgus macaques 21 days post intrathecal (IT) injections with 2.5×10¹³ vg (iv), 5×10¹³ vg (v), or 1×10¹⁴ vg (vi) of AAV9 CBA-GFP and immunostained with NovaRed™ for detection of GFP expression.

FIG. 11B shows quadriceps muscle sections obtained from non-injected cynomolgus macaques (i), cynomolgus macaques 21 days post intravenous (IV) injections with 5×10¹³ vg (ii) or 1×10¹⁴ vg (iii) of AAV9 CBA-GFP; or cynomolgus macaques 21 days post intrathecal (IT) injections with 2.5×10¹³ vg (iv), 5×10¹³ vg (v), or 1×10¹⁴ vg (vi) of AAV9 CBA-GFP and immunostained with NovaRed™ for detection of GFP expression.

FIG. 11C shows deltoid muscle sections obtained from non-injected cynomolgus macaques (i); cynomolgus macaques 21 days post intravenous (IV) injections with 5×10¹³ vg (ii) or 1×10¹⁴ vg (iii) of AAV9 CBA-GFP; or cynomolgus macaques 21 days post intrathecal (IT) injections with 2.5×10¹³ vg (iv), 5×10¹³ vg (v), or 1×10¹⁴ vg (vi) of AAV9 CBA-GFP and immunostained with NovaRed™ for detection of GFP expression.

FIG. 11D shows triceps muscle sections obtained from non-injected cynomolgus macaques (i); cynomolgus macaques 21 days post intravenous (IV) injections with 5×10¹³ vg (ii) or 1×10¹⁴ vg (iii) of AAV9 CBA-GFP; or cynomolgus macaques 21 days post intrathecal (IT) injections with 2.5×10¹³ vg (iv), 5×10¹³ vg (v), or 1×10¹⁴ vg (vi) of AAV9 CBA-GFP and immunostained with NovaRed™ for detection of GFP expression.

FIG. 11E shows biceps muscle sections obtained from non-injected cynomolgus macaques (i); cynomolgus macaques 21 days post intravenous (IV) injections with 5×10¹³ vg (ii) or 1×10¹⁴ vg (iii) of AAV9 CBA-GFP; or cynomolgus macaques 21 days post intrathecal (IT) injections with 2.5×10¹³ vg (iv), 5×10¹³ vg (v), or 1×10¹⁴ vg (vi) of AAV9 CBA-GFP and immunostained with NovaRed™ for detection of GFP expression.

FIG. 11F shows tibialis anterior muscle sections obtained from cynomolgus macaques 21 days post intravenous (IV) injections with 5×10¹³ vg (i) or 1×10¹⁴ vg (ii) of AAV9 CBA-GFP; or cynomolgus macaques 21 days post intrathecal (IT) injections with 2.5×10¹³ vg (iii), 5×10¹³ vg (iv), or 1×10¹⁴ vg (v) of AAV9 CBA-GFP and immunostained with NovaRed™ for detection of GFP expression.

FIG. 11G shows diaphragm muscle sections obtained from cynomolgus macaques 21 days post intravenous (IV) injections with 5×10¹³ vg (i) or 1×10¹⁴ vg (ii) of AAV9 CBA-GFP; or cynomolgus macaques 21 days post intrathecal (IT) injections with 2.5×10¹³ vg (iii), 5×10¹³ vg (iv), or 1×10¹⁴ vg (v) of AAV9 CBA-GFP and immunostained with NovaRed™ for detection of GFP expression.

FIG. 11H shows heart muscle sections obtained from cynomolgus macaques 21 days post intravenous (IV) injections with 5×10¹³ vg (i) or 1×10¹⁴ vg (ii) of AAV9 CBA-GFP; or cynomolgus macaques 21 days post intrathecal (IT) injections with 2.5×10¹³ vg (iii), 5×10¹³ vg (iv), or 1×10¹⁴ vg (v) of AAV9 CBA-GFP and immunostained with NovaRed™ for detection of GFP expression.

FIG. 11I shows liver sections obtained from cynomolgus macaques 21 days post intravenous (IV) injections with 5×10¹³ vg (i) or 1×10¹⁴ vg (ii) of AAV9 CBA-GFP; or cynomolgus macaques 21 days post intrathecal (IT) injections with 2.5×10¹³ vg (iii), 5×10¹³ vg (iv), or 1×10¹⁴ vg (v) of AAV9 CBA-GFP and immunostained with NovaRed™ for detection of GFP expression.

FIG. 12A shows a Ponceau stain (top panel) or Western Blot (bottom panel) of biceps (1), triceps (2), deltoid (3), quadriceps (4), gastrocnemius (5), tibialis anterior (6), diaphragm (7), and heart (8) muscle protein samples obtained from cynomolgus macaques 21 days post intrathecal (IT) injections with 2.5×10¹³ vg of AAV9 CBA-GFP and probed with anti-GFP antibody.

FIG. 12B shows a Ponceau stain (top panel) or Western Blot (bottom panel) of biceps (1), triceps (2), deltoid (3), quadriceps (4), gastrocnemius (5), tibialis anterior (6), diaphragm (7), and heart (8) muscle protein samples obtained from cynomolgus macaques 21 days post intrathecal (IT) injections with 5×10¹³ vg of AAV9 CBA-GFP and probed with anti-GFP antibody.

FIG. 12C shows a Ponceau stain (top panel) or Western Blot (bottom panel) of biceps (1), triceps (2), deltoid (3), quadriceps (4), gastrocnemius (5), tibialis anterior (6), diaphragm (7), and heart (8) muscle protein samples obtained from cynomolgus macaques 21 days post intrathecal (IT) injections with 1×10¹⁴ vg of AAV9 CBA-GFP and probed with anti-GFP antibody.

FIG. 12D shows a Ponceau stain (top panel) or Western Blot of biceps (1), and triceps (2) muscle protein samples obtained from non-injected cynomolgus macaques and probed with anti-GFP antibody.

FIG. 13 shows an agarose gel electrophoresis of biceps (1), triceps (2), deltoid (3), tibialis anterior (4), gastrocnemius (5), quadriceps vastus lateralis (6), diaphragm (7), and heart (8) muscle and liver (9) tissue samples obtained from cynomolgus macaques 21 days post intrathecal (IT) injections with 2.5×10¹³ vg of AAV9 CBA-GFP and subjected to RT-PCR in the presence (+) or absence (−) of reverse transcriptase. Biceps (10), triceps (11), deltoid (12), and quadriceps (13) muscle tissue samples obtained from non-injected cynomolgous macaques and subjected to RT-PCR in the presence (+) or absence (−) of reverse transcriptase are shown for comparison.

FIG. 14 shows various μDys protein domains encoded by μDys transgenes provided herein.

FIG. 15 is a graph showing the number of INS1201 DNA copies per diploid genome in mdx mice receiving intracerebroventricular (ICV) injection at postnatal day 28 (p28) of either (ii) 9×10⁹ vg of INS1201-AAV9; (iii) 9×10¹⁰ vg of INS1201-AAV9; (iv) 2.7×10¹¹ vg of INS1201-AAV9; (v) 5.4×10¹¹ vg of INS1201-AAV9; (vi) 1.2×10¹² vg of INS1201-AAV9; or (vii) vehicle control.

FIG. 16 is a graph showing the number of INS1201 RNA transcript copies normalized to the number of copies of RPP30 in mdx mice receiving intracerebroventricular (ICV) injection at postnatal day 28 (p28) of either (ii) 9×10⁹ vg of INS1201-AAV9; (iii) 9×10¹⁰ vg of INS1201-AAV9; (iv) 2.7×10¹¹ vg of INS1201-AAV9; (v) 5.4×10¹¹ vg of INS1201-AAV9; (vi) 1.2×10¹² vg of INS1201-AAV9; or (vii) vehicle control.

FIG. 17 is a graph of mean fiber diameter (μm) in EDL muscle cells at postnatal day 120 (p120) in (i) wildtype C57/Bl mice, and mdx mice receiving intracerebroventricular (ICV) injection at postnatal day 28 (p28) of either (ii) 9×10⁹ vg of INS1201-AAV9; (iii) 9×10¹⁰ vg of INS1201-AAV9; (iv) 2.7×10¹¹ vg of INS1201-AAV9; (v) 5.4×10¹¹ vg of INS1201-AAV9; (vi) 1.2×10¹² vg of INS1201-AAV9; or (vii) vehicle control.

FIG. 18 is a graph of mean fiber diameter (μm) in tibialis anterior muscle cells at postnatal day 120 (p120) in (i) wildtype C57/Bl mice, and mdx mice receiving intracerebroventricular (ICV) injection at postnatal day 28 (p28) of either (ii) 9×10⁹ vg of INS1201-AAV9; (iii) 9×10¹⁰ vg of INS1201-AAV9; (iv) 2.7×10¹¹ vg of INS1201-AAV9; (v) 5.4×10¹¹ vg of INS1201-AAV9; (vi) 1.2×10¹² vg of INS1201-AAV9; or (vii) vehicle control.

FIG. 19 shows diaphragm muscle sections stained with picrosirius red, post intracerebroventricular (ICV) injection with various doses of INS1201-AAV9. Diaphragm sections obtained from a wildtype C57/Bl mouse and mdx mouse administered vehicle are shown for comparison in the top section.

FIG. 20 is a graph showing the percent collagen in diaphragm muscle at postnatal day 120 (p120), in (i) wildtype C57/Bl mice, and mdx mice receiving intracerebroventricular (ICV) injection at postnatal day 28 (p28) of either (ii) vehicle control; (iii) 9×10⁹ vg of INS1201-AAV9; (iv) 9×10¹⁰ vg of INS1201-AAV9; (v) 2.7×10¹¹ vg of INS1201-AAV9; (v) 5.4×10¹¹ vg of INS1201-AAV9 or (vi) 1.2×10¹² vg of INS1201-AAV9.

FIG. 21, top, shows an extensor digitorum longus (EDL) muscle section obtained from an mdx mouse at postnatal day 120 (p120) subsequent to intracerebroventricular (ICV) injection at p28 with 5.4×10¹¹ vg of INS1201-AAV9 and stained with hematoxylin and eosin (H&E) (far left); laminin/dapi (second from left); dystrophin (second from right); and a merged image (far right). FIG. 21, bottom, shows a EDL muscle section obtained from an mdx mouse at postnatal day 120 (p120) subsequent to intracerebroventricular (ICV) injection at p28 with 5vehicle control, and stained with hematoxylin and eosin (H&E) (far left); laminin/dapi (second from left); dystrophin (second from right); and a merged image (far right).

### DETAILED DESCRIPTION OF THE INVENTION

The present invention relates in part to adeno-associated viral (AAV) particles and methods for preferentially delivering the same to cardiac and/or skeletal muscle to a subject in need of treatment of a monogenic muscle disease, for example, a dystrophinopathy such as Duchenne muscular dystrophy (DMD), Becker muscular dystrophy, or DMD-associated dilated cardiomyopathy (DCM). Without wishing to be bound by theory, the particles and methods for delivering the same to subjects in need thereof, for example to treat a monogenic muscle disease such as a dystrophinopathy, provide a superior benefit to known AAV particles and treatment methods at least because the present invention: (i) allows for significantly lower dosages than IV delivery, in order to achieve substantially the same or a better therapeutic benefit, thereby reducing viral load and toxicity and other side effects; and/or (ii) allows for preferential transgene targeting and expression in cardiac and/or skeletal muscle tissue compared to liver tissue, thereby targeting the transgene to cells of interest to provide a greater therapeutic benefit compared to AAV vectors delivered intravenously; (iii) can benefit greater patient populations compared to IV formulations, because of the lower dosages needed, corresponding to a decreased manufacturing burden.

Aspects of the invention relate to AAV particles, methods for producing the same, and methods for delivering the AAV particles to a subject in need of treatment. The AAV particle for example, an AAV9 particle, comprises a capsid comprising one or more AAV9 capsid proteins and a vector genome encapsidated by the AAV9 capsid. The vector genome comprises a transgene and regulatory elements that promote gene expression of the transgene when delivered into muscle cells, for example skeletal and/or cardiac muscle cells. In one embodiment, the transgene is a micro-dystrophin (μDys) transgene.

Methods described herein comprise intrathecally administering to a subject in need of treatment of a dystrophinopathy, in a single dose, a composition comprising an effective amount of an AAV particle as described herein. The dystrophinopathy in one embodiment, is DMD, Becker muscular dystrophy, or DCM. In a preferred embodiment, the dystrophinopathy is DMD. In embodiments described herein, intrathecal delivery of AAV particles of the present invention to muscle cells can result in robust expression of μDys and can significantly improve muscle health and function. The present invention also provides methods and cells for producing the AAV particles described herein. In one embodiment of the methods described herein, subsequent to intrathecal administration of the AAV particle, the transgene is expressed at higher levels in the skeletal and/or cardiac muscle of the subject, compared to the transgene expression in liver tissue of the subject.

To facilitate an understanding of the present invention, a number of terms and phrases are defined below.

The terms “a” and “an” as used herein mean “one or more” and include the plural unless the context is inappropriate.

The term “nucleic acid,” “nucleotide,” or “oligonucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” can refer to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, guide RNA (gRNA), short-interfering RNA (siRNA), or micro RNA (miRNA).

As used herein, the term “transgene” refers to an exogenous gene present in a vector genome, artificially introduced into the genome of a cell, or an endogenous gene artificially introduced into a non-natural locus in the genome of a cell. A transgene can refer to a segment of DNA involved in producing or encoding a polypeptide chain. Transgenes may include regions preceding and following the coding region (leader and trailer) as well as intervening sequences (introns) between individual coding segments (exons). The transgene according to embodiments described herein, is μDys. The μDys transgene, in one embodiment, encodes a μDys polypeptide comprising an N-terminal region, from about two to three hinge regions, from about four to six spectrin repeats, and a cysteine rich domain.

“Vector genome” as used herein, is a nucleic acid genome comprising one or more heterologous nucleic acid sequences. The one or more heterologous nucleic acid sequences comprises a transgene. In some embodiments of the invention, the vector genome comprises at least one ITR sequence (e.g., AAV ITR sequence), optionally two ITRs (e.g., two AAV ITRs), which typically will be at the 5′ and 3′ ends of the vector genome and flank the one or more heterologous nucleic acids. The ITRs can be the same or different from each other.

As used herein, the term “endogenous” with reference to a nucleic acid, for example, a gene, or a protein in a cell, is a nucleic acid or protein that occurs in that particular cell as it is found in nature, for example, at its natural genomic location or locus. Moreover, a cell “endogenously expressing” a nucleic acid or protein expresses that nucleic acid or protein as it is found in nature.

A “promoter” is defined as one or more nucleic acid control sequence(s) that direct transcription of a nucleic acid, e.g., a transgene, and can be present within a vector genome. As used herein, a promoter includes nucleic acid sequences near the start site of transcription. A promoter also optionally includes distal enhancer or repressor elements, which can be located as much as several thousand base pairs from the start site of transcription.

A “regulatory element” as used herein refers to a nucleic acid sequence capable of regulating transcription of a gene (e.g., a transgene), and/or regulate the stability or translation of a transcribed mRNA product, and can be present within a vector genome. In some embodiments, regulatory elements can regulate tissue-specific transcription of a gene. Regulatory elements can comprise at least one transcription factor binding site, for example, a transcription factor binding site for a muscle-specific transcription factor. Regulatory elements as used herein increase or enhance promoter-driven gene expression when compared to the transcription of the gene from the promoter alone in the absence of the regulatory element. Regulatory elements as used herein may occur at any distance (i.e., proximal or distal) to the transgene they regulate. Regulatory elements as used herein may comprise part of a larger sequence involved in transcriptional control, e.g., part of a promoter sequence. However, regulatory elements alone are typically not sufficient to initiate transcription on its own and require the presence of a promoter.

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

“Polypeptide,” “peptide,” and “protein” are used interchangeably herein to refer to a polymer of amino acid residues. As used herein, the terms encompass amino acid chains of any length, including full-length proteins, and functional fragments thereof, wherein the amino acid residues are linked by covalent peptide bonds.

As used herein, the term “complementary” or “complementarity” refers to specific base pairing between nucleotides or nucleic acids. Complementary nucleotides are, generally, A and T (or A and U), and G and C. The guide RNAs (gRNAs) described herein can comprise sequences, for example, DNA targeting sequences that are perfectly complementary or substantially complementary (e.g., having a small fraction of mismatched bases) to a genomic sequence.

As used herein, the terms “introducing” or “delivering” in the context of nucleic acids, for example, AAV vectors, refers to the translocation of the nucleic acid from outside a cell to inside the cell, for example, a muscle cell. In some cases, introducing refers to translocation of the nucleic acid from outside the cell to inside the nucleus of the cell. Various methods of such translocation are contemplated, including but not limited to, electroporation, contact with nanowires or nanotubes, receptor mediated internalization, translocation via cell penetrating peptides, liposome-mediated translocation, and the like.

As used herein, the terms “packaged” or “encapsidated” refers to the inclusion of a vector genome in a capsid comprising viral capsid proteins to form an AAV particle.

The term “substantial identity” or “substantially identical,” as used in the context of polynucleotide or polypeptide sequences, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10⁻²⁰.

As used herein, the terms “subject” and “patient” refer to an organism to be treated by the methods and compositions described herein. Such organisms include, but are not limited to, mammals such as humans, simians, murines, equines, bovines, porcines, canines, felines, and the like. In some embodiments, the subject or patient is human. The subject in the methods of treatment provided herein, in one embodiment, is a male subject.

In embodiments where the subject is a male human, the male human subject is from about 4 years old to about 7 years old, a newborn, about 1 year to about 7 years old, about 2 years to about 7 years old, about 2 years to about 6 years old, about 2 years to about 5 years old, about 2 years to about 4 years old, about 3 years to about 7 years old, about 3 years to about 6 years old, about 1 month to about 6 years old, about 1 month to about 5 years old, about 1 month to about 4 years old, about 1 month to about 3 years old, about 1 month to about 2 years old, or about 1 month to about 12 months old.

In one embodiment, the subject is a male human patient from about 4 years old to about 7 years old. In one embodiment, the subject is a male human patient from about 3 years old to about 7 years old. In one embodiment, the subject is a male human patient from about 2 years old to about 7 years old.

As used herein, the term “efficient delivery” or “efficiently delivering” refers to administration of an AAV particle comprising an AAV capsid encapsidating a vector genome encoding a transgene that results in expression of the transgene in a desired cell or tissue.

As used herein, the term “effective amount” or “effective dose”, refers to the amount of a substance (e.g., an AAV particle of the present invention) sufficient to effect beneficial or desired results (e.g., expression of a protein, or a desired prophylactic or therapeutic effect). An effective amount can be administered in one or more administration(s), application(s) or dosage(s) and is not intended to be limited to a particular formulation or administration route. Where a dose is provided in “vector genomes”, an “effective dose” may be referred to herein as an “effective vector genome dose”.

As used herein, the term “treating” includes any effect, e.g., lessening, reducing, modulating, ameliorating or eliminating, that results in the improvement of the condition, disease, disorder, and the like, or ameliorating a symptom thereof.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

### Adeno-Associated Virus (AAV) Particles

As described herein, one aspect of the present invention relates to an AAV particle comprising one or more AAV capsid proteins and a vector genome encapsidated by the one or more capsid proteins; and intrathecal compositions comprising the same. The genome comprises from 5′ to 3′, a 5′ inverted terminal repeat (ITR), a promoter, a transgene, an SV40 poly (A) tail; and a 3′ ITR. When intrathecally administered to subjects in need of treatment, in one embodiment, the transgene is expressed at higher levels in the skeletal and/or cardiac muscle of the subject, compared to the transgene expression in liver tissue of the subject. In another embodiment, the ratio of [(skeletal and/or cardiac muscle transgene expression)]/(liver transgene expression)] provided by an AAV particle described herein, is greater than the same ratio when the identical dose of the same AAV particle is administered intravenously. In one preferred embodiment, the AAV particle is an AAV9 particle comprising one or more AAV9 capsid proteins.

As used herein, an “adeno-associated virus (AAV) particle”, refers to an AAV virion comprising an AAV capsid and a vector genome encapsidated by the AAV capsid. The vector genome typically comprises a promoter and one or more transgenes, that are flanked by AAV ITR sequences. The AAV capsid comprises one or more AAV capsid proteins. The AAV capsid proteins can be from the same or different AAV serotypes and can be wild-type or engineered. Vector genomes described herein can be replicated, and packaged into viral vectors (particles) when introduced into a host cell also comprising one or more plasmids encoding rep and cap gene products. In one embodiment, a helper plasmid is also transfected into the host cell to aid in vector production by the host cell. In one embodiment, the AAV vector used herein is an AAV9 vector, for example, as described in U.S. Pat. No. 7,906,111, the disclosure of which is incorporated by reference herein in its entirety for all purposes. In one embodiment, the AAV capsid is an AAV9 capsid.

The terms “empty capsid,” “empty vial particle,” and “empty AAV” refer to an AAV capsid shell lacking a vector genome packaged within.

Encapsidated vector genomes described herein can include one or more regulatory elements, for example one or more regulatory elements upstream of the transgene. The encapsidated genome, in one embodiment, comprises from 5′ to 3′, a 5′ ITR, a promoter, a transgene, an SV40 poly (A) tail; and a 3′ ITR. In another embodiment, the encapsidated genome comprises from 5′ to 3′, a 5′ ITR, an enhancer, a promoter, a transgene, an SV40 poly (A) tail; and a 3′ ITR. The encapsidated genome, in even another embodiment, comprises from 5′ to 3′, a 5′ ITR, a promoter, a SV40 intron, a transgene, an SV40 poly (A) tail; and a 3′ ITR. In yet even another embodiment, the encapsidated genome comprises from 5′ to 3′, a 5′ ITR, an enhancer, a promoter, an SV40 intron, a transgene, an SV40 poly (A) tail; and a 3′ ITR.

### Inverted Terminal Repeats

Inverted terminal repeats (ITR) are palindromic 145 nucleotide sequences that flank a transgene. The 5′ and 3′ ITRs of an AAV vector genome are necessary for both the integration of the transgene into the host cell genome (e.g., chromosome 19 in humans) and for encapsidation of the transgene into the AAV particle.

In some embodiments, AAV vector genomes of the present invention comprise ITR sequences from any one AAV serotype, for example, AAVrh.74, AAV1, AAV2, AAV3, AAV4, AAV5, AAV6, AAV8, AAV9, AAV10, AAV11, AAV12, or AAV13. In some embodiments, the AAV vector genomes disclosed herein comprise a 5′ AAV2 ITR and a 3′ AAV2 ITR sequence.

In some embodiments, AAV vector genomes described herein comprise a 5′ AAV2 ITR having at least 80%, 85%, 90%, 91%, 92%, 93%, 94%, 95%, 96%, 97%, 98%, 99%, or 100% sequence identity to SEQ ID NO:1 (see Table 1). In some embodiments, the 5′ AAV2 ITR comprises SEQ ID NO:1. In some embodiments, the 5′ AAV2 ITR consists of SEQ ID NO:1.

In some embodiments, AAV vector genomes described herein comprise a 3′ AAV2 ITR having at least 80%, 85%, 90%, 91%, 92%, 93%, 94%, 95%, 96%, 97%, 98%, 99%, or 100% sequence identity to SEQ ID NO:7 (see Table 1). In some embodiments, the 3′ AAV2 ITR comprises SEQ ID NO:7. In some embodiments, the 3′ AAV2 ITR consists of SEQ ID NO:7.

### Promoters

Promoters drive the expression of the transgene and are typically located upstream (or 5′) of the transgene whose expression they regulate.

In some embodiments, AAV vector genomes of the present invention comprise a mammalian promoter, for example, human, non-human primate (e.g., cynomolgous macaque), mouse, horse, cow, pig, cat, and dog promoters. In some embodiments, recombinant AAV vector genomes disclosed herein comprise strong, constitutively active promoters to drive high-level expression of the transgene. For example, in some embodiments, the promoter is a cytomegalovirus (CMV) promoter/enhancer, an elongation factor 1α (EF1α) promoter, a simian virus 40 (SV40) promoter, a chicken β-actin hybrid promoter, or a CAG promoter.

The promoter, in one embodiment, is an MHCK7 or chicken β-actin hybrid promoter.

In certain embodiments, an AAV vector genome of the present invention comprise a muscle-specific promoter that is operably linked to the transgene to drive high-level and tissue-specific expression in muscle cells. For example, muscle specific promoters of the present invention include, but are not limited to, promoters selected from: desmin (DES, also known as CSM1 or CSM2) promoter, the alpha 2 actinin (ACTN2, also known as CMD1AA) promoter, the filamin-C (FLNC, also known as actin-binding-like protein (ABLP), filamin-2 (FLN2), ABP-280, ABP280A, ABPA, ABPL, MFM5 or MPD4) promoter, the sarcoplasmic/endoplasmic reticulum calcium ATPase 1 (ATP2A1, also known as ATP2A or SERCA1) promoter, the troponin I type 1 (TNNI1, also known as SSTNI or 25TTNI) promoter, the myosin-1 (MYH1) promoter, the phosphorylatable, fast skeletal muscle myosin light chain (MYLPF) promoter, myosin 1 (MYH1, also known as MYHSA1, MYHa, MyC-2X/D or MyHC-2x) promoter, the alpha-3 chain tropomyosin (TPM3, also known as CFTD, NEM1, OK/Scl.5, TM-5, TM3, TM30, TM30 nm, TM5, TPMsk3, TRK, h TM5 or hscp30) promoter, the ankyrin repeat domain-containing protein 2 (ANKRD2, also known as ARPP) promoter, the myosin heavy-chain (MHC) promoter, the myosin light-chain (MLC) promoter, the muscle creatine kinase (MCK) promoter, synthetic muscle promoters as described in Li et al. (1999. Nat Biotechnol. 17:241-245), such as the SPc5-12 promoter, the muscle creatine kinase (MCK) promoter, the dMCK promoter, the tMCK promoter consisting of respectively, a double or triple tandem of the MCK enhancer to the MCK basal promoter as described in Wang et al. (2008. Gene Ther. 15:1489-1499) and hybrid promoters such as the hybrid alpha-myosin heavy chain enhancer/MCK enhancer (MHCK7; 770 bp); the MCK-C5-12 promoter as described in Wang et al. (2008. Gene Ther. 15:1489-1499) and the cardiac and skeletal muscle-specific myosin chaperone Unc45b (195 bp) promoter as described in Rudeck S et al. (2016, Genesis. 54(8): 431-8). Non-limiting examples of heart-specific promoters include the calsequestrin 2 (also known as PDIB2, FLJ26321, FLJ93514 or CASQ2 (GeneID 845 for the human gene)) promoter, the ankyrin repeat domain 1 (also known as cardiac ankyrin repeat protein) promoter, the cytokine inducible nuclear protein promoter; the liver ankyrin repeat domain 1 (ANKRD1; GeneID 27063 for the human gene) promoter; the myosin, light chain 2, regulatory, cardiac, slow (MYL2; GeneID 4633 for the human gene) promoter; the myosin, light chain 3, alkali; ventricular, skeletal 10 slow (MYL3; GeneID 4634 for the human gene) promoter; the bromodomain containing 7 (also known as BP75, CELTIX1, NAG4(BRD7; GeneID 29117 for the human gene)) promoter; the alpha myosin heavy chain (αMHC) promoter; the cardiac troponin C promoter and the promoter of the cardiac sodium-calcium exchanger (NCX1), which confers cardiac specificity.

In some embodiments, AAV vector genomes described herein comprise a MHCK7 promoter. For example, in some embodiments the MHCK7 promoter has at least 80%, 85%, 90%, 91%, 92%, 93%, 94%, 95%, 96%, 97%, 98%, 99%, or 100% sequence identity to SEQ ID NO:2 (see Table 1). In some embodiments, the MHCK7 promoter comprises SEQ ID NO:2. In some embodiments, the MHCK7 promoter consists of SEQ ID NO:2.

### SV40 Intron

In some embodiments, AAV vector genomes of the present invention comprise an SV40 intron. The SV40 intron is a commonly used regulatory element in gene therapy vectors and enhances translation and stability of the expressed RNA transcript.

In certain embodiments, the SV40 intron is downstream (i.e., 3′) of the promoter and upstream (i.e., 5′) of the transgene. In other embodiments, the SV40 intron can be downstream (i.e., 3′) of the transgene.

In some embodiments, the SV40 intron has at least 80%, 85%, 90%, 91%, 92%, 93%, 94%, 95%, 96%, 97%, 98%, 99%, or 100% sequence identity to SEQ ID NO:4 (see Table 1). In some embodiments, the SV40 intron comprises SEQ ID NO:4. In some embodiments, the SV40 intron consists of SEQ ID NO:4.

### SV40-Poly(A) Tail

In some embodiments, AAV vector genomes of the present invention comprise a nucleic acid sequence encoding an SV40 poly(A) tail. The SV40-poly(A) tail sequence is a commonly used nucleic acid element in gene therapy vectors that assists in RNA export from the nucleus, translation of RNA, and RNA stability.

In some embodiments, AAV vector genomes of the present invention comprise a sequence encoding an SV40 poly(A) tail having at least 80%, 85%, 90%, 91%, 92%, 93%, 94%, 95%, 96%, 97%, 98%, 99%, or 100% sequence identity to SEQ ID NO:6 (see Table 1). In some embodiments, the sequence encoding the SV40 poly(A) tail comprises SEQ ID NO:6. In some embodiments, the sequence encoding the SV40 poly(A) tail consists of SEQ ID NO:6.

### Enhancers

In some embodiments, AAV vector genomes of the present invention comprise one or more enhancer sequence(s). An enhancer sequence, in one embodiment, can increase the level of transcription of the transgene, for example, by serving as a binding site for transcription factors and co-regulators that assist in DNA looping and recruitment of the transcriptional machinery to promoters.

In some embodiments, the enhancer is downstream (i.e., 3′) of the 5′ ITR and upstream (i.e., 5′) of the promoter. In some embodiments, the enhancer is downstream (i.e., 3′) of the promoter and upstream (i.e., 5′) of the transgene. In some embodiments, the enhancer is downstream (i.e., 3′) of the transgene and upstream (i.e., 5′) of the 3′ UTR.

In some embodiments, recombinant AAV vector genomes of the present invention comprise an enhancer that significantly promotes the transcription of a transgene in muscle cells, e.g., skeletal and/or cardiac muscle cells.

In some embodiments, recombinant AAV vectors described herein comprise a skeletal-cis-regulatory module 4 (SK-CRM4) enhancer. For example, in some embodiments the SK-CRM4 enhancer has at least 80%, 85%, 90%, 91%, 92%, 93%, 94%, 95%, 96%, 97%, 98%, 99%, or 100% sequence identity to SEQ ID NO:8 (see Table 1). In some embodiments, the SK-CRM4 enhancer comprises SEQ ID NO:8 In some embodiments, the SK-CRM4 enhancer consists of SEQ ID NO:8.

In yet another embodiment, an AAV vector genome of the present invention comprises a cytomegalovirus (CMV) enhancer nucleic acid sequence. In a further embodiment, the CMV enhancer is upstream of a promoter sequence. For example, in one embodiment, the CMV enhancer has the nucleic acid sequence of SEQ ID NO:9. In some embodiments, the CMV enhancer has at least 80%, 85%, 90%, 91%, 92%, 93%, 94%, 95%, 96%, 97%, 98%, 99%, or 100% sequence identity to SEQ ID NO:9 (see Table 1). In some embodiments, the CMV enhancer comprises SEQ ID NO:9. In some embodiments, the CMV enhancer consists of SEQ ID NO:9.

### Transgene

Transgenes for use with the present invention are nucleic acid sequences encoding a polypeptide, or functional fragment thereof, to be expressed in a cell (e.g., a muscle cell) into which the transgene is delivered.

In some embodiments, the transgene may be incorporated into the genome of the host cell to which it is delivered or may be expressed episomally.

In some embodiments, a transgene can encode a polypeptide, or functional fragment thereof, that is not endogenously expressed by the cell in which the transgene is delivered. In some embodiments, the transgene encodes a mutant form of a polypeptide, or functional fragment thereof, that is endogenously expressed by the cell in which the transgene is delivered. In some embodiments, the transgene encodes a protein, or functional fragment thereof, that is endogenously expressed by the cell in which the transgene is delivered but is expressed at low levels, and wherein expression of the transgene results in higher expression levels of the protein, or functional fragment thereof. In some embodiments, the cell in which the transgene is delivered harbors one or more mutation(s) that results in lowered levels of expression of an endogenous protein and/or a functionally defective protein, and wherein expression of the transgene results in restored expression of the endogenous protein and/or functional supplementation of the defective protein. In some embodiments, the transgene is silent when introduced into the cell in which the transgene is delivered and expression may be induced.

In some embodiments, the transgene may be heterologous (i.e., from a different species) or homologous (i.e., from the same species) to the promoter and/or other regulatory elements present in the recombinant AAV vectors described herein. In some embodiments, the transgene may be heterologous or homologous to the cell into which the transgene is delivered.

In some embodiments, the transgene may be a full length cDNA or genomic DNA sequence, or a fragment or mutant thereof having functional activity. In some embodiments, the transgene may be a minigene, i.e., a gene sequence lacking part, most or all of its intronic sequences or may contain all of its intron sequences. In some embodiments, the transgene may be a hybrid nucleic acid sequence comprising homologous and/or heterologous cDNA and/or genomic DNA fragments. In some embodiments, the transgene may comprise one or more nucleotide substitutions, deletions, and/or insertions as compared to a wildtype sequence.

In some embodiments, transgenes of the present invention encode a therapeutic protein. In certain embodiments, the transgene may encode a structural protein.

In some embodiments, the transgene is a micro-dystrophin (μDys) transgene that encodes a μDys polypeptide. In a further embodiment, the μDys polypeptide encoded by the transgene comprises (i) an N-terminal region (NTD) comprising an actin binding site, (ii) a central rod domain comprising from two to four hinge regions and from four to six spectrin repeats. In a further embodiment, the μDys transgene comprises the nucleic acid sequence set forth in SEQ ID NO:5. In even a further embodiment, the μDys transgene consists of the nucleic acid sequence set forth in SEQ ID NO:5. In another embodiment, the sequence encoding a μDys protein has at least 80%, 85%, 90%, 91%, 92%, 93%, 94%, 95%, 96%, 97%, 98%, 99%, or 100% sequence identity to SEQ ID NO:5.

The μDys transgene in one embodiment, comprises the nucleic acid sequence set forth at SEQ ID NO:4 of U.S. Pat. No. 10,351,611, incorporated by reference herein in its entirety for all purposes. In another embodiment, the sequence encoding a μDys protein has at least 80%, 85%, 90%, 91%, 92%, 93%, 94%, 95%, 96%, 97%, 98%, 99%, or 100% sequence identity to SEQ ID NO:4 of U.S. Pat. No. 10,351,611.

In one embodiment, the μDys transgene encodes a μDys polypeptide comprising (i) an N-terminal region (NTD) comprising an actin binding site, (ii) a domain comprising three hinge regions and four spectrin repeats, and (iii) a cysteine rich domain. The μDys transgene, in a further embodiment, encodes a μDys polypeptide comprising an N-terminal region (NTD) comprising an actin binding site, (ii) a central rod domain comprising hinge regions 1, 2 and 4, and spectrin repeats 1, 2, 3 and 24, and a (iii) cysteine rich domain.

The μDys transgene, in one embodiment, encodes dystrophin spectrin repeats 16 and 17, which have been reported as a scaffold for sarcolemmal neuronal nitric oxide synthase (nNOS) targeting. In a further embodiment, the μDys transgene encodes dystrophin spectrin repeats 1 and 24. In another embodiment, the μDys transgene encodes dystrophin spectrin repeats 1, 16 and 17, 23 and 24. In yet another embodiment, the μDys transgene encodes dystrophin spectrin repeats 1, 2, 3 and 24. In yet even another embodiment, the μDys transgene encodes dystrophin spectrin repeats 1, 2, 22, 23 and 24.

The μDys transgene, in one embodiment, encodes dystrophin hinge regions 1 and 4. In another embodiment, the μDys transgene encodes dystrophin hinge regions 1, 3 and 4.

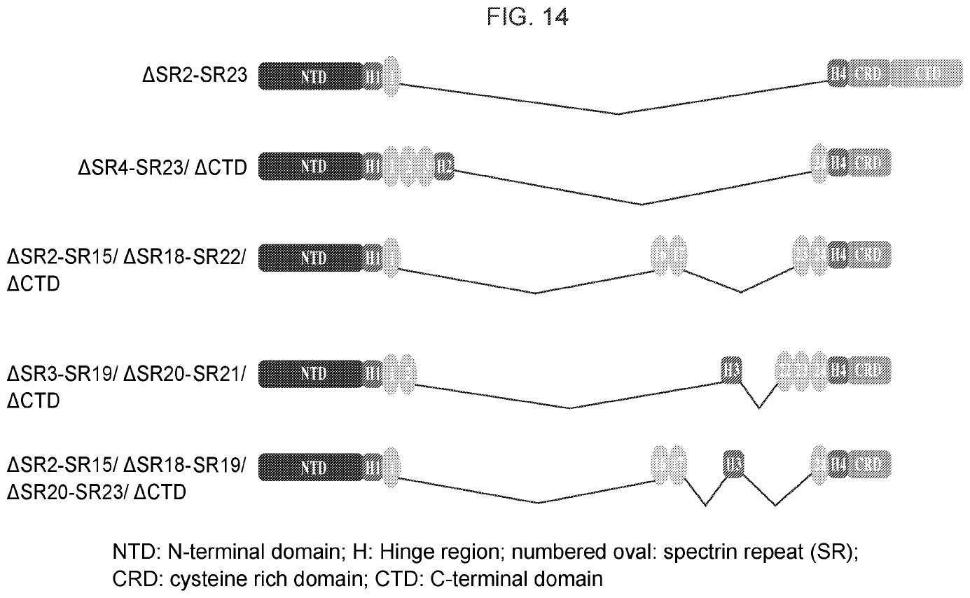

In one embodiment, the μDys transgene encodes a μDys protein comprising one of the combinations of μDys domains set forth in FIG. 14.

The AAV vector genomes described herein comprise a μDys transgene encoding a μDys protein comprising (i) an NTD comprising an actin binding site, (ii) a central rod domain comprising from two to four hinge regions and from four to six spectrin repeats, and (iii) a cysteine rich domain. For example, in one embodiment, the μDys transgene encodes dystrophin spectrin repeats 16 and 17. In a further embodiment, the μDys transgene encodes dystrophin spectrin repeats 1 and 24. In another embodiment, the μDys transgene encodes dystrophin spectrin repeats 1, 16 and 17, 23 and 24. In yet another embodiment, the μDys transgene encodes dystrophin spectrin repeats 1, 2, 3 and 24. In yet even another embodiment, the μDys transgene encodes dystrophin spectrin repeats 1, 2, 22, 23 and 24.

The μDys transgene in one embodiment, encodes dystrophin hinge regions 1 and 4. In another embodiment, the μDys transgene encodes dystrophin hinge regions 1, 3 and 4.

AAV particles described herein, in one embodiment, comprise an encapsidated transgene encoding a μDys protein. For example, in some embodiments the sequence encoding a μDys protein has at least 80%, 85%, 90%, 91%, 92%, 93%, 94%, 95%, 96%, 97%, 98%, 99%, or 100% sequence identity to SEQ ID NO:5 (see Table 1). In some embodiments, the sequence encoding a μDys protein comprises SEQ ID NO:5. In some embodiments, the sequence encoding a μDys protein consists of SEQ ID NO:5.

TABLE 1
Exemplary vector genome components for use with the present invention.

| Regulatory | |
|---|---|
| Element or | |
| Transgene | Sequence |
| 5′ AAV2 | CTGCGCGCTCGCTCGCTCACTGAGGCCGCCCGGGCAAAGCCCGG |
| ITR | GCGTCGGGCGACCTTTGGTCGCCCGGCCTCAGTGAGCGAGCGAG |
| | CGCGCAGAGAGGGAGTGGCCAACTCCATCACTAGGGGTTCCT |
| | [SEQ ID NO: 1] |
| MHCK7 | CCTTCAGATTAAAAATAACTGAGGTAAGGGCCTGGGTAGGGGAG |
| promoter | GTGGTGTGAGACGCTCCTGTCTCTCCTCTATCTGCCCATCGGCCC |
| | TTTGGGGAGGAGGAATGTGCCCAAGGACTAAAAAAAGGCCATG |
| | GAGCCAGAGGGGCGAGGGCAACAGACCTTTCATGGGCAAACCTT |
| | GGGGCCCTGCTGTCTAGCATGCCCCACTACGGGTCTAGGCTGCCC |
| | ATGTAAGGAGGCAAGGCCTGGGGACACCCGAGATGCCTGGTTAT |
| | AATTAACCCAGACATGTGGCTGCCCCCCCCCCCCCAACACCTGCT |
| | GCCTCTAAAAATAACCCTGTCCCTGGTGGATCCCCTGCATGCGAA |
| | GATCTTCGAACAAGGCTGTGGGGGACTGAGGGCAGGCTGTAACA |
| | GGCTTGGGGGCCAGGGCTTATACGTGCCTGGGACTCCCAAAGTA |
| | TTACTGTTCCATGTTCCCGGCGAAGGGCCAGCTGTCCCCCGCCAG |
| | CTAGACTCAGCACTTAGTTTAGGAACCAGTGAGCAAGTCAGCCC |
| | TTGGGGCAGCCCATACAAGGCCATGGGGCTGGGCAAGCTGCACG |
| | CCTGGGTCCGGGGTGGGCACGGTGCCCGGGCAACGAGCTGAAAG |
| | CTCATCTGCTCTCAGGGGCCCCTCCCTGGGGACAGCCCCTCCTGG |
| | CTAGTCACACCCTGTAGGCTCCTCTATATAACCCAGGGGCACAG |
| | GGGCTGCCCTCATTCTACCACCACCTCCACAGCACAGACAGACA |
| | CTCAGGAGCCAGCCAGCC |
| | [SEQ ID NO: 2] |
| Chicken β- | CCACGTTCTGCTTCACTCTCCCCATCTCCCCCCCCTCCCCACCCCC |
| actin-hybrid | AATTTTGTATTTATTTATTTTTTAATTATTTTGTGCAGCGATGGGG |
| promoter | GCGGGGGGGGGGGGGGGGCGCGCGCCAGGCGGGGGGGGCGGG |
| | GCGAGGGGCGGGGGGGGCGAGGCGGAGAGGTGCGGCGGCAGC |
| | CAATCAGAGCGGCGCGCTCCGAAAGTTTCCTTTTATGGCGAGGC |
| | GGCGGCGGCGGCGGCCCTATAAAAAGCGAAGCGCGCGGCGGGC |
| | GGGAG |
| | [SEQ ID NO: 3] |
| SV40 intron | GTAAGTTTAGTCTTTTTTGTCTTTTATTTCAGGTCCCGGATCCGGT |
| | GGTGGTGCAAATCAAAGAACTGCTCCTCAGTCGATGTTGCCTTTA |
| | CTTCTAG |
| | [SEQ ID NO: 4] |
| Micro- | ATGCTTTGGTGGGAAGAAGTAGAGGACTGTTATGAAAGAGAAGA |
| dystrophin | TGTTCAAAAGAAAACATTCACAAAATGGGTAAATGCACAATTTT |
| (μDys) | CTAAGTTTGGGAAGCAGCATATTGAGAACCTCTTCAGTGACCTA |
| transgene | CAGGATGGGAGGCGCCTCCTAGACCTCCTCGAAGGCCTGACAGG |
| | GCAAAAACTGCCAAAAGAAAAAGGATCCACAAGAGTTCATGCC |
| | CTGAACAATGTCAACAAGGCACTGCGGGTTTTGCAGAACAATAA |
| | TGTTGATTTAGTGAATATTGGAAGTACTGACATCGTAGATGGAA |
| | ATCATAAACTGACTCTTGGTTTGATTTGGAATATAATCCTCCACT |
| | GGCAGGTCAAAAATGTAATGAAAAATATCATGGCTGGATTGCAA |
| | CAAACCAACAGTGAAAAGATTCTCCTGAGCTGGGTCCGACAATC |
| | AACTCGTAATTATCCACAGGTTAATGTAATCAACTTCACCACCAG |
| | CTGGTCTGATGGCCTGGCTTTGAATGCTCTCATCCATAGTCATAG |
| | GCCAGACCTATTTGACTGGAATAGTGTGGTTTGCCAGCAGTCAG |
| | CCACACAACGACTGGAACATGCATTCAACATCGCCAGATATCAA |
| | TTAGGCATAGAGAAACTACTCGATCCTGAAGATGTTGATACCAC |
| | CTATCCAGATAAGAAGTCCATCTTAATGTACATCACATCACTCTT |
| | CCAAGTTTTGCCTCAACAAGTGAGCATTGAAGCCATCCAGGAAG |
| | TGGAAATGTTGCCAAGGCCACCTAAAGTGACTAAAGAAGAACAT |
| | TTTCAGTTACATCATCAAATGCACTATTCTCAACAGATCACGGTC |
| | AGTCTAGCACAGGGATATGAGAGAACTTCTTCCCCTAAGCCTCG |
| | ATTCAAGAGCTATGCCTACACACAGGCTGCTTATGTCACCACCTC |
| | TGACCCTACACGGAGCCCATTTCCTTCACAGCATTTGGAAGCTCC |
| | TGAAGACAAGTCATTTGGCAGTTCATTGATGGAGAGTGAAGTAA |
| | ACCTGGACCGTTATCAAACAGCTTTAGAAGAAGTATTATCGTGG |
| | CTTCTTTCTGCTGAGGACACATTGCAAGCACAAGGAGAGATTTCT |
| | AATGATGTGGAAGTGGTGAAAGACCAGTTTCATACTCATGAGGG |
| | GTACATGATGGATTTGACAGCCCATCAGGGCCGGGTTGGTAATA |
| | TTCTACAATTGGGAAGTAAGCTGATTGGAACAGGAAAATTATCA |
| | GAAGATGAAGAAACTGAAGTACAAGAGCAGATGAATCTCCTAA |
| | ATTCAAGATGGGAATGCCTCAGGGTAGCTAGCATGGAAAAACAA |
| | AGCAATTTACATAGAGTTTTAATGGATCTCCAGAATCAGAAACT |
| | GAAAGAGTTGAATGACTGGCTAACAAAAACAGAAGAAAGAACA |
| | AGGAAAATGGAGGAAGAGCCTCTTGGACCTGATCTTGAAGACCT |
| | AAAACGCCAAGTACAACAACATAAGGTGCTTCAAGAAGATCTAG |
| | AACAAGAACAAGTCAGGGTCAATTCTCTCACTCACATGGTGGTG |
| | GTAGTTGATGAATCTAGTGGAGATCACGCAACTGCTGCTTTGGA |
| | AGAACAACTTAAGGTATTGGGAGATCGATGGGCAAACATCTGTA |
| | GATGGACAGAAGACCGCTGGGTTCTTTTACAAGACATCCTTCTCA |
| | AATGGCAACGTCTTACTGAAGAACAGTGCCTTTTTAGTGCATGGC |
| | TTTCAGAAAAAGAAGATGCAGTGAACAAGATTCACACAACTGGC |
| | TTTAAAGATCAAAATGAAATGTTATCAAGTCTTCAAAAACTGGC |
| | CGTTTTAAAAGCGGATCTAGAAAAGAAAAAGCAATCCATGGGCA |
| | AACTGTATTCACTCAAACAAGATCTTCTTTCAACACTGAAGAATA |
| | AGTCAGTGACCCAGAAGACGGAAGCATGGCTGGATAACTTTGCC |
| | CGGTGTTGGGATAATTTAGTCCAAAAACTTGAAAAGAGTACAGC |
| | ACAGATTTCACAGGCTGTCACCACCACTCAGCCATCACTAACAC |
| | AGACAACTGTAATGGAAACAGTAACTACGGTGACCACAAGGGA |
| | ACAGATCCTGGTAAAGCATGCTCAAGAGGAACTTCCACCACCAC |
| | CTCCCCAAAAGAAGAGGCAGATTACTGTGGATCTTGAAAGACTC |
| | CAGGAACTTCAAGAGGCCACGGATGAGCTGGACCTCAAGCTGCG |
| | CCAAGCTGAGGTGATCAAGGGATCCTGGCAGCCCGTGGGCGATC |
| | TCCTCATTGACTCTCTCCAAGATCACCTCGAGAAAGTCAAGGCAC |
| | TTCGAGGAGAAATTGCGCCTCTGAAAGAGAACGTGAGCCACGTC |
| | AATGACCTTGCTCGCCAGCTTACCACTTTGGGCATTCAGCTCTCA |
| | CCGTATAACCTCAGCACTCTGGAAGACCTGAACACCAGATGGAA |
| | GCTTCTGCAGGTGGCCGTCGAGGACCGAGTCAGGCAGCTGCATG |
| | AAGCCCACAGGGACTTTGGTCCAGCATCTCAGCACTTTCTTTCCA |
| | CGTCTGTCCAGGGTCCCTGGGAGAGAGCCATCTCGCCAAACAAA |
| | GTGCCCTACTATATCAACCACGAGACTCAAACAACTTGCTGGGA |
| | CCATCCCAAAATGACAGAGCTCTACCAGTCTTTAGCTGACCTGA |
| | ATAATGTCAGATTCTCAGCTTATAGGACTGCCATGAAACTCCGA |
| | AGACTGCAGAAGGCCCTTTGCTTGGATCTCTTGAGCCTGTCAGCT |
| | GCATGTGATGCCTTGGACCAGCACAACCTCAAGCAAAATGACCA |
| | GCCCATGGATATCCTGCAGATTATTAATTGTTTGACCACTATTTA |
| | TGACCGCCTGGAGCAAGAGCACAACAATTTGGTCAACGTCCCTC |
| | TCTGCGTGGATATGTGTCTGAACTGGCTGCTGAATGTTTATGATA |
| | CGGGACGAACAGGGAGGATCCGTGTCCTGTCTTTTAAAACTGGC |
| | ATCATTTCCCTGTGTAAAGCACATTTGGAAGACAAGTACAGATA |
| | CCTTTTCAAGCAAGTGGCAAGTTCAACAGGATTTTGTGACCAGC |
| | GCAGGCTGGGCCTCCTTCTGCATGATTCTATCCAAATTCCAAGAC |
| | AGTTGGGTGAAGTTGCATCCTTTGGGGGCAGTAACATTGAGCCA |
| | AGTGTCCGGAGCTGCTTCCAATTTGCTAATAATAAGCCAGAGAT |
| | CGAAGCGGCCCTCTTCCTAGACTGGATGAGACTGGAACCCCAGT |
| | CCATGGTGTGGCTGCCCGTCCTGCACAGAGTGGCTGCTGCAGAA |
| | ACTGCCAAGCATCAGGCCAAATGTAACATCTGCAAAGAGTGTCC |
| | AATCATTGGATTCAGGTACAGGAGTCTAAAGCACTTTAATTATG |
| | ACATCTGCCAAAGCTGCTTTTTTTCTGGTCGAGTTGCAAAAGGCC |
| | ATAAAATGCACTATCCCATGGTGGAATATTGCACTCCGACTACAT |
| | CAGGAGAAGATGTTCGAGACTTTGCCAAGGTACTAAAAAACAAA |
| | TTTCGAACCAAAAGGTATTTTGCGAAGCATCCCCGAATGGGCTA |
| | CCTGCCAGTGCAGACTGTCTTAGAGGGGGACAACATGGAAACTG |
| | ACACAATTTAG |
| | [SEQ ID NO: 5] |
| SV40- | AACTTGTTTATTGCAGCTTATAATGGTTACAAATAAAGCAATAGC |
| poly(A) tail | ATCACAAATTTCACAAATAAAGCATTTTTTTCACTGC |
| | [SEQ ID NO: 6] |
| 3′ AAV2 | AGGAACCCCTAGTGATGGAGTTGGCCACTCCCTCTCTGCGCGCTC |
| ITR | GCTCGCTCACTGAGGCCGGGCGACCAAAGGTCGCCCGACGCCCG |
| | GGCTTTGCCCGGGCGGCCTCAGTGAGCGAGCGAGCGCGCAG |
| | [SEQ ID NO: 7] |
| SK-CRM4 | TTCTGAGTTCTCTAAGGTCCCTCACTCCCAACTCAGACCCAAGTC |
| enhancer | CTGTCAATTCCCATTCAGTGTCTGATCTCCTTCTTCTCACCTTCCC |
| | CATCTTCCATTTGACCCAAGCTTCCTGAGCACTCCTCCCATTCCC |
| | CTTTTTGGAGTCCTCCTCCTCTCCCAGAACCCAGTAATAAGTGGG |
| | CTCCTCCCTGGCCTGGACCCCCATGGTAACCCTATAAGGCGAGG |
| | CAGCTGCCATCTGAGGCAGGGAGGGGCTGGTGTGGGAGGCTAAG |
| | G |
| | [SEQ ID NO: 8] |
| CMV | CGTTACATAACTTACGGTAAATGGCCCGCCTGGCTGACCGCCCA |
| enhancer | ACGACCCCCGCCCATTGACGTCAATAATGACGTATGTTCCCATAG |
| | TAACGCCAATAGGGACTTTCCATTGACGTCAATGGGTGGAGTAT |
| | TTACGGTAAACTGCCCACTTGGCAGTACATCAAGTGTATCATATG |
| | CCAAGTACGCCCCCTATTGACGTCAATGACGGTAAATGGCCCGC |
| | CTGGCATTATGCCCAGTACATGACCTTATGGGACTTTCCTACTTG |
| | GCAGTACATCTAC |
| | [SEQ ID NO: 9] |

Nucleic acid elements disclosed herein can be ligated together using standard molecular biology techniques to form gene constructs (see, for example, “Molecular Cloning: A Laboratory Manual, 2nd Ed.” (Sambrook et al., 1989), “Current Protocols in Molecular Biology” (Ausubel et al., 1987)).

Gene constructs described herein minimally comprise (from 5′ to 3′): (i) a promoter, and (ii) a transgene. For example, in some embodiments the promoter is an MHCK7 promoter. In certain embodiments, the transgene comprises a nucleic acid encoding μDys. In further embodiments, said gene constructs can comprise one or more additional regulatory element. In one embodiment, a gene construct comprises from 5′ to 3′, a promoter, a transgene, and a SV40 poly (A) tail. In another embodiment, a gene construct comprises from 5′ to 3′, an enhancer, a promoter, a transgene, an SV40 intron and a SV40 poly (A) tail.

In some embodiments, a gene construct comprises (from 5′ to 3′): (i) a promoter, (ii) and SV40 intron, and (iii) a transgene. In certain embodiments, the promoter is an MHCK7 promoter. In certain embodiments, the transgene is a nucleic acid encoding μDys. In further embodiments, said gene constructs can comprise one or more additional regulatory element. In some embodiments, gene constructs described herein comprise a continuous nucleic acid sequence having at least 80%, 85%, 90%, 91%, 92%, 93%, 94%, 95%, 96%, 97%, 98%, 99%, or 100% sequence identity to SEQ ID NO:10. In some embodiments, the gene construct comprises SEQ ID NO:10. In some embodiments, the gene construct consists of SEQ ID NO:10.

Gene Construct 1 (SEQ ID NO: 10)

CCTTCAGATTAAAAATAACTGAGGTAAGGGCCTGGGTAGGGGAGGTGGT
GTGAGACGCTCCTGTCTCTCCTCTATCTGCCCATCGGCCCTTTGGGGAG
GAGGAATGTGCCCAAGGACTAAAAAAAGGCCATGGAGCCAGAGGGGCGA
GGGCAACAGACCTTTCATGGGCAAACCTTGGGGCCCTGCTGTCTAGCAT
GCCCCACTACGGGTCTAGGCTGCCCATGTAAGGAGGCAAGGCCTGGGGA
CACCCGAGATGCCTGGTTATAATTAACCCAGACATGTGGCTGCCCCCCC
CCCCCCAACACCTGCTGCCTCTAAAAATAACCCTGTCCCTGGTGGATCC
CCTGCATGCGAAGATCTTCGAACAAGGCTGTGGGGGACTGAGGGCAGGC
TGTAACAGGCTTGGGGGCCAGGGCTTATACGTGCCTGGGACTCCCAAAG
TATTACTGTTCCATGTTCCCGGCGAAGGGCCAGCTGTCCCCCGCCAGCT
AGACTCAGCACTTAGTTTAGGAACCAGTGAGCAAGTCAGCCCTTGGGGC
AGCCCATACAAGGCCATGGGGCTGGGCAAGCTGCACGCCTGGGTCCGGG
GTGGGCACGGTGCCCGGGCAACGAGCTGAAAGCTCATCTGCTCTCAGGG
GCCCCTCCCTGGGGACAGCCCCTCCTGGCTAGTCACACCCTGTAGGCTC
CTCTATATAACCCAGGGGCACAGGGGCTGCCCTCATTCTACCACCACCT
CCACAGCCTTCAGATTAAAAATAACTGAGGTAAGGGCCTGGGTAGGGGA
GGTGGTGTGAGACGCTCCTGTCTCTCCTCTATCTGCCCATCGGCCCTTT
GGGGAGGAGGAATGTGCCCAAGGACTAAAAAAAGGCCATGGAGCCAGAG
GGGCGAGGGCAACAGACCTTTCATGGGCAAACCTTGGGGCCCTGCTGTC
TAGCATGCCCCACTACGGGTCTAGGCTGCCCATGTAAGGAGGCAAGGCC
TGGGGACACCCGAGATGCCTGGTTATAATTAACCCAGACATGTGGCTGC
CCCCCCCCCCCCAACACCTGCTGCCTCTAAAAATAACCCTGTCCCTGGT
GGATCCCCTGCATGCGAAGATCTTCGAACAAGGCTGTGGGGGACTGAGG
GCAGGCTGTAACAGGCTTGGGGGCCAGGGCTTATACGTGCCTGGGACTC
CCAAAGTATTACTGTTCCATGTTCCCGGCGAAGGGCCAGCTGTCCCCCG
CCAGCTAGACTCAGCACTTAGTTTAGGAACCAGTGAGCAAGTCAGCCCT
TGGGGCAGCCCATACAAGGCCATGGGGCTGGGCAAGCTGCACGCCTGGG
TCCGGGGTGGGCACGGTGCCCGGGCAACGAGCTGAAAGCTCATCTGCTC
TCAGGGGCCCCTCCCTGGGGACAGCCCCTCCTGGCTAGTCACACCCTGT
AGGCTCCTCTATATAACCCAGGGGCACAGGGGCTGCCCTCATTCTACCA
CCACCTCCACAGCACAGACAGACACTCAGGAGCCAGCCAGCCGTAAGTT
TAGTCTTTTTTGTCTTTTATTTCAGGTCCCGGATCCGGTGGTGGTGCAA
ATCAAAGAACTGCTCCTCAGTCGATGTTGCCTTTACTTCTAGATGCTTT
GGTGGGAAGAAGTAGAGGACTGTTATGAAAGAGAAGATGTTCAAAAGAA
AACATTCACAAAATGGGTAAATGCACAATTTTCTAAGTTTGGGAAGCAG
CATATTGAGAACCTCTTCAGTGACCTACAGGATGGGAGGCGCCTCCTAG
ACCTCCTCGAAGGCCTGACAGGGCAAAAACTGCCAAAAGAAAAAGGATC
CACAAGAGTTCATGCCCTGAACAATGTCAACAAGGCACTGCGGGTTTTG
CAGAACAATAATGTTGATTTAGTGAATATTGGAAGTACTGACATCGTAG
ATGGAAATCATAAACTGACTCTTGGTTTGATTTGGAATATAATCCTCCA
CTGGCAGGTCAAAAATGTAATGAAAAATATCATGGCTGGATTGCAACAA
ACCAACAGTGAAAAGATTCTCCTGAGCTGGGTCCGACAATCAACTCGTA
ATTATCCACAGGTTAATGTAATCAACTTCACCACCAGCTGGTCTGATGG
CCTGGCTTTGAATGCTCTCATCCATAGTCATAGGCCAGACCTATTTGAC
TGGAATAGTGTGGTTTGCCAGCAGTCAGCCACACAACGACTGGAACATG
CATTCAACATCGCCAGATATCAATTAGGCATAGAGAAACTACTCGATCC
TGAAGATGTTGATACCACCTATCCAGATAAGAAGTCCATCTTAATGTAC
ATCACATCACTCTTCCAAGTTTTGCCTCAACAAGTGAGCATTGAAGCCA
TCCAGGAAGTGGAAATGTTGCCAAGGCCACCTAAAGTGACTAAAGAAGA
ACATTTTCAGTTACATCATCAAATGCACTATTCTCAACAGATCACGGTC
AGTCTAGCACAGGGATATGAGAGAACTTCTTCCCCTAAGCCTCGATTCA
AGAGCTATGCCTACACACAGGCTGCTTATGTCACCACCTCTGACCCTAC
ACGGAGCCCATTTCCTTCACAGCATTTGGAAGCTCCTGAAGACAAGTCA
TTTGGCAGTTCATTGATGGAGAGTGAAGTAAACCTGGACCGTTATCAAA
CAGCTTTAGAAGAAGTATTATCGTGGCTTCTTTCTGCTGAGGACACATT
GCAAGCACAAGGAGAGATTTCTAATGATGTGGAAGTGGTGAAAGACCAG
TTTCATACTCATGAGGGGTACATGATGGATTTGACAGCCCATCAGGGCC
GGGTTGGTAATATTCTACAATTGGGAAGTAAGCTGATTGGAACAGGAAA
ATTATCAGAAGATGAAGAAACTGAAGTACAAGAGCAGATGAATCTCCTA
AATTCAAGATGGGAATGCCTCAGGGTAGCTAGCATGGAAAAACAAAGCA
ATTTACATAGAGTTTTAATGGATCTCCAGAATCAGAAACTGAAAGAGTT
GAATGACTGGCTAACAAAAACAGAAGAAAGAACAAGGAAAATGGAGGAA
GAGCCTCTTGGACCTGATCTTGAAGACCTAAAACGCCAAGTACAACAAC
ATAAGGTGCTTCAAGAAGATCTAGAACAAGAACAAGTCAGGGTCAATTC
TCTCACTCACATGGTGGTGGTAGTTGATGAATCTAGTGGAGATCACGCA
ACTGCTGCTTTGGAAGAACAACTTAAGGTATTGGGAGATCGATGGGCAA
ACATCTGTAGATGGACAGAAGACCGCTGGGTTCTTTTACAAGACATCCT
TCTCAAATGGCAACGTCTTACTGAAGAACAGTGCCTTTTTAGTGCATGG
CTTTCAGAAAAAGAAGATGCAGTGAACAAGATTCACACAACTGGCTTTA
AAGATCAAAATGAAATGTTATCAAGTCTTCAAAAACTGGCCGTTTTAAA
AGCGGATCTAGAAAAGAAAAAGCAATCCATGGGCAAACTGTATTCACTC
AAACAAGATCTTCTTTCAACACTGAAGAATAAGTCAGTGACCCAGAAGA
CGGAAGCATGGCTGGATAACTTTGCCCGGTGTTGGGATAATTTAGTCCA
AAAACTTGAAAAGAGTACAGCACAGATTTCACAGGCTGTCACCACCACT
CAGCCATCACTAACACAGACAACTGTAATGGAAACAGTAACTACGGTGA
CCACAAGGGAACAGATCCTGGTAAAGCATGCTCAAGAGGAACTTCCACC
ACCACCTCCCCAAAAGAAGAGGCAGATTACTGTGGATCTTGAAAGACTC
CAGGAACTTCAAGAGGCCACGGATGAGCTGGACCTCAAGCTGCGCCAAG
CTGAGGTGATCAAGGGATCCTGGCAGCCCGTGGGCGATCTCCTCATTGA
CTCTCTCCAAGATCACCTCGAGAAAGTCAAGGCACTTCGAGGAGAAATT
GCGCCTCTGAAAGAGAACGTGAGCCACGTCAATGACCTTGCTCGCCAGC
TTACCACTTTGGGCATTCAGCTCTCACCGTATAACCTCAGCACTCTGGA
AGACCTGAACACCAGATGGAAGCTTCTGCAGGTGGCCGTCGAGGACCGA
GTCAGGCAGCTGCATGAAGCCCACAGGGACTTTGGTCCAGCATCTCAGC
ACTTTCTTTCCACGTCTGTCCAGGGTCCCTGGGAGAGAGCCATCTCGCC
AAACAAAGTGCCCTACTATATCAACCACGAGACTCAAACAACTTGCTGG
GACCATCCCAAAATGACAGAGCTCTACCAGTCTTTAGCTGACCTGAATA
ATGTCAGATTCTCAGCTTATAGGACTGCCATGAAACTCCGAAGACTGCA
GAAGGCCCTTTGCTTGGATCTCTTGAGCCTGTCAGCTGCATGTGATGCC
TTGGACCAGCACAACCTCAAGCAAAATGACCAGCCCATGGATATCCTGC
AGATTATTAATTGTTTGACCACTATTTATGACCGCCTGGAGCAAGAGCA
CAACAATTTGGTCAACGTCCCTCTCTGCGTGGATATGTGTCTGAACTGG
CTGCTGAATGTTTATGATACGGGACGAACAGGGAGGATCCGTGTCCTGT
CTTTTAAAACTGGCATCATTTCCCTGTGTAAAGCACATTTGGAAGACAA
GTACAGATACCTTTTCAAGCAAGTGGCAAGTTCAACAGGATTTTGTGAC
CAGCGCAGGCTGGGCCTCCTTCTGCATGATTCTATCCAAATTCCAAGAC
AGTTGGGTGAAGTTGCATCCTTTGGGGGCAGTAACATTGAGCCAAGTGT
CCGGAGCTGCTTCCAATTTGCTAATAATAAGCCAGAGATCGAAGCGGCC
CTCTTCCTAGACTGGATGAGACTGGAACCCCAGTCCATGGTGTGGCTGC
CCGTCCTGCACAGAGTGGCTGCTGCAGAAACTGCCAAGCATCAGGCCAA
ATGTAACATCTGCAAAGAGTGTCCAATCATTGGATTCAGGTACAGGAGT
CTAAAGCACTTTAATTATGACATCTGCCAAAGCTGCTTTTTTTCTGGTC
GAGTTGCAAAAGGCCATAAAATGCACTATCCCATGGTGGAATATTGCAC
TCCGACTACATCAGGAGAAGATGTTCGAGACTTTGCCAAGGTACTAAAA
AACAAATTTCGAACCAAAAGGTATTTTGCGAAGCATCCCCGAATGGGCT
ACCTGCCAGTGCAGACTGTCTTAGAGGGGGACAACATGGAAACTGACAC
AATTTAGAACTTGTTTATTGCAGCTTATAATGGTTACAAATAAAGCAAT
AGCATCACAAATTTCACAAATAAAGCATTTTTTTCACTGC

In some embodiments, a gene construct comprises (from 5′ to 3′): (i) an enhancer, (ii) a promoter, (iii) a transgene, and (iv) a sequence encoding an SV40 poly(A) tail. In certain embodiments, the enhancer is an SK-CRM4 enhancer. In certain embodiments, the promoter is an MHCK7 promoter. In certain embodiments the transgene is a nucleic acid encoding a μDys. In further embodiments, said gene constructs can comprise one or more additional regulatory element. In some embodiments, a gene construct described herein comprises a continuous nucleic acid sequence having at least 80%, 85%, 90%, 91%, 92%, 93%, 94%, 95%, 96%, 97%, 98%, 99%, or 100% sequence identity to SEQ ID NO:11. In some embodiments, the gene construct comprises a nucleic acid sequence of SEQ ID NO: 11. In some embodiments, the gene construct consists of a nucleic acid sequence of SEQ ID NO: 11.

In some embodiments, a vector genome described herein comprises a nucleic acid sequence having at least 80%, 85%, 90%, 91%, 92%, 93%, 94%, 95%, 96%, 97%, 98%, 99%, or 100% sequence identity to SEQ ID NO:12. In some embodiments, the gene construct consists of SEQ ID NO: 12. In some embodiments, the vector genome comprises a nucleic acid sequence of SEQ ID NO:12. In some embodiments, the vector genome consists of a nucleic acid sequence of SEQ ID NO:12.

Gene Construct 2 (SEQ ID NO: 11)
TTCTGAGTTCTCTAAGGTCCCTCACTCCCAACTCAGACCCAAGTCCTGTCAATT
CCCATTCAGTGTCTGATCTCCTTCTTCTCACCTTCCCCATCTTCCATTTGACCCA
AGCTTCCTGAGCACTCCTCCCATTCCCCTTTTTGGAGTCCTCCTCCTCTCCCAGA
ACCCAGTAATAAGTGGGCTCCTCCCTGGCCTGGACCCCCATGGTAACCCTATA
AGGCGAGGCAGCTGCCATCTGAGGCAGGGAGGGGCTGGTGTGGGAGGCTAAG
GCCTTCAGATTAAAAATAACTGAGGTAAGGGCCTGGGTAGGGGAGGTGGTGTG
AGACGCTCCTGTCTCTCCTCTATCTGCCCATCGGCCCTTTGGGGAGGAGGAATG
TGCCCAAGGACTAAAAAAAGGCCATGGAGCCAGAGGGGCGAGGGCAACAGAC
CTTTCATGGGCAAACCTTGGGGCCCTGCTGTCTAGCATGCCCCACTACGGGTCT
AGGCTGCCCATGTAAGGAGGCAAGGCCTGGGGACACCCGAGATGCCTGGTTAT
AATTAACCCAGACATGTGGCTGCCCCCCCCCCCCCAACACCTGCTGCCTCTAAA
AATAACCCTGTCCCTGGTGGATCCCCTGCATGCGAAGATCTTCGAACAAGGCT
GTGGGGGACTGAGGGCAGGCTGTAACAGGCTTGGGGGCCAGGGCTTATACGTG
CCTGGGACTCCCAAAGTATTACTGTTCCATGTTCCCGGCGAAGGGCCAGCTGTC
CCCCGCCAGCTAGACTCAGCACTTAGTTTAGGAACCAGTGAGCAAGTCAGCCC
TTGGGGCAGCCCATACAAGGCCATGGGGCTGGGCAAGCTGCACGCCTGGGTCC
GGGGTGGGCACGGTGCCCGGGCAACGAGCTGAAAGCTCATCTGCTCTCAGGGG
CCCCTCCCTGGGGACAGCCCCTCCTGGCTAGTCACACCCTGTAGGCTCCTCTAT
ATAACCCAGGGGCACAGGGGCTGCCCTCATTCTACCACCACCTCCACAGCACA
GACAGACACTCAGGAGCCAGCCAGCCATGCTTTGGTGGGAAGAAGTAGAGGA
CTGTTATGAAAGAGAAGATGTTCAAAAGAAAACATTCACAAAATGGGTAAATG
CACAATTTTCTAAGTTTGGGAAGCAGCATATTGAGAACCTCTTCAGTGACCTAC
AGGATGGGAGGCGCCTCCTAGACCTCCTCGAAGGCCTGACAGGGCAAAAACT
GCCAAAAGAAAAAGGATCCACAAGAGTTCATGCCCTGAACAATGTCAACAAG
GCACTGCGGGTTTTGCAGAACAATAATGTTGATTTAGTGAATATTGGAAGTACT
GACATCGTAGATGGAAATCATAAACTGACTCTTGGTTTGATTTGGAATATAATC
CTCCACTGGCAGGTCAAAAATGTAATGAAAAATATCATGGCTGGATTGCAACA
AACCAACAGTGAAAAGATTCTCCTGAGCTGGGTCCGACAATCAACTCGTAATT
ATCCACAGGTTAATGTAATCAACTTCACCACCAGCTGGTCTGATGGCCTGGCTT
TGAATGCTCTCATCCATAGTCATAGGCCAGACCTATTTGACTGGAATAGTGTGG
TTTGCCAGCAGTCAGCCACACAACGACTGGAACATGCATTCAACATCGCCAGA
TATCAATTAGGCATAGAGAAACTACTCGATCCTGAAGATGTTGATACCACCTA
TCCAGATAAGAAGTCCATCTTAATGTACATCACATCACTCTTCCAAGTTTTGCC
TCAACAAGTGAGCATTGAAGCCATCCAGGAAGTGGAAATGTTGCCAAGGCCAC
CTAAAGTGACTAAAGAAGAACATTTTCAGTTACATCATCAAATGCACTATTCTC
AACAGATCACGGTCAGTCTAGCACAGGGATATGAGAGAACTTCTTCCCCTAAG
CCTCGATTCAAGAGCTATGCCTACACACAGGCTGCTTATGTCACCACCTCTGAC
CCTACACGGAGCCCATTTCCTTCACAGCATTTGGAAGCTCCTGAAGACAAGTC
ATTTGGCAGTTCATTGATGGAGAGTGAAGTAAACCTGGACCGTTATCAAACAG
CTTTAGAAGAAGTATTATCGTGGCTTCTTTCTGCTGAGGACACATTGCAAGCAC
AAGGAGAGATTTCTAATGATGTGGAAGTGGTGAAAGACCAGTTTCATACTCAT
GAGGGGTACATGATGGATTTGACAGCCCATCAGGGCCGGGTTGGTAATATTCT
ACAATTGGGAAGTAAGCTGATTGGAACAGGAAAATTATCAGAAGATGAAGAA
ACTGAAGTACAAGAGCAGATGAATCTCCTAAATTCAAGATGGGAATGCCTCAG
GGTAGCTAGCATGGAAAAACAAAGCAATTTACATAGAGTTTTAATGGATCTCC
AGAATCAGAAACTGAAAGAGTTGAATGACTGGCTAACAAAAACAGAAGAAAG
AACAAGGAAAATGGAGGAAGAGCCTCTTGGACCTGATCTTGAAGACCTAAAA
CGCCAAGTACAACAACATAAGGTGCTTCAAGAAGATCTAGAACAAGAACAAG
TCAGGGTCAATTCTCTCACTCACATGGTGGTGGTAGTTGATGAATCTAGTGGAG
ATCACGCAACTGCTGCTTTGGAAGAACAACTTAAGGTATTGGGAGATCGATGG
GCAAACATCTGTAGATGGACAGAAGACCGCTGGGTTCTTTTACAAGACATCCT
TCTCAAATGGCAACGTCTTACTGAAGAACAGTGCCTTTTTAGTGCATGGCTTTC
AGAAAAAGAAGATGCAGTGAACAAGATTCACACAACTGGCTTTAAAGATCAA
AATGAAATGTTATCAAGTCTTCAAAAACTGGCCGTTTTAAAAGCGGATCTAGA
AAAGAAAAAGCAATCCATGGGCAAACTGTATTCACTCAAACAAGATCTTCTTT
CAACACTGAAGAATAAGTCAGTGACCCAGAAGACGGAAGCATGGCTGGATAA
CTTTGCCCGGTGTTGGGATAATTTAGTCCAAAAACTTGAAAAGAGTACAGCAC
AGATTTCACAGGCTGTCACCACCACTCAGCCATCACTAACACAGACAACTGTA
ATGGAAACAGTAACTACGGTGACCACAAGGGAACAGATCCTGGTAAAGCATG
CTCAAGAGGAACTTCCACCACCACCTCCCCAAAAGAAGAGGCAGATTACTGTG
GATCTTGAAAGACTCCAGGAACTTCAAGAGGCCACGGATGAGCTGGACCTCAA
GCTGCGCCAAGCTGAGGTGATCAAGGGATCCTGGCAGCCCGTGGGCGATCTCC
TCATTGACTCTCTCCAAGATCACCTCGAGAAAGTCAAGGCACTTCGAGGAGAA
ATTGCGCCTCTGAAAGAGAACGTGAGCCACGTCAATGACCTTGCTCGCCAGCT
TACCACTTTGGGCATTCAGCTCTCACCGTATAACCTCAGCACTCTGGAAGACCT
GAACACCAGATGGAAGCTTCTGCAGGTGGCCGTCGAGGACCGAGTCAGGCAG
CTGCATGAAGCCCACAGGGACTTTGGTCCAGCATCTCAGCACTTTCTTTCCACG
TCTGTCCAGGGTCCCTGGGAGAGAGCCATCTCGCCAAACAAAGTGCCCTACTA
TATCAACCACGAGACTCAAACAACTTGCTGGGACCATCCCAAAATGACAGAGC
TCTACCAGTCTTTAGCTGACCTGAATAATGTCAGATTCTCAGCTTATAGGACTG
CCATGAAACTCCGAAGACTGCAGAAGGCCCTTTGCTTGGATCTCTTGAGCCTGT
CAGCTGCATGTGATGCCTTGGACCAGCACAACCTCAAGCAAAATGACCAGCCC
ATGGATATCCTGCAGATTATTAATTGTTTGACCACTATTTATGACCGCCTGGAG
CAAGAGCACAACAATTTGGTCAACGTCCCTCTCTGCGTGGATATGTGTCTGAAC
TGGCTGCTGAATGTTTATGATACGGGACGAACAGGGAGGATCCGTGTCCTGTC
TTTTAAAACTGGCATCATTTCCCTGTGTAAAGCACATTTGGAAGACAAGTACAG
ATACCTTTTCAAGCAAGTGGCAAGTTCAACAGGATTTTGTGACCAGCGCAGGC
TGGGCCTCCTTCTGCATGATTCTATCCAAATTCCAAGACAGTTGGGTGAAGTTG
CATCCTTTGGGGGCAGTAACATTGAGCCAAGTGTCCGGAGCTGCTTCCAATTTG
CTAATAATAAGCCAGAGATCGAAGCGGCCCTCTTCCTAGACTGGATGAGACTG
GAACCCCAGTCCATGGTGTGGCTGCCCGTCCTGCACAGAGTGGCTGCTGCAGA
AACTGCCAAGCATCAGGCCAAATGTAACATCTGCAAAGAGTGTCCAATCATTG
GATTCAGGTACAGGAGTCTAAAGCACTTTAATTATGACATCTGCCAAAGCTGC
TTTTTTTCTGGTCGAGTTGCAAAAGGCCATAAAATGCACTATCCCATGGTGGAA
TATTGCACTCCGACTACATCAGGAGAAGATGTTCGAGACTTTGCCAAGGTACT
AAAAAACAAATTTCGAACCAAAAGGTATTTTGCGAAGCATCCCCGAATGGGCT
ACCTGCCAGTGCAGACTGTCTTAGAGGGGGACAACATGGAAACTGACACAATT
TAGAACTTGTTTATTGCAGCTTATAATGGTTACAAATAAAGCAATAGCATCACA
AATTTCACAAATAAAGCATTTTTTTCACTGC
Exemplary vector genome sequence of the present invention (SEQ ID NO: 12)
CTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTG
GCCAACTCCATCACTAGGGGTTCCTTGTAGTTAATGATTAACCCGCCATGCTAC
TTATCTACGTAGCAAGCTAGCGTACCTCGCGAATGCATCTAGATTCGCGACGTT
ACATAACTTACGGTAAATGGCCCGCCTGGCTGACCGCCCAACGACCCCCGCCC
ATTGACGTCAATAATGACGTATGTTCCCATAGTAACGCCAATAGGGACTTTCCA
TTGACGTCAATGGGTGGAGTATTTACGGTAAACTGCCCACTTGGCAGTACATC
AAGTGTATCATATGCCAAGTACGCCCCCTATTGACGTCAATGACGGTAAATGG
CCCGCCTGGCATTATGCCCAGTACATGACCTTATGGGACTTTCCTACTTGGCAG
TACATCTACTCGAGGCCACGTTCTGCTTCACTCTCCCCATCTCCCCCCCCTCCCC
ACCCCCAATTTTGTATTTATTTATTTTTTAATTATTTTGTGCAGCGATGGGGGCG
GGGGGGGGGGGGGGGCGCGCGCCAGGCGGGGCGGGGCGGGGCGAGGGGCGG
GGCGGGGCGAGGCGGAGAGGTGCGGCGGCAGCCAATCAGAGCGGCGCGCTCC
GAAAGTTTCCTTTTATGGCGAGGCGGCGGCGGCGGCGGCCCTATAAAAAGCGA
AGCGCGCGGCGGGCGGGAGGTAAGTTTAGTCTTTTTGTCTTTTATTTCAGGTCC
CGGATCCGGTGGTGGTGCAAATCAAAGAACTGCTCCTCAGTGGATGTTGCCTTT
ACTTCTAGATGCTTTGGTGGGAAGAAGTAGAGGACTGTTATGAAAGAGAAGAT
GTTCAAAAGAAAACATTCACAAAATGGGTAAATGCACAATTTTCTAAGTTTGG
GAAGCAGCATATTGAGAACCTCTTCAGTGACCTACAGGATGGGAGGCGCCTCC
TAGACCTCCTCGAAGGCCTGACAGGGCAAAAACTGCCAAAAGAAAAAGGATC
CACAAGAGTTCATGCCCTGAACAATGTCAACAAGGCACTGCGGGTTTTGCAGA
ACAATAATGTTGATTTAGTGAATATTGGAAGTACTGACATCGTAGATGGAAAT
CATAAACTGACTCTTGGTTTGATTTGGAATATAATCCTCCACTGGCAGGTCAAA
AATGTAATGAAAAATATCATGGCTGGATTGCAACAAACCAACAGTGAAAAGAT
TCTCCTGAGCTGGGTCCGACAATCAACTCGTAATTATCCACAGGTTAATGTAAT
CAACTTCACCACCAGCTGGTCTGATGGCCTGGCTTTGAATGCTCTCATCCATAG
TCATAGGCCAGACCTATTTGACTGGAATAGTGTGGTTTGCCAGCAGTCAGCCA
CACAACGACTGGAACATGCATTCAACATCGCCAGATATCAATTAGGCATAGAG
AAACTACTCGATCCTGAAGATGTTGATACCACCTATCCAGATAAGAAGTCCAT
CTTAATGTACATCACATCACTCTTCCAAGTTTTGCCTCAACAAGTGAGCATTGA
AGCCATCCAGGAAGTGGAAATGTTGCCAAGGCCACCTAAAGTGACTAAAGAA
GAACATTTTCAGTTACATCATCAAATGCACTATTCTCAACAGATCACGGTCAGT
CTAGCACAGGGATATGAGAGAACTTCTTCCCCTAAGCCTCGATTCAAGAGCTA
TGCCTACACACAGGCTGCTTATGTCACCACCTCTGACCCTACACGGAGCCCATT
TCCTTCACAGCATTTGGAAGCTCCTGAAGACAAGTCATTTGGCAGTTCATTGAT
GGAGAGTGAAGTAAACCTGGACCGTTATCAAACAGCTTTAGAAGAAGTATTAT
CGTGGCTTCTTTCTGCTGAGGACACATTGCAAGCACAAGGAGAGATTTCTAAT
GATGTGGAAGTGGTGAAAGACCAGTTTCATACTCATGAGGGGTACATGATGGA
TTTGACAGCCCATCAGGGCCGGGTTGGTAATATTCTACAATTGGGAAGTAAGC
TGATTGGAACAGGAAAATTATCAGAAGATGAAGAAACTGAAGTACAAGAGCA
GATGAATCTCCTAAATTCAAGATGGGAATGCCTCAGGGTAGCTAGCATGGAAA
AACAAAGCAATTTACATAGAGTTTTAATGGATCTCCAGAATCAGAAACTGAAA
GAGTTGAATGACTGGCTAACAAAAACAGAAGAAAGAACAAGGAAAATGGAGG
AAGAGCCTCTTGGACCTGATCTTGAAGACCTAAAACGCCAAGTACAACAACAT
AAGGTGCTTCAAGAAGATCTAGAACAAGAACAAGTCAGGGTCAATTCTCTCAC
TCACATGGTGGTGGTAGTTGATGAATCTAGTGGAGATCACGCAACTGCTGCTTT
GGAAGAACAACTTAAGGTATTGGGAGATCGATGGGCAAACATCTGTAGATGG
ACAGAAGACCGCTGGGTTCTTTTACAAGACATCCTTCTCAAATGGCAACGTCTT
ACTGAAGAACAGTGCCTTTTTAGTGCATGGCTTTCAGAAAAAGAAGATGCAGT
GAACAAGATTCACACAACTGGCTTTAAAGATCAAAATGAAATGTTATCAAGTC
TTCAAAAACTGGCCGTTTTAAAAGCGGATCTAGAAAAGAAAAAGCAATCCATG
GGCAAACTGTATTCACTCAAACAAGATCTTCTTTCAACACTGAAGAATAAGTC
AGTGACCCAGAAGACGGAAGCATGGCTGGATAACTTTGCCCGGTGTTGGGATA
ATTTAGTCCAAAAACTTGAAAAGAGTACAGCACAGATTTCACAGGCTGTCACC
ACCACTCAGCCATCACTAACACAGACAACTGTAATGGAAACAGTAACTACGGT
GACCACAAGGGAACAGATCCTGGTAAAGCATGCTCAAGAGGAACTTCCACCA
CCACCTCCCCAAAAGAAGAGGCAGATTACTGTGGATCTTGAAAGACTCCAGGA
ACTTCAAGAGGCCACGGATGAGCTGGACCTCAAGCTGCGCCAAGCTGAGGTGA
TCAAGGGATCCTGGCAGCCCGTGGGCGATCTCCTCATTGACTCTCTCCAAGATC
ACCTCGAGAAAGTCAAGGCACTTCGAGGAGAAATTGCGCCTCTGAAAGAGAA
CGTGAGCCACGTCAATGACCTTGCTCGCCAGCTTACCACTTTGGGCATTCAGCT
CTCACCGTATAACCTCAGCACTCTGGAAGACCTGAACACCAGATGGAAGCTTC
TGCAGGTGGCCGTCGAGGACCGAGTCAGGCAGCTGCATGAAGCCCACAGGGA
CTTTGGTCCAGCATCTCAGCACTTTCTTTCCACGTCTGTCCAGGGTCCCTGGGA
GAGAGCCATCTCGCCAAACAAAGTGCCCTACTATATCAACCACGAGACTCAAA
CAACTTGCTGGGACCATCCCAAAATGACAGAGCTCTACCAGTCTTTAGCTGAC
CTGAATAATGTCAGATTCTCAGCTTATAGGACTGCCATGAAACTCCGAAGACT
GCAGAAGGCCCTTTGCTTGGATCTCTTGAGCCTGTCAGCTGCATGTGATGCCTT
GGACCAGCACAACCTCAAGCAAAATGACCAGCCCATGGATATCCTGCAGATTA
TTAATTGTTTGACCACTATTTATGACCGCCTGGAGCAAGAGCACAACAATTTGG
TCAACGTCCCTCTCTGCGTGGATATGTGTCTGAACTGGCTGCTGAATGTTTATG
ATACGGGACGAACAGGGAGGATCCGTGTCCTGTCTTTTAAAACTGGCATCATT
TCCCTGTGTAAAGCACATTTGGAAGACAAGTACAGATACCTTTTCAAGCAAGT
GGCAAGTTCAACAGGATTTTGTGACCAGCGCAGGCTGGGCCTCCTTCTGCATG
ATTCTATCCAAATTCCAAGACAGTTGGGTGAAGTTGCATCCTTTGGGGGCAGTA
ACATTGAGCCAAGTGTCCGGAGCTGCTTCCAATTTGCTAATAATAAGCCAGAG
ATCGAAGCGGCCCTCTTCCTAGACTGGATGAGACTGGAACCCCAGTCCATGGT
GTGGCTGCCCGTCCTGCACAGAGTGGCTGCTGCAGAAACTGCCAAGCATCAGG
CCAAATGTAACATCTGCAAAGAGTGTCCAATCATTGGATTCAGGTACAGGAGT
CTAAAGCACTTTAATTATGACATCTGCCAAAGCTGCTTTTTTTCTGGTCGAGTT
GCAAAAGGCCATAAAATGCACTATCCCATGGTGGAATATTGCACTCCGACTAC
ATCAGGAGAAGATGTTCGAGACTTTGCCAAGGTACTAAAAAACAAATTTCGAA
CCAAAAGGTATTTTGCGAAGCATCCCCGAATGGGCTACCTGCCAGTGCAGACT
GTCTTAGAGGGGGACAACATGGAAACTGACACAATTTAGAACTTGTTTATTGC
AGCTTATAATGGTTACAAATAAAGCAATAGCATCACAAATTTCACAAATAAAG
CATTTTTTTCACTGCTCGCGAATCGGATCCCCTCGAGTTCTACGTAGATAAGTA
GCATGGCGGGTTAATCATTAACTACAAGGAACCCCTAGTGATGGAGTTGGCCA
CTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAG

### AAV Vector Backbones

In some embodiments, an AAV vector genome of the present invention can be assembled by inserting a gene construct, as described herein, into an appropriate adenovirus plasmid backbone using standard molecular biology techniques (see, for example, Sambrook et al. (1989). “Molecular Cloning: A Laboratory Manual, 2nd Ed.”; Ausubel et al. (1987). “Current Protocols in Molecular Biology”). The adenovirus plasmid backbone, in one embodiment, comprises the 5′ ITR and 3′ ITR sequences described herein. The gene construct is inserted into the adenovirus plasmid backbone between the ITR sequences, downstream of the 5′ ITR sequence and upstream of the 3′ ITR sequence.

For example, in one embodiment, an AAV vector genome of the present invention can be assembled by inserting a gene construct comprising the sequence of SEQ ID NO: 10 into an adenovirus plasmid backbone comprising the sequence of SEQ ID NO: 13.

In another embodiment, an AAV vector genome of the present invention can be assembled by inserting a gene construct comprising the sequence of SEQ ID NO:11 into an adenovirus plasmid backbone comprising the sequence of SEQ ID NO:13.

In some embodiments, the adenovirus plasmid backbone of the present invention comprises a nucleic acid sequence having at least 8000, 8500, 9000, 9100, 9200, 9300, 9400, 9500, 96%, 97%, 98%, 99%, or 100% sequence identity to SEQ ID NO: 13. In some embodiments, the adenovirus plasmid backbone comprises a nucleic acid sequence of SEQ ID NO: 13. In some embodiments, the adenovirus plasmid backbone consists of a nucleic acid sequence of SEQ ID NO: 13, provided below.

Exemplary Adenovirus Plasmid Backbone
(SEQ ID NO: 13)

AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAGCCTTAATTAACCTAAGG
AAAATGAAGTGAAGTTCCTATACTTTCTAGAGAATAGGAACTTCTATAG
TGAGTCGAATAAGGGCGACACAAAATTTATTCTAAATGCATAATAAATA
CTGATAACATCTTATAGTTTGTATTATATTTTGTATTATCGTTGACATG
TATAATTTTGATATCAAAAACTGATTTTCCCTTTATTATTTTCGAGATT
TATTTTCTTAATTCTCTTTAACAAACTAGAAATATTGTATATACAAAAA
ATCATAAATAATAGATGAATAGTTTAATTATAGGTGTTCATCAATCGAA
AAAGCAACGTATCTTATTTAAAGTGCGTTGCTTTTTTCTCATTTATAAG
GTTAAATAATTCTCATATATCAAGCAAAGTGACAGGCGCCCTTAAATAT
TCTGACAAATGCTCTTTCCCTAAACTCCCCCCATAAAAAAACCCGCCGA
AGCGGGTTTTTACGTTATTTGCGGATTAACGATTACTCGTTATCAGAAC
CGCCCAGGGGGCCCGAGCTTAACCTTTTTATTTGGGGGAGAGGGAAGTC
ATGAAAAAACTAACCTTTGAAATTCGATCTCCAGCACATCAGCAAAACG
CTATTCACGCAGTACAGCAAATCCTTCCAGACCCAACCAAACCAATCGT
AGTAACCATTCAGGAACGCAACCGCAGCTTAGACCAAAACAGGAAGCTA
TGGGCCTGCTTAGGTGACGTCTCTCGTCAGGTTGAATGGCATGGTCGCT
GGCTGGATGCAGAAAGCTGGAAGTGTGTGTTTACCGCAGCATTAAAGCA
GCAGGATGTTGTTCCTAACCTTGCCGGGAATGGCTTTGTGGTAATAGGC
CAGTCAACCAGCAGGATGCGTGTAGGCGAATTTGCGGAGCTATTAGAGC
TTATACAGGCATTCGGTACAGAGCGTGGCGTTAAGTGGTCAGACGAAGC
GAGACTGGCTCTGGAGTGGAAAGCGAGATGGGGAGACAGGGCTGCATGA
TAAATGTCGTTAGTTTCTCCGGTGGCAGGACGTCAGCATATTTGCTCTG
GCTAATGGAGCAAAAGCGACGGGCAGGTAAAGACGTGCATTACGTTTTC
ATGGATACAGGTTGTGAACATCCAATGACATATCGGTTTGTCAGGGAAG
TTGTGAAGTTCTGGGATATACCGCTCACCGTATTGCAGGTTGATATCAA
CCCGGAGCTTGGACAGCCAAATGGTTATACGGTATGGGAACCAAAGGAT
ATTCAGACGCGAATGCCTGTTCTGAAGCCATTTATCGATATGGTAAAGA
AATATGGCACTCCATACGTCGGCGGCGCGTTCTGCACTGACAGATTAAA
ACTCGTTCCCTTCACCAAATACTGTGATGACCATTTCGGGCGAGGGAAT
TACACCACGTGGATTGGCATCAGAGCTGATGAACCGAAGCGGCTAAAGC
CAAAGCCTGGAATCAGATATCTTGCTGAACTGTCAGACTTTGAGAAGGA
AGATATCCTCGCATGGTGGAAGCAACAACCATTCGATTTGCAAATACCG
GAACATCTCGGTAACTGCATATTCTGCATTAAAAAATCAACGCAAAAAA
TCGGACTTGCCTGCAAAGATGAGGAGGGATTGCAGCGTGTTTTTAATGA
GGTCATCACGGGATCCCATGTGCGTGACGGACATCGGGAAACGCCAAAG
GAGATTATGTACCGAGGAAGAATGTCGCTGGACGGTATCGCGAAAATGT
ATTCAGAAAATGATTATCAAGCCCTGTATCAGGACATGGTACGAGCTAA
AAGATTCGATACCGGCTCTTGTTCTGAGTCATGCGAAATATTTGGAGGG
CAGCTTGATTTCGACTTCGGGAGGGAAGCTGCATGATGCGATGTTATCG
GTGCGGTGAATGCAAAGAAGATAACCGCTTCCGACCAAATCAACCTTAC
TGGAATCGATGGTGTCTCCGGTGTGAAAGAACACCAACAGGGGTGTTAC
CACTACCGCAGGAAAAGGAGGACGTGTGGCGAGACAGCGACGAAGTATC
ACCGACATAATCTGCGAAAACTGCAAATACCTTCCAACGAAACGCACCA
GAAATAAACCCAAGCCAATCCCAAAAGAATCTGACGTAAAAACCTTCAA
CTACACGGCTCACCTGTGGGATATCCGGTGGCTAAGACGTCGTGCGAGG
AAAACAAGGTGATTGACCAAAATCGAAGTTACGAACAAGAAAGCGTCGA
GCGAGCTTTAACGTGCGCTAACTGCGGTCAGAAGCTGCATGTGCTGGAA
GTTCACGTGTGTGAGCACTGCTGCGCAGAACTGATGAGCGATCCGAATA
GCTCGATGCACGAGGAAGAAGATGATGGCTAAACCAGCGCGAAGACGAT
GTAAAAACGATGAATGCCGGGAATGGTTTCACCCTGCATTCGCTAATCA
GTGGTGGTGCTCTCCAGAGTGTGGAACCAAGATAGCACTCGAACGACGA
AGTAAAGAACGCGAAAAAGCGGAAAAAGCAGCAGAGAAGAAACGACGAC
GAGAGGAGCAGAAACAGAAAGATAAACTTAAGATTCGAAAACTCGCCTT
AAAGCCCCGCAGTTACTGGATTAAACAAGCCCAACAAGCCGTAAACGCC
TTCATCAGAGAAAGAGACCGCGACTTACCATGTATCTCGTGCGGAACGC
TCACGTCTGCTCAGTGGGATGCCGGACATTACCGGACAACTGCTGCGGC
ACCTCAACTCCGATTTAATGAACGCAATATTCACAAGCAATGCGTGGTG
TGCAACCAGCACAAAAGCGGAAATCTCGTTCCGTATCGCGTCGAACTGA
TTAGCCGCATCGGGCAGGAAGCAGTAGACGAAATCGAATCAAACCATAA
CCGCCATCGCTGGACTATCGAAGAGTGCAAGGCGATCAAGGCAGAGTAC
CAACAGAAACTCAAAGACCTGCGAAATAGCAGAAGTGAGGCCGCATGAC
GTTCTCAGTAAAAACCATTCCAGACATGCTCGTTGAAGCATACGGAAAT
CAGACAGAAGTAGCACGCAGACTGAAATGTAGTCGCGGTACGGTCAGAA
AATACGTTGATGATAAAGACGGGAAAATGCACGCCATCGTCAACGACGT
TCTCATGGTTCATCGCGGATGGAGTGAAAGAGATGCGCTATTACGAAAA
AATTGATGGCAGCAAATACCGAAATATTTGGGTAGTTGGCGATCTGCAC
GGATGCTACACGAACCTGATGAACAAACTGGATACGATTGGATTCGACA
ACAAAAAAGACCTGCTTATCTCGGTGGGCGATTTGGTTGATCGTGGTGC
AGAGAACGTTGAATGCCTGGAATTAATCACATTCCCCTGGTTCAGAGCT
GTACGTGGAAACCATGAGCAAATGATGATTGATGGCTTATCAGAGCGTG
GAAACGTTAATCACTGGCTGCTTAATGGCGGTGGCTGGTTCTTTAATCT
CGATTACGACAAAGAAATTCTGGCTAAAGCTCTTGCCCATAAAGCAGAT
GAACTTCCGTTAATCATCGAACTGGTGAGCAAAGATAAAAAATATGTTA
TCTGCCACGCCGATTATCCCTTTGACGAATACGAGTTTGGAAAGCCAGT
TGATCATCAGCAGGTAATCTGGAACCGCGAACGAATCAGCAACTCACAA
AACGGGATCGTGAAAGAAATCAAAGGCGCGGACACGTTCATCTTTGGTC
ATACGCCAGCAGTGAAACCACTCAAGTTTGCCAACCAAATGTATATCGA
TACCGGCGCAGTGTTCTGCGGAAACCTAACATTGATTCAGGTACAGGGA
GAAGGCGCATGAGACTCGAAAGCGTAGCTAAATTTCATTCGCCAAAAAG
CCCGATGATGAGCGACTCACCACGGGCCACGGCTTCTGACTCTCTTTCC
GGTACTGATGTGATGGCTGCTATGGGGATGGCGCAATCACAAGCCGGAT
TCGGTATGGCTGCATTCTGCGGTAAGCACGAACTCAGCCAGAACGACAA
ACAAAAGGCTATCAACTATCTGATGCAATTTGCACACAAGGTATCGGGG
AAATACCGTGGTGTGGCAAAGCTTGAAGGAAATACTAAGGCAAAGGTAC
TGCAAGTGCTCGCAACATTCGCTTATGCGGATTATTGCCGTAGTGCCGC
GACGCCGGGGGCAAGATGCAGAGATTGCCATGGTACAGGCCGTGCGGTT
GATATTGCCAAAACAGAGCTGTGGGGGAGAGTTGTCGAGAAAGAGTGCG
GAAGATGCAAAGGCGTCGGCTATTCAAGGATGCCAGCAAGCGCAGCATA
TCGCGCTGTGACGATGCTAATCCCAAACCTTACCCAACCCACCTGGTCA
CGCACTGTTAAGCCGCTGTATGACGCTCTGGTGGTGCAATGCCACAAAG
AAGAGTCAATCGCAGACAACATTTTGAATGCGGTCACACGTTAGCAGCA
TGATTGCCACGGATGGCAACATATTAACGGCATGATATTGACTTATTGA
ATAAAATTGGGTAAATTTGACTCAACGATGGGTTAATTCGCTCGTTGTG
GTAGTGAGATGAAAAGAGGCGGCGCTTACTACCGATTCCGCCTAGTTGG
TCACTTCGACGTATCGTCTGGAACTCCAACCATCGCAGGCAGAGAGGTC
TGCAAAATGCAATCCCGAAACAGTTCGCAGGTAATAGTTAGAGCCTGCA
TAACGGTTTCGGGATTTTTTATATCTGCACAACAGGTAAGAGCATTGAG
TCGATAATCGTGAAGAGTCGGCGAGCCTGGTTAGCCAGTGCTCTTTCCG
TTGTGCTGAATTAAGCGAATACCGGAAGCAGAACCGGATCACCAAATGC
GTACAGGCGTCATCGCCGCCCAGCAACAGCACAACCCAAACTGAGCCGT
AGCCACTGTCTGTCCTGAATTCATTAGTAATAGTTACGCTGCGGCCTTT
TACACATGACCTTCGTGAAAGCGGGTGGCAGGAGGTCGCGCTAACAACC
TCCTGCCGTTTTGCCCGTGCATATCGGTCACGAACAAATCTGATTACTA
AACACAGTAGCCTGGATTTGTTCTATCAGTAATCGACCTTATTCCTAAT
TAAATAGAGCAAATCCCCTTATTGGGGGTAAGACATGAAGATGCCAGAA
AAACATGACCTGTTGGCCGCCATTCTCGCGGCAAAGGAACAAGGCATCG
GGGCAATCCTTGCGTTTGCAATGGCGTACCTTCGCGGCAGATATAATGG
CGGTGCGTTTACAAAAACAGTAATCGACGCAACGATGTGCGCCATTATC
GCCTGGTTCATTCGTGACCTTCTCGACTTCGCCGGACTAAGTAGCAATC
TCGCTTATATAACGAGCGTGTTTATCGGCTACATCGGTACTGACTCGAT
TGGTTCGCTTATCAAACGCTTCGCTGCTAAAAAAGCCGGAGTAGAAGAT
GGTAGAAATCAATAATCAACGTAAGGCGTTCCTCGATATGCTGGCGTGG
TCGGAGGGAACTGATAACGGACGTCAGAAAACCAGAAATCATGGTTATG
ACGTCATTGTAGGCGGAGAGCTATTTACTGATTACTCCGATCACCCTCG
CAAACTTGTCACGCTAAACCCAAAACTCAAATCAACAGGCGCTTAAGAC
TGGCCGTCGTTTTACAACACAGAAAGAGTTTGTAGAAACGCAAAAAGGC
CATCCGTCAGGGGCCTTCTGCTTAGTTTGATGCCTGGCAGTTCCCTACT
CTCGCCTTCCGCTTCCTCGCTCACTGACTCGCTGCGCTCGGTCGTTCGG
CTGCGGCGAGCGGTATCAGCTCACTCAAAGGCGGTAATACGGTTATCCA
CAGAATCAGGGGATAACGCAGGAAAGAACATGTGAGCAAAAGGCCAGCA
AAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGT
GGCGAAACCCGACAGGACTATAAAGATACCAGGCGTTTCCCCCTGGAAG
CTCCCTCGTGCGCTCTCCTGTTCCGACCCTGCCGCTTACCGGATACCTG
TCCGCCTTTCTCCCTTCGGGAAGCGTGGCGCTTTCTCATAGCTCACGCT
GTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTAT
CGTCTTGAGTCCAACCCGGTAAGACACGACTTATCGCCACTGGCAGCAG
CCACTGGTAACAGGATTAGCAGAGCGAGGTATGTAGGCGGTGCTACAGA
GTTCTTGAAGTGGTGGGCTAACTACGGCTACACTAGAAGAACAGTATTT
GGTATCTGCGCTCTGCTGAAGCCAGTTACCTTCGGAAAAAGAGTTGGTA
GCTCTTGATCCGGCAAACAAACCACCGCTGGTAGCGGTGGTTTTTTTGT
TTGCAAGCAGCAGATTACGCGCAGAAAAAAAGGATCTCAAGAAGATCCT
TTGATCTTTTCTACGGGGTCTGACGCTCAGTGGAACGACGCGCGCGTAA
CTCACGTTAAGGGATTTTGGTCATGAGCTTGCGCCGTCCCGTCAAGTCA
GCGTAATGCTCTGCTTTTAGAAAAACTCATCGAGCATCAAATGAAACTG
CAATTTATTCATATCAGGATTATCAATACCATATTTTTGAAAAAGCCGT
TTCTGTAATGAAGGAGAAAACTCACCGAGGCAGTTCCATAGGATGGCAA
GATCCTGGTATCGGTCTGCGATTCCGACTCGTCCAACATCAATACAACC
TATTAATTTCCCCTCGTCAAAAATAAGGTTATCAAGTGAGAAATCACCA
TGAGTGACGACTGAATCCGGTGAGAATGGCAAAAGTTTATGCATTTCTT
TCCAGACTTGTTCAACAGGCCAGCCATTACGCTCGTCATCAAAATCACT
CGCATCAACCAAACCGTTATTCATTCGTGATTGCGCCTGAGCGAGGCGA
AATACGCGATCGCTGTTAAAAGGACAATTACAAACAGGAATCGAGTGCA
ACCGGCGCAGGAACACTGCCAGCGCATCAACAATATTTTCACCTGAATC
AGGATATTCTTCTAATACCTGGAACGCTGTTTTTCCGGGGATCGCAGTG
GTGAGTAACCATGCATCATCAGGAGTACGGATAAAATGCTTGATGGTCG
GAAGTGGCATAAATTCCGTCAGCCAGTTTAGTCTGACCATCTCATCTGT
AACATCATTGGCAACGCTACCTTTGCCATGTTTCAGAAACAACTCTGGC
GCATCGGGCTTCCCATACAAGCGATAGATTGTCGCACCTGATTGCCCGA
CATTATCGCGAGCCCATTTATACCCATATAAATCAGCATCCATGTTGGA
ATTTAATCGCGGCCTCGACGTTTCCCGTTGAATATGGCTCATATTCTTC
CTTTTTCAATATTATTGAAGCATTTATCAGGGTTATTGTCTCATGAGCG
GATACATATTTGAATGTATTTAGAAAAATAAACAAATAGGGGTCAGTGT
TACAACCAATTAACCAATTCTGAACATTATCGCGAGCCCATTTATACCT
GAATATGGCTCATAACACCCCTTGTTTGCCTGGCGGCAGTAGCGCGGTG
GTCCCACCTGACCCCATGCCGAACTCAGAAGTGAAACGCCGTAGCGCCG
ATGGTAGTGTGGGGACTCCCCATGCGAGAGTAGGGAACTGCCAGGCATC
AAATAAAACGAAAGGCTCAGTCGAAAGACTGGGCCTTTCGCCCGGGCTA
ATTAGGGGGTGTCGCCCTTATTCGACTCTATAGTGAAGTTCCTATTCTC
TAGAAAGTATAGGAACTTCTGAAGTGGGGTCGACTTAATTAAGGCTGCG
CGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAG
TGGCCAACTCCATCACTAGGGGTTCCT

### Adeno-Associated Viruses (AAV) Particle Production

AAV particles can be produced by any standard method (see, for example, WO 2001/083692; Masic et al. 2014. Molecular Therapy, 22(11):1900-1909; Carter, 1992, Current Opinions in Biotechnology, 1533-539; Muzyczka, 1992, Curr. Topics in Microbial, and Immunol., 158:97-129); Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al, J. Virol, 62: 1963 (1988); and Lebkowski et al, Mol. Cell. Biol, 7:349 (1988). Samulski et al, J. Virol., 63:3822-3828 (1989); U.S. Pat. No. 5,173,414; WO 95/13365; U.S. Pat. No. 5,658,776; WO95/13392; WO 96/17947; PCT/US98/18600; WO 97/09441 (PCT/US 96/14423); WO 97/08298 (PCT/US96/13872); WO 97/21825 (PCT/US96/20777); WO 97/06243 (PCT/FR96/01064); WO 99/11764; Perrin et al. Vaccine 13: 1244-1250 (1995); Paul et al. Human Gene Therapy 4:609-615 (1993); Clark et al. Gene Therapy 3: 1124-1132 (1996); U.S. Pat. Nos. 5,786,211; 5,871,982; and 6,258,595, herein incorporated by reference in their entireties). For example, in some embodiments, AAV vector genomes described herein can be transformed into Escherichia coli to scale-up DNA production, purified using any standard method (for example, a Maxi-Prep K, Thermo Scientific), and verified by restriction digest or sequencing. Purified AAV vector genomes can then be transfected using a standard method (e.g., calcium phosphate transfection, polyethyleneimine, electroporation, and the like) into an appropriate packaging cell line (e.g., HEK293, HeLa, or PerC.6, MRC-5, WI-38, Vera, and FRhL-2 cells) in combination with a plasmid comprising AAV rep and AAV cap genes, and an AAV helper plasmid. The AAV rep and cap genes may be from any AAV serotype and may be the same or different from that of the recombinant AAV vector ITRs including, but not limited to, AAV1, AAV2, AAV3, AAV4, AAV5, AAV6, AAV7, AAVrh.74, AAV8, AAV9, AAV10, AAV11, AAV12, and AAV13. In certain embodiments, AAV particles described herein comprise AAV rep and cap genes derived from AAV2 and AAV9, respectively.

In some embodiments, AAV particles described herein can be harvested from packaging cells and purified by methods standard in the art (e.g., Clark et al, Hum. Gene Ther., 10(6): 1031-1039 (1999); Schenpp and Clark, Methods Mol. Med., 69 427-443 (2002); U.S. Pat. No. 6,566,118 and WO 98/09657, incorporated herein in their entirety by reference) such as by cesium chloride ultracentrifugation gradient or column chromatography.

### Pharmaceutical Compositions

The pharmaceutical compositions provided herein are intrathecal pharmaceutical compositions, i.e., are intended for delivery via the intrathecal route. The intrathecal composition comprises an effective amount of an AAV particle encapsidating a μDys transgene, as described herein. In some embodiments, pharmaceutical compositions disclosed herein comprise an AAV particle of the present invention and a pharmaceutically acceptable carrier and, optionally, other medicinal agents, pharmaceutical agents, stabilizing agents, buffers, carriers, adjuvants, diluents, etc. By “pharmaceutically acceptable” it is meant a material that is not toxic or otherwise undesirable, i.e., the material may be administered to a subject without causing any undesirable biological effects. The pharmaceutical composition is an intrathecal pharmaceutical composition. The effective amount of the AAV particle comprises a lower dose of vector genomes compared to an IV AAV pharmaceutical composition comprising the same vector genome components, or the same transgene. For example, in one embodiment, the effective amount of the AAV particle described herein is about 90% or less vector genomes than the effective amount of an IV composition comprising the same AAV particle or the same micro-dystrophin transgene.

In some embodiments, pharmaceutical compositions provided herein comprise sterile aqueous and non-aqueous injection solutions, which are optionally isotonic with the blood of the subject to whom the pharmaceutical composition is to be delivered. Pharmaceutical compositions can contain antioxidants, buffers, bacteriostats and solutes, which render the composition isotonic with the blood of the intended subject to be administered. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In some embodiments pharmaceutical compositions comprise pharmaceutically acceptable vehicles and can include sodium chloride solution, Ringer's dextrose, dextrose and sodium chloride, lactated Ringer's, or fixed oils. Preservatives and other additives may also be present such as, for example, antimicrobials, antioxidants, chelating agents, and inert gases and the like.

In some embodiments, pharmaceutical compositions can be presented in unit/dose or multi-dose containers, for example, in sealed ampoules and vials, and can be stored in a freeze-dried (lyophilized) condition requiring only the addition of the sterile liquid carrier, for example, saline or water-for-injection immediately prior to use.

In some embodiments, pharmaceutical compositions disclosed herein can be alternatively formulated for IV, intramuscular, or intracerebroventricular (ICV) administration.

### Methods of Treatment

One aspect of the present invention relates to a method of treating a dystrophinopathy in a subject in need thereof, comprising intrathecally administering in a single dose, an intrathecal composition comprising an effective amount of an AAV particle described herein. The methods can be employed for preferentially delivering the AAV particle to cardiac and/or skeletal of the subject, for example, to treat a dystrophinopathy such as Duchenne muscular dystrophy (DMD), Becker muscular dystrophy, or DMD-associated dilated cardiomyopathy (DCM). Without wishing to be bound by theory, the AAV particles and methods for delivering the same to subjects in need thereof, to treat a dystrophinopathy, provide a superior benefit to known AAV particles and treatment methods at least because the present invention: (i) allows for significantly lower dosages than IV delivery, in order to achieve substantially the same or a better therapeutic benefit, thereby reducing viral load and toxicity and other side effects; and/or (ii) allows for preferential transgene targeting and expression in cardiac and/or skeletal muscle tissue compared to liver tissue, thereby targeting the transgene to cells of interest to provide a greater therapeutic benefit compared to AAV vectors delivered intravenously; (iii) can benefit greater patient populations compared to IV formulations, because of the lower dosages needed which corresponds to a decreased manufacturing burden.

In one embodiment, a method of treating a subject in need thereof is provided comprising intrathecally administering to the subject in a single dose, a composition comprising an effective amount of an AAV particle comprising an AAV capsid encapsidating a vector genome comprising a μDys transgene. In a further embodiment, the subject is positioned in the Trendelenburg position prior to the intrathecal administration. In one embodiment, intrathecal administration of the composition is in the absence of a non-ionic, low-osmolar contrast agent. In another embodiment, intrathecal administration is in the presence of a non-ionic, low-osmolar contrast agent.

The present invention is based in part on the finding that intrathecal administration of an effective amount of an AAV particle described herein allows for μDys transgene expression at higher levels in skeletal and/or cardiac muscle of the subject compared to transgene expression in liver tissue of the subject. For example, in one embodiment, subsequent to administration of the effective amount of the AAV particle, e.g., an AAV9 particle, the μDys transgene is expressed at higher levels in skeletal muscle of the subject, compared to the levels of μDys transgene expression in liver tissue. In another embodiment, subsequent to administration of the effective amount of the AAV particle, e.g., a recombinant AAV9 particle, the μDys transgene is expressed at higher levels in cardiac muscle of the subject, compared to the levels of μDys transgene expression in liver tissue. In yet another embodiment, subsequent to administration of the effective amount of the AAV particle, e.g., an AAV9 particle, the μDys transgene is expressed at higher levels in skeletal and cardiac muscle of the subject, compared to the levels of μDys transgene expression in liver tissue.

According to the embodiments described herein, transgene expression may refer to gene expression (i.e., by measuring mRNA levels) or expression of the corresponding protein. It will be understood by those of ordinary skill in the art that in order to determine levels of transgene expression in different tissue types, substantially the same amount of tissue, or substantially the same number of cells should be compared for gene expression levels. The higher levels of μDys transgene expression in the skeletal and/or cardiac muscle, in one embodiment, is at least about 10%, at least about 20%, at least about 30%, at least about 40%, at least about 50%, at least about 60%, at least about 70% or at least about 80% higher, compared to the amount of μDys transgene expression in the liver tissue. In a further embodiment, the μDys transgene comprises the nucleic acid sequence set forth in SEQ ID NO:5.

In one embodiment of the methods described herein, the method of delivering an effective dose of an AAV particle encapsidating a μDys transgene provides greater μDys transgene expression in skeletal and/or cardiac muscle, compared to a substantially identical dose of an AAV particle encapsidating a μDys transgene administered intravenously. In another embodiment, the method of delivering an effective dose of an AAV particle provides greater μDys transgene expression in skeletal and/or cardiac muscle, compared to when an effective amount of an AAV particle encapsidating a μDys transgene is administered intravenously. Transgene expression, in one embodiment, is measured about 1 week, about 1 month, about 2 months, about 3 months, about 4 months, about 5 months, about 6 months, about 7 months, about 8 months, about 9 months, about 10 months, about 11 months, about 12 months, about 18 months or about 24 months subsequent to administration of the composition comprising the effective amount of the AAV particle. The transgene, in one embodiment, comprises the nucleic acid sequence set forth in SEQ ID NO 5.

In another embodiment, an AAV particle encapsidating a μDys transgene of the present invention, when administered intrathecally, provides at least about 10%, at least about 20%, at least about 30%, at least about 40%, at least about 50%, at least about 60%, at least about 70% or at least about 80% greater transgene expression in the skeletal and/or cardiac muscle compared to the transgene expression in the same tissue type when the identical vector genome dose is administered intravenously. The identical vector genome need not contain the same regulatory elements and/or an identical transgene sequence or the same AAV capsid. However, the transgene administered intrathecally and intravenously encode for a μDys polypeptide.

In yet another embodiment, the ratio of [(skeletal and/or cardiac muscle μDys transgene expression)]/(liver μDys transgene expression)] measured subsequent to administration of the intrathecally administered AAV particle, is greater than the same ratio when the identical dose of an AAV particle encapsidating a μDys transgene, is administered intravenously. Transgene expression, in one embodiment, is measured about 1 week, about 1 month, about 2 months, about 3 months, about 4 months, about 5 months, about 6 months, about 7 months, about 8 months, about 9 months, about 10 months, about 11 months, about 12 months, about 18 months, about 24 months, about 36 months, about 48 months or about 60 months, subsequent to administration of the composition comprising the effective amount of the AAV particle.

In one embodiment, the effective amount of the AAV particle encapsidating a μDys transgene, when intrathecally administered, provides a greater efficacy or a greater therapeutic benefit, compared to an AAV particle encapsidating a μDys transgene administered intravenously, at the same dose.

In some embodiments, the intrathecal (IT) vector genome (vg) dose sufficient to provide a therapeutic response for one of the treatment methods described herein is about 90%, about 90% or less, about 85%, about 85% or less, about 80%, about 80% or less, about 75%, about 75% or less, about 70%, about 70% or less, about 60%, about 60% or less, about 50%, about 50% or less, about 40%, about 40% or less, about 30%, about 30% or less, about 25%, about 25% or less, about 10%, or about 10% or less, an intravenous (IV) vg dose of a vector genome encoding a μDys transgene sufficient to provide the same or substantially the same therapeutic response. The IT vector genome comprises a μDys transgene. The IT and IV μDys transgenes need not include the same nucleic acid sequence. In some embodiments, the IT vg dose sufficient to provide a therapeutic response for one of the treatment methods described herein is about 90% or less than the IV vg dose sufficient to provide the same or substantially the same therapeutic response. In some embodiments, the IT vg dose sufficient to provide a therapeutic response for one of the treatment methods described herein is about 75% or less than the IV vg dose sufficient to provide the same or substantially the same therapeutic response. In some embodiments, the IT vg dose sufficient to provide a therapeutic response for one of the treatment methods described herein is about 50% or less than the IV vg dose sufficient to provide the same or substantially the same therapeutic response. In some embodiments, the IT vg dose sufficient to provide a therapeutic response for one of the treatment methods described herein is about 25% or less than the IV vg dose sufficient to provide the same or substantially the same therapeutic response. In some embodiments, the IT vg dose sufficient to provide a therapeutic response for one of the treatment methods described herein is about 10% or less than the IV vg dose sufficient to provide the same or substantially the same therapeutic response. In some embodiments, the IT vg dose sufficient to provide a therapeutic response for one of the treatment methods described herein is about 10-40 times less than the IV vg dose sufficient to provide the same or substantially the same therapeutic response. The therapeutic response in one embodiment, is μDys transgene expression in muscle tissue, e.g., cardiac and/or skeletal muscle tissue. In another embodiment, the therapeutic response is an increase of the subject's score from baseline (i.e., prior to treatment) in the NorthStar Ambulatory Assessment (NSAA). In a further embodiment, the transgene encodes a μDys polypeptide.

In some embodiments, the intrathecal (IT) vector genome (vg) dose sufficient to provide a therapeutic response for one of the treatment methods described herein is lower than an intravenous (IV) vg dose sufficient to provide the same or substantially the same therapeutic response, wherein the IT and IV vector genomes each includes a transgene that encodes for a μDys polypeptide. However, the respective transgenes need not include the same nucleic acid sequence. In some embodiments, the IT vg dose sufficient to provide a therapeutic response for one of the treatment methods described herein is about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 150-fold, about 200-fold, about 250-fold, about 500-fold, or about 1000-fold lower than the IV vg dose sufficient to provide the same or substantially the same therapeutic response. In some embodiments, the IT vg dose sufficient to provide a therapeutic response for one of the treatment methods described herein is about 10-fold to about 20-fold, about 10-fold to about 30-fold, about 10-fold to about 40-fold, about 10-fold to about 50-fold, about 10-fold to about 75-fold, about 10-fold to about 100-fold, or about 10-fold to about 1000-fold lower than the IV vg dose sufficient to provide the same or substantially the same therapeutic response. In some embodiments, the IT vg dose sufficient to provide a therapeutic response for one of the treatment methods described herein is about 25-fold to about 30-fold, about 25-fold to about 40-fold, about 25-fold to about 50-fold, about 25-fold to about 75-fold, about 25-fold to about 100-fold, about 25-fold to about 500-fold, or about 25-fold to about 1000-fold lower than the IV vg dose sufficient to provide the same or substantially the same therapeutic response. In some embodiments, the IT vg dose sufficient to provide a therapeutic response for one of the treatment methods described herein is about 50-fold to about 75-fold, about 50-fold to about 100-fold, about 50-fold to about 250-fold, about 50-fold to about 500-fold, or about 50-fold to about 1000-fold lower than the IV vg dose sufficient to provide the same or substantially the same therapeutic response. In some embodiments, the IT vg dose sufficient to provide a therapeutic response for one of the treatment methods described herein is about 100-fold to about 200-fold, about 100-fold to about 250-fold, about 100-fold to about 500-fold, or about 100-fold to about 1000-fold lower than the IV vg dose sufficient to provide the same or substantially the same therapeutic response. In some embodiments, the IT vg dose sufficient to provide a therapeutic response for one of the treatment methods described herein is about 10-fold to about 40-fold lower than the IV vg dose sufficient to provide the same or substantially the same therapeutic response. In some embodiments, the IT vg dose sufficient to provide a therapeutic response for one of the treatment methods described herein is about 25-fold to about 40-fold lower than the IV vg dose sufficient to provide the same or substantially the same therapeutic response. In some embodiments, the IT vg dose sufficient to provide a therapeutic response for one of the treatment methods described herein is about 10-fold lower than the IV vg dose sufficient to provide the same or substantially the same therapeutic response. In some embodiments, the IT vg dose sufficient to provide a therapeutic response for one of the treatment methods described herein is about 25-fold lower than the IV vg dose sufficient to provide the same or substantially the same therapeutic response. In some embodiments, the IT vg dose sufficient to provide a therapeutic response for one of the treatment methods described herein is about 40-fold lower than the IV vg dose sufficient to provide the same or substantially the same therapeutic response. In some embodiments, the IT vg dose sufficient to provide a therapeutic response for one of the treatment methods described herein is about 50-fold lower than the IV vg dose sufficient to provide the same or substantially the same therapeutic response. In some embodiments, the IT vg dose sufficient to provide a therapeutic response for one of the treatment methods described herein is about 100-fold lower than the IV vg dose sufficient to provide the same or substantially the same therapeutic response. The therapeutic response in one embodiment, is transgene expression in muscle tissue, e.g., cardiac and/or skeletal muscle tissue.

In some embodiments, an effective dose of an intrathecal (IT) composition comprising the AAV particles encapsidating a μDys transgene described herein is lower than the effective dose of an intravenous (IV) composition comprising an AAV particle encapsidating a μDys transgene. In some embodiments, the effective dose of the IT composition comprising the AAV particle is about 90%, about 90% or less, about 85%, about 85% or less, about 80%, about 80% or less, about 75%, about 75% or less, about 70%, about 70% or less, about 60%, about 60% or less, about 50%, about 50% or less, about 40%, about 40% or less, about 30%, about 30% or less, about 25%, about 25% or less, about 10%, or about 10% or less vector genomes than the effective amount of the IV composition. In some embodiments, the effective amount of the IT composition comprising the AAV particle is about 90% or less vector genomes than the effective amount of the IV composition. In some embodiments, the effective amount of the IT composition comprising the AAV particle is about 75% or less vector genomes than the effective amount of the IV composition. In some embodiments, the effective amount of the IT composition comprising the AAV particle is about 50% or less vector genomes than the effective amount of the IV composition. In some embodiments, the effective amount of the IT composition comprising the AAV particle is about 25% or less vector genomes than the effective amount of the IV composition. In some embodiments, the effective amount of the IT composition comprising the AAV particle is about 10% or less vector genomes than the effective amount of the IV composition.

In some embodiments, an effective dose of an AAV particle in an IT composition is a lower dose than the effective dose of an AAV particle in an IV composition, wherein each AAV particle encapsidates a μDys transgene. In some embodiments, the effective dose of an AAV particle in an IT composition is about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 150-fold, about 200-fold, about 250-fold, about 500-fold, or about 1000-fold lower dose than the effective dose of the AAV particle in the IV composition. In some embodiments, the effective amount (effective dose) of the IT composition comprising the AAV particle is about 10-fold to about 20-fold, about 10-fold to about 30-fold, about 10-fold to about 40-fold, about 10-fold to about 50-fold, about 10-fold to about 75-fold, about 10-fold to about 100-fold, or about 10-fold to about 1000-fold lower dose than the effective amount (effective dose) of the IV composition comprising the same AAV particle, or an AAV particle encapsidating a transgene encoding the same polypeptide as the transgene encapsidated by the AAV particle in the IT composition. In some embodiments, the effective amount of the intrathecal composition comprising the AAV particle is about 25-fold to about 30-fold, about 25-fold to about 40-fold, about 25-fold to about 50-fold, about 25-fold to about 75-fold, about 25-fold to about 100-fold, about 25-fold to about 500-fold, or about 25-fold to about 1000-fold lower dose than the effective amount of the IV composition comprising the same AAV particle, or an AAV particle encapsidating a transgene encoding the same polypeptide as the transgene encapsidated by the AAV particle in the IT composition. In some embodiments, the effective amount of the intrathecal composition comprising the AAV particle is about 50-fold to about 75-fold, about 50-fold to about 100-fold, about 50-fold to about 250-fold, about 50-fold to about 500-fold, or about 50-fold to about 1000-fold lower dose than the effective amount of the IV composition comprising the same AAV particle, or an AAV particle encapsidating a transgene encoding the same polypeptide as the transgene encapsidated by the AAV particle in the IT composition. In some embodiments, the effective amount of the IT composition comprising the AAV particle is about 100-fold to about 200-fold, about 100-fold to about 250-fold, about 100-fold to about 500-fold, or about 100-fold to about 1000-fold lower dose than the effective amount of the IV composition comprising the same AAV particle. In some embodiments, the effective amount of the IT composition comprising the AAV particle is about 25-fold to about 40-fold lower than the effective amount of the IV composition comprising the same AAV particle, or an AAV particle encapsidating a transgene encoding the same polypeptide as the transgene encapsidated by the AAV particle in the IT composition. In some embodiments, the effective amount of the IT composition comprising the AAV particle is about 10-fold lower dose than the effective amount of the IV composition comprising the same AAV particle, or an AAV particle encapsidating a transgene encoding the same polypeptide as the transgene encapsidated by the AAV particle in the IT composition. In some embodiments, the effective amount of the IT composition comprising the AAV particle is about 25-fold lower dose than the effective amount of the IV composition comprising the same AAV particle, or an AAV particle encapsidating a transgene encoding the same polypeptide as the transgene encapsidated by the AAV particle in the IT composition. In some embodiments, the effective amount of the IT composition comprising the AAV particle is about 40-fold lower dose than the effective amount of the IV composition comprising the same AAV particle, or an AAV particle encapsidating a transgene encoding the same polypeptide as the transgene encapsidated by the AAV particle in the IT composition. In some embodiments, the effective amount of the IT composition comprising the AAV particle is about 50-fold lower dose than the effective amount of the IV composition comprising the same AAV particle, or an AAV particle encapsidating a transgene encoding the same polypeptide as the transgene encapsidated by the AAV particle in the IT composition. In some embodiments, the effective amount of the IT composition comprising the AAV particle is about 100-fold lower dose than the effective amount of the IV composition comprising the same AAV particle, or an AAV particle encapsidating a transgene encoding the same polypeptide as the transgene encapsidated by the AAV particle in the IT composition. In some embodiments, the transgene is a μDys transgene.

In one embodiment of the treatment methods provided herein, treating comprises decreasing in serum creatine kinase (CK) levels in a subject compared to the serum CK levels prior to treatment. In some embodiments, the serum CK levels are decreased by about 10%, about 20%, about 30%, about 40%, about 50%, about 60%, about 70%, about 80%, about 90%, or more compared to serum CK levels prior to treatment. In a further embodiment, serum CK levels are assessed prior to treatment with an AAV particle and at about 12 months, about 18 months, about 24 months, or about 30 months subsequent to administration of the AAV particle. In a further embodiment, the AAV particle comprises an AAV9 capsid encapsidating a μDys transgene.

In another embodiment of the intrathecal treatment methods provided herein, treating comprises decreasing the number of side effects, or a reducing the severity of one or more side effects in the subject being treated, compared to a subject treated via IV administration of an effective amount of the same AAV particle, or different AAV particle encapsidating a μDys transgene. The AAV particle administered intrathecally, in one embodiment, is an AAV9 particle.

In some embodiments, an AAV particle of the present invention or a pharmaceutical composition comprising the same can be used to treat a dystrophinopathy including, but not limited to, DMD, Becker muscular dystrophy, and DCM.

In some embodiments, the AAV particle of the present invention is administered once or multiple times to a subject in need of treatment, e.g., a subject with DMD. In some embodiments, the AAV particle is administered once, twice, three times, four times, five times, six times, seven times, eight times, nine times, ten times, or more to a subject in need of treatment. In a preferred embodiment, an intrathecal composition provided herein comprising the AAV particle is administered once to a subject in need of treatment. In a further embodiment, the AAV particle is an AAV9 particle.

In one embodiment, an effective amount of an AAV particle comprising an AAV capsid encapsidating a μDys transgene as described herein is used in a method for treating DMD in a subject in need of treatment. In a further embodiment, the subject is intrathecally administered a composition comprising the AAV particle in a single dose. In a further embodiment, the AAV particle is an AAV9 particle. In even a further embodiment, the subject is positioned in the Trendelenburg position prior to the administration.

In one embodiment of a method for treating DMD, a subject in need of treatment is intrathecally administered in a single dose, a composition comprising an effective amount of an AAV particle comprising an AAV capsid encapsidating a μDys transgene. The μDys transgene, in one embodiment, includes a combination of dystrophin elements set forth in FIG. 14. In one embodiment, the vector genome comprises a nucleic acid sequence set forth in SEQ ID NO:5, 10, 11 or 12.

In one embodiment of a method for treating DMD, treating comprises increasing the subject's score from baseline (i.e., prior to treatment) in the NorthStar Ambulatory Assessment (NSAA). The NSAA is a 17-item rating scale that is used to measure functional motor abilities in ambulatory DMD subjects. The scale is ordinal with 34 as the maximum score indicating fully independent function. Each activity is graded as either a 0 (unable to achieve independently), 1 (modified method but achieves goal independent of physical assistance from another), or 2 (normal—no obvious modification of activity). See, e.g., Mazonne et al. (2009). Neuromuscular Disorders 19, pp. 458-461 and researchrom.com/masterlist/view/18 #form2, the disclosure of each of which is incorporated by reference in its entirety for all purposes. The change from baseline, in one embodiment, is measured 12 months subsequent to administration of the intrathecal composition. In another embodiment, the change from baseline is measured 18 months subsequent to administration of the intrathecal composition. In another embodiment, the change from baseline is measure 24 months subsequent to administration of the intrathecal composition. In even another embodiment, the subject's increased score from baseline in the NSAA as measured 12 months subsequent to administration is substantially unchanged or increased at 18 months subsequent to administration. In yet even another embodiment, the subject's increased score from baseline in the NSAA as measured 12 months subsequent to administration is substantially unchanged or increased at 24 months subsequent to administration. In yet even another embodiment, the subject's increased score from baseline in the NSAA as measured 12 months subsequent to administration is substantially unchanged or increased at 60 months subsequent to administration.

Increasing the NSAA score, in one embodiment, comprises increasing the NSAA score by from about 5 to about 25, from about 5 to about 20, from about 5 to about 15 or from about 5 to about 10. In another embodiment, increasing the NSAA score, comprises increasing the NSAA score by from about 2 to about 12 points. In another embodiment, increasing the NSAA score, comprises increasing the NSAA score by from about 2 to about 10 points. In yet another embodiment, increasing the NSAA score, comprises increasing the NSAA score by from about 3 to about 10 points. In even another embodiment, increasing the NSAA score comprises increasing the score by from about 4 to about 10 points. In yet even another embodiment, increasing the NSAA score comprises increasing the score by from about 2 to about 8 points. I n another embodiment, increasing the NSAA score, comprises increasing the NSAA score by from about 2 to about 6 points.

In one embodiment of a method for treating DMD, a subject in need of treatment is intrathecally administered in a single dose, an effective amount of an AAV vector encapsidating a Dys transgene. The Dys transgene, in one embodiment, includes the combination of dystrophin elements set forth in FIG. 14. In one embodiment, the vector genome comprises the nucleic acid sequence set forth in SEQ ID NO:5, 10, 11 or 12. An effective amount of the AAV vector encapsidating a μDys transgene, in one embodiment, is an amount sufficient to increase the number of meters walked in a 6-minute walk test (6MWT), compared to the number of meters walked prior to treatment.

The AAV particles of the present invention, or pharmaceutical compositions comprising the same, are administered as a single dose or as divided doses. In some embodiments, the dose is 1×10⁹ to 1×10¹⁶ vector genomes (vg), 2.5×10¹³ to 1×10⁵ vg, 5×10¹³ to 1×10⁵ vg, 7.5×10¹³ to 1×10¹⁵ vg, 1×10¹⁴ to 1×10¹⁵ vg, 2.5×10¹⁴ to 1×10⁵ vg, 5×10¹⁴ to 1×10¹⁵ vg, 7.5×10¹⁴ to 1×10⁵ vg, 1×10¹³ to 7.5×10¹⁴ vg, 2.5×10¹³ to 7.5×10¹⁴ vg, 5×10¹³ to 7.5×10¹⁴ vg, 7.5×10¹³ to 7.5×10¹⁴ vg, 1×10¹³ to 5.0×10¹⁴ vg, 2.5×10¹³ to 5.0×10¹⁴ vg, 5×10¹³ to 5.0×10¹⁴ vg, 7.5×10¹³ to 5.0×10¹⁴ vg, 1×10¹³ to 2.5×10¹⁴ vg, 2.5×10¹³ to 2.5×10¹⁴ vg, 5×10¹³ to 2.5×10¹⁴ vg, 7.5×10¹³ to 2.5×10¹⁴ vg, 1×10¹³ to 1×10¹⁴ vg, 2.5×10¹³ to 1×10¹⁴ vg, 5×10¹³ to 1×10¹⁴ vg, 7.5×10¹³ to 1×10¹⁴ vg delivered as a single dose or as divided doses. For example, in some embodiments, AAV particles of the present invention or pharmaceutical compositions comprising the same, is administered as a single dose of 2.5×10¹³ vg. In another embodiment, AAV particles of the present invention or pharmaceutical compositions comprising the same, is administered as a single dose of 5×10¹³ vg. In yet another embodiment, AAV particles of the present invention or pharmaceutical compositions comprising the same, is administered as a single dose of 1×10¹⁴ vg.

The AAV particles of the present invention, or pharmaceutical compositions comprising the same, are administered as a single intrathecal dose. In some embodiments, doses for intrathecal delivery can be 1×10⁹ to 1×10¹⁶ vector genomes (vg), 1×10¹⁰ to 1×10¹⁶ vg 1×10¹ to 1×10¹⁶ vg, 1×10¹² to 1×10¹⁶ vg, 1×10¹³ to 1×10¹⁶ vg, 1×10¹⁴ to 1×10¹⁶ vg, 1×10¹⁵ to 1×10¹⁶ vg, 1×10⁹ to 1×10¹⁵ vg, 1×10⁹ to 1×10¹⁴ vg, 1×10⁹ to 1×10¹³ vg, 1×10⁹ to 1×10¹² vg, 1×10⁹ to 1×10¹¹ vg, 1×10⁹ to 1×10¹⁰ vg, 1×10¹³ to 1×10¹⁵ vector vg, 2.5×10¹³ to 1×10¹⁵ vg, 5×10¹³ to 1×10¹⁵ vg, 7.5×10¹³ to 1×10¹⁵ vg, 1×10¹⁴ to 1×10¹⁵ vg, 2.5×10¹⁴ to 1×10¹⁵ vg, 5×10¹⁴ to 1×10¹⁵ vg, 7.5×10¹⁴ to 1×10¹⁵ vg, 1×10¹³ to 7.5×10¹⁴ vg, 2.5×10¹³ to 7.5×10¹⁴ vg, 5×10¹³ to 7.5×10¹⁴ vg, 7.5×10¹³ to 7.5×10¹⁴ vg, 1×10¹³ to 5.0×10¹⁴ vg, 2.5×10¹³ to 5.0×10¹⁴ vg, 5×10¹³ to 5.0×10¹⁴ vg, 7.5×10¹³ to 5.0×10¹⁴ vg, 1×10¹³ to 2.5×10¹⁴ vg, 2.5×10¹³ to 2.5×10¹⁴ vg, 5×10¹³ to 2.5×10¹⁴ vg, 7.5×10¹³ to 2.5×10¹⁴ vg, 1×10¹³ to 1×10¹⁴ vg, 2.5×10¹³ to 1×10¹⁴ vg, 5×10¹³ to 1×10¹⁴ vg, 7.5×10¹³ to 1×10¹⁴ vg delivered as a single or as divided doses. For example, in some embodiments, AAV particles of the present invention or pharmaceutical compositions comprising the same, can be administered as a single intrathecal dose of 2.5×10¹³ vg. In another embodiment, AAV particles of the present invention or pharmaceutical compositions comprising the same, can be administered as a single intrathecal dose of 5×10¹³ vg. In yet another embodiment, AAV particles of the present invention or pharmaceutical compositions comprising the same, can be administered as a single intrathecal dose of 1×10¹⁴ vg.

In other embodiments, AAV particles of the present invention or pharmaceutical compositions comprising the same, can be administered as a single or divided intracerebroventricular doses. In some embodiments, doses for intracerebroventricular delivery can be 1×10¹³ to 1×10¹ vg, 2.5×10¹³ to 1×10¹⁵ vg, 5×10¹³ to 1×10¹⁵ vg, 7.5×10¹³ to 1×10¹⁵ vg, 1×10¹⁴ to 1×10¹⁵ vg, 2.5×10¹⁴ to 1×10¹⁵ vg, 5×10¹⁴ to 1×10¹⁵ vg, 7.5×10¹⁴ to 1×10⁵ vg, 1×10¹³ to 7.5×10¹⁴ vg, 2.5×10¹³ to 7.5×10¹⁴ vg, 5×10¹³ to 7.5×10¹⁴ vg, 7.5×10¹³ to 7.5×10¹⁴ vg, 1×10¹³ to 5.0×10¹⁴ vg, 2.5×10¹³ to 5.0×10¹⁴ vg, 5×10¹³ to 5.0×10¹⁴ vg, 7.5×10¹³ to 5.0×10¹⁴ vg, 1×10¹³ to 2.5×10¹⁴ vg, 2.5×10¹³ to 2.5×10¹⁴ vg, 5×10¹³ to 2.5×10¹⁴ vg, 7.5×10¹³ to 2.5×10¹⁴ vg, 1×10¹³ to 1×10¹⁴ vg, 2.5×10¹³ to 1×10¹⁴ vg, 5×10¹³ to 1×10¹⁴ vg, 7.5×10¹³ to 1×10¹⁴ vg. For example, in some embodiments, AAV particles of the present invention or pharmaceutical compositions comprising the same, can be administered as a single intracerebroventricular dose of 2.5×10¹³ vg. In another embodiment, AAV particles of the present invention or pharmaceutical compositions comprising the same, can be administered as a single intracerebroventricular dose of 5×10¹³ vg. In yet another embodiment, AAV particles of the present invention or pharmaceutical compositions comprising the same, can be administered as a single intracerebroventricular dose of 1×10¹⁴ vg.

### EXAMPLES

The invention now being generally described, will be more readily understood by reference to the following examples, which are included merely for purposes of illustration of certain aspects and embodiments of the present invention, and is not intended to limit the invention.

### Select Methods

### Intracerebroventricular (ICV) Injections at Postnatal Day 1 (p1)

Perform Intracerebroventricular injection (ICV) of neonatal pups (p1) via the Cerebral Hemisphere. Neonatals are injected fully awake. Ensure needles are marked for an injection depth of 2 mm. The injection site is the midpoint between the ear and eye (location is approximately 0.7-1.0 mm lateral to the sagittal suture and 0.7-1.0 mm caudal from the neonatal bregma). P0 is designated as the mouse Date of Birth (DoB). Animals may be injected within 36 hours of discovering the litter.

If an excessive amount of Dosing solution is determined to have leaked out of the injection site, or the injection site was missed entirely, the animal is excluded from the study.

Intracerebroventricular (ICV) Injections at Postnatal Day 28 (p28)

Hamilton syringe re loaded with desired volume of dosing solution. Standard volume is 8 μl per injection site. Animals are removed individually from cage and placed in anesthesia chamber. Once in chamber, the animal is anaesthetized. Tubing is connected from anesthesia machine to stereotaxic device to allow for continued anaesthetization during injection. The respective animal remains in chamber for about two minutes before being removed and placed on the stereotaxic device. Once the animal is on the stereotaxic device, coordinates for injection are set at medial/lateral (M/L): +/−1.00 mm, anterior/posterior (A/P): −0.5-−0.8 mm, dorsal/ventral (D/F): −2.5 mm.

An iodine swab is applied at incision site on scalp of animal for sterility purposes. Using a scalpel, a small incision is made into the scalp of the animal and the scalp is gently peeled back to expose cranial region. Once needle is in desired location, syringe plunger is slowly pushed down to inject dosing solution into cranial space. The site of injection is monitored during injection and immediately post injection to confirm quality of injection.

### Muscle Preparation

When animals reach the appropriate age, they are weighed and anesthetized (ketamine [80 mg/kg], acepromazine [0.5 mg/kg], and xylazine [16 mg/kg]) via intraperitoneal (i.p.) injection. Dissection of tissues is then carried out. Scissors are used to cut the skin at the ankle and then the skin on both lower legs is pulled back to expose the lower and upper leg muscles. The tibialis anterior on one side is dissected as close to its insertion point and weighed to the nearest 0.1 mg and then discarded. Then, 4.0 suture is tied at the myotendinous junction of the proximal and distal ends of the extensor digitorum longus (EDL) and then dissected and placed into Ringer's solution (137 mm NaCl, 5 mm KCl, 2 mm CaCl₂), 1 mm MgSO₄, 1 mm NaH₂PO₄, 24 mm NaHCO₃, 11 mm glucose containing 10 mg/liter curare) that is kept at room temperature.

After dissecting the EDL, the abdomen is opened, and blood is drawn using a 1 cc syringe from the inferior vena cava (about 300-500 μL). The blood sits at room temperature for 25-35 minutes and then is centrifuged at 3,500×g for 10 minutes at 4° C. After spinning the supernatant, serum is isolated and placed into a microcentrifuge tube and frozen at −80° C.

The Achilles tendon is then cut and pulled back to expose the soleus, plantaris and gastrocnemius. The soleus is dissected out and placed in a tube and frozen in liquid nitrogen-cooled isopentane. The plantaris is blunt dissected free of the gastrocnemius and then cut as proximally as possible and placed in a tube and frozen in liquid nitrogen-cooled isopentane and stored at −80° C.

The gastrocnemius is cut as proximally as possible, weighed to the nearest 0.1 mg and placed in a tube and frozen. The tibialis anterior from the contralateral side is dissected free, weighed to the nearest 0.1 mg and pinned on cork at resting length. The EDL from the same side will also be dissected free and pinned on the same cork at resting length. The cork is then immersed in liquid nitrogen-cooled isopentane. After about 30 to about 45 seconds, the cork is placed on dry ice, and wrapped in foil and stored at −80° C. The quadriceps muscle is dissected and placed in a tube and frozen in liquid nitrogen-cooled isopentane and stored at −80° C. A small piece of liver is dissected and placed in a tube and frozen in liquid nitrogen-cooled isopentane and stored at −80° C. The diaphragm is dissected, folded in half, and then folded again, and then placed on cork and pinned. Then, the cork is immersed in liquid nitrogen-cooled isopentane. After 30-45 seconds the cork is placed on dry ice, wrapped in foil, and stored at −80° C.

The whole heart is dissected, weighed to the nearest 0.1 mg and placed in a tube and frozen in liquid nitrogen-cooled isopentane. The tube is capped and placed in liquid nitrogen until storage at −80° C.

### Example 1: Generation of Recombinant Adeno-Associated Virus Particles

### Molecular Cloning of Micro-Dystrophin Gene Constructs

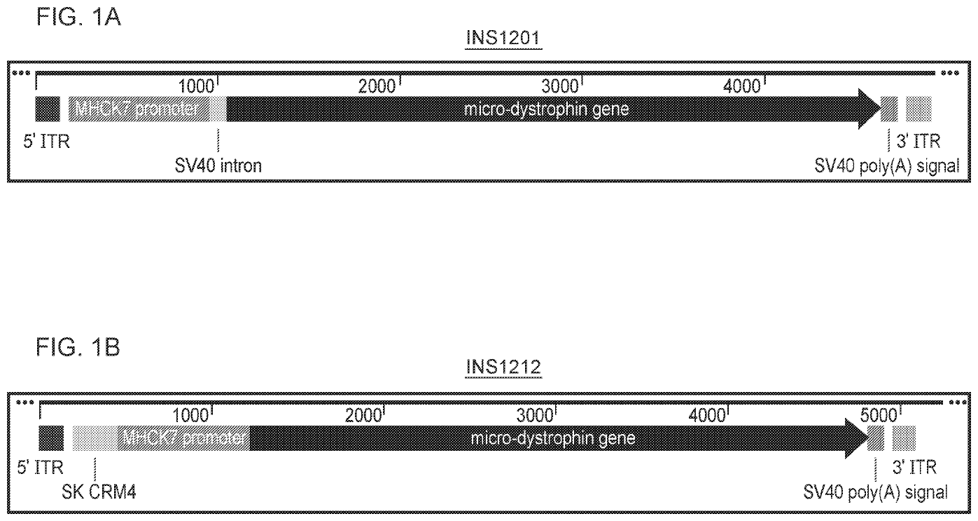

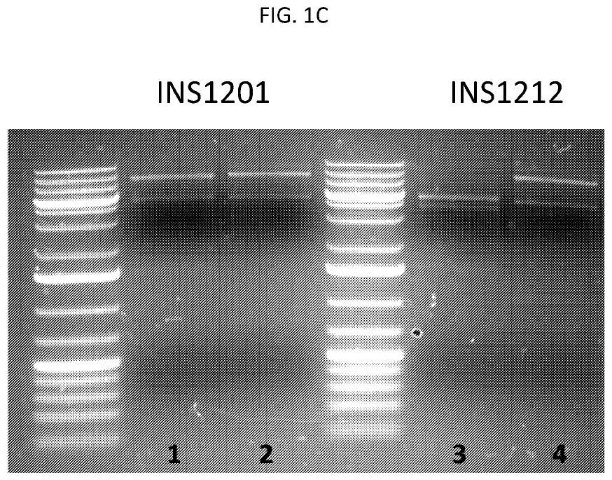

A micro-dystrophin (μDys) encoding gene construct, referred to herein as INS1201 and formerly known as MTS-001, was synthesized by operably linking an MHCK7 promoter and an SV40 intron to a polynucleotide encoding μDys and an SV40 poly(A) signal (FIG. 1A). An alternative μDys encoding gene construct (referred to herein as INS1212 and formerly known as MTS-003) was synthesized by operably linking an SK-CRM4 enhancer with an MHCK7 promoter to a polynucleotide encoding and an SV40 poly(A) signal (FIG. 1B). The INS1201 and INS1212 gene constructs were generated in a Puc57 vector backbone. The INS1201 and INS1212 constructs were confirmed by DNA sequencing and the 4542 bp and 4714 constructs, respectively, were isolated by NruI restriction digest and gel-purified for subsequent cloning into an appropriate AAV backbone.

The isolated INS1201 and INS1212 gene constructs were each independently blunt cloned into a gel-purified pSZ01 vector backbone containing ITR sites and a kanamycin resistance gene that was linearized/isolated by NruI restriction digest. Following T4 ligation of the INS1212 construct with the pSZ01 vector backbone and INS1212 construct with the pSZ01 vector backbone, DNA was transformed into E. coli, grown and purified using a NEB Monarch® Plasmid Miniprep kit. Gene constructs that had undergone successful ligation to produce complete pSZ01-INS1201 or psZ01-INS1212 plasmid clones were identified by HindIII/BsaI restriction digest.

pSZ01-INS1201 and psZ01-INS1212 clones were scaled-up by bacterial transformation in E. coli using a Maxi-Prep Kit (GeneJET Endo-free Plasmid Maxiprep Kit, ThermoScientific). Correct plasmid sequences were re-confirmed by BsaI/HindIII digest (FIG. 1C, lanes 1 and 3). Additionally, restriction digest using SmaI (FIG. 1C, lanes 2 and 4) was performed as further confirmation of correct incorporation of ITR sites containing INS1201 and INS1212 within the pSZ01 vector backbone.

### Transient Transfection and Viral Packaging

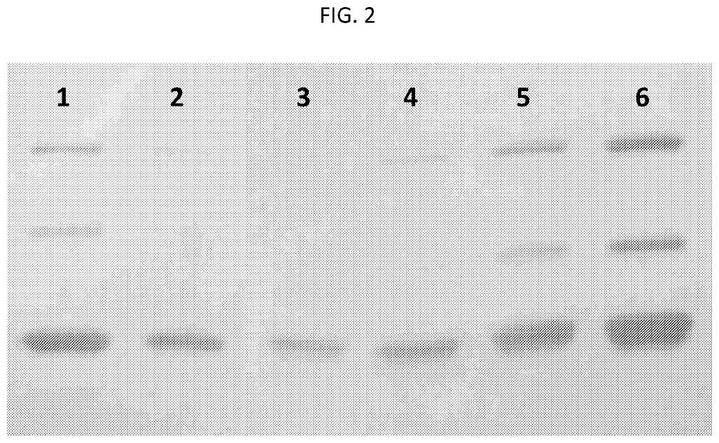

The confirmed pSZ01-INS1201 and psZ01-INS1212 AAV vectors were transiently transfected into HEK293 cells in combination with adenoviral helper plasmid and a chimeric packaging construct that delivers the AAV2 rep gene together with the AAV9 cap gene, using a standard calcium phosphate transfection method (for example, as described in Vandendriessche et al. (2007. J Thromb Haemost 5:16-24), incorporated by reference herein in its entirety). Two days post transfection, AAV particles were harvested and purified using two successive rounds of cesium chloride density gradient ultracentrifugation. 1 μl each of purified INS1201-AAV9 (FIG. 2, lane 1) and INS1212-AAV9 (FIG. 2, lane 2) were titered by comparing to 1×10¹³ vg AAV2 standard (FIG. 2, lane 3 (0.5p), lane 4 (1 μl), lane 5 (2 μl), lane 6 (4 μl)) resolved by SDS-PAGE and silver-stained.

### Example 2: Intramuscular Delivery of AAV9 μDys (INS1201-AAV9 and INS1212-AAV9) Results in Increased μDys Expression in MTX Mice·

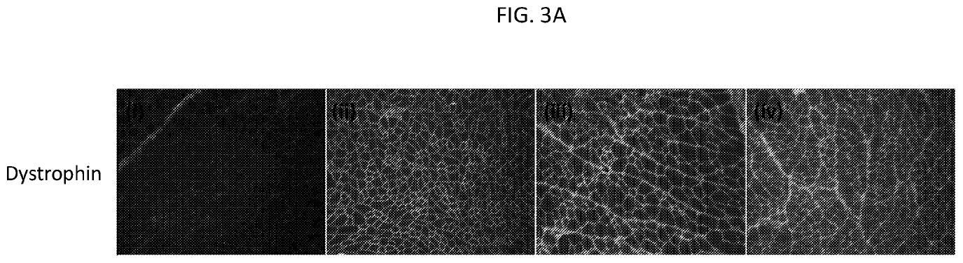

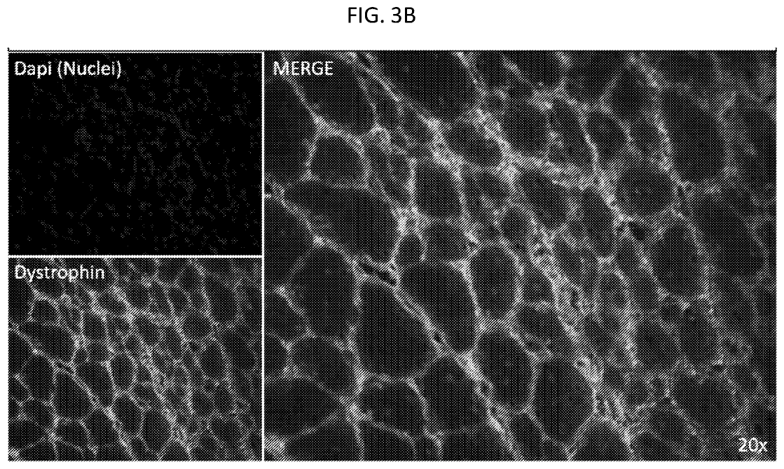

INS1201-AAV9 and INS1212-AAV9 were intramuscularly injected into the gastrocnemius muscle of mdx mice, a common murine model of Duchenne muscular dystrophy (see, for example Rodino-Klapac et al. (2013) Hum Mol Genet. 22(24):4929-37, incorporated herein by reference). As shown in FIG. 3A, 21 days-post intramuscular injection, gastrocnemius muscle injected with 2.7×10¹¹ vg of INS1201-AAV9 (iii) or INS1212-AAV9 (iv) exhibited widespread expression of μDys at significantly higher levels than non-injected mdx mice (i) and at comparable levels to wildtype C57/Bl mice (ii). FIG. 3B similarly shows high level expression of μDys in mdx mice 21 days post intramuscular injection with 2.7×10¹¹ vg of INS1212-AAV9.

### Example 3: Intracerebroventricular Delivery of AAV9 μDys (INS1201-AAV9 and INS 1212-AAV9) Results in Increased μDys Expression in MDX Mice

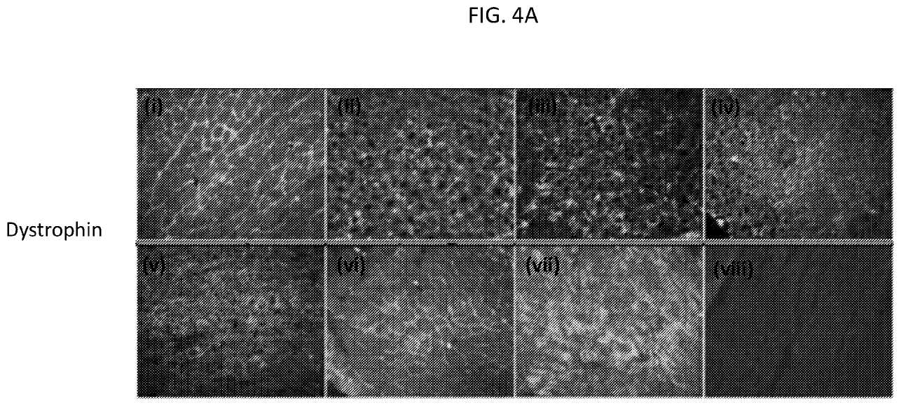

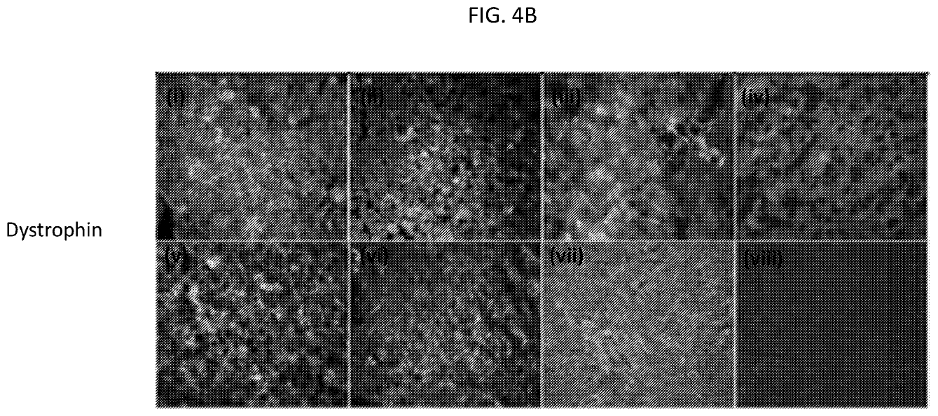

INS1201-AAV9 was intracerebroventicularly injected into mdx mice on postnatal day 1 (μl) and tissue samples were collected and analyzed for dystrophin. As shown in FIG. 4A, 21 days-post intracerebroventricular (ICV) injection with 1.8×10¹¹ vg of AAV, INS1201-AAV9 efficiently targeted and resulted in the expression of μDys in gastrocnemius (i), tibialis anterior (ii), quadriceps (iii), gluteus (iv), triceps (v), diaphragm (vi), and heart (vii) muscle cells with little to no expression in the liver (viii) of mdx animals. Similarly, as shown in FIG. 4B, 21 days-post ICV injection with 9×10¹⁰ vg of AAV, INS1201-AAV9 efficiently targeted and resulted in the expression of μDys in gastrocnemius (i), tibialis anterior (ii), quadriceps (iii), gluteus (iv), triceps (v), diaphragm (vi), and heart (vii) muscle cells with little to no expression in the liver (viii).

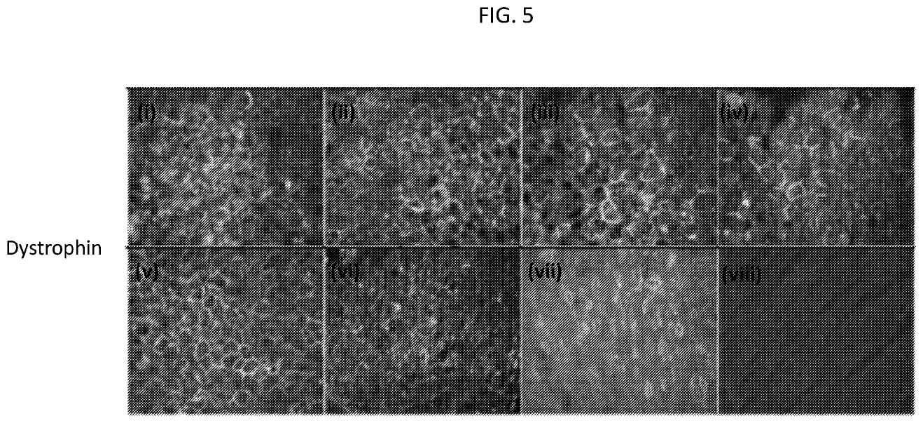

INS1212-AAV9 was also intracerebroventicularly injected into mdx mice on p1 and tissue samples were collected and immunofluorescently stained for dystrophin. As shown in FIG. 5, 21 days-post ICV injection with 9×10¹⁰ vg of AAV, INS1212-AAV9 efficiently targeted and resulted in the expression of μDys in gastrocnemius (i), tibialis anterior (ii), quadriceps (iii), gluteus (iv), triceps (v), diaphragm (vi), and heart (vii) muscle cells with little to no expression in the liver (viii).

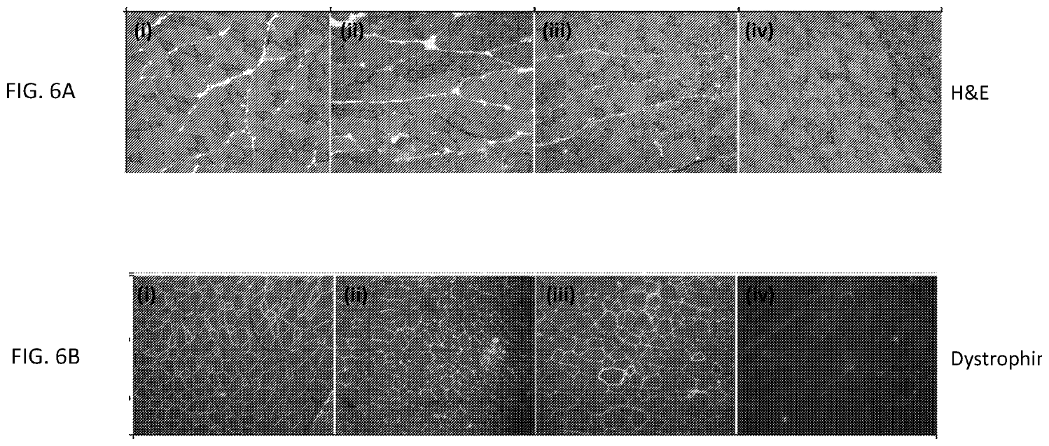

As shown in the hematoxylin and eosin-stained samples of FIG. 6A, 80 days post ICV injection with 9×10¹⁰ vg (ii) and 2.7×10¹¹ vg (iii) of INS1201-AAV9, gastrocnemius muscle tissue exhibited restoration of normal tissue architecture and correction of histopathological features of Duchenne muscular dystrophy as compared to wildtype C57/Bl (i) and non-injected mdx mouse controls (iv). As shown in FIG. 6B, at 80 days post ICV injection with 9×10¹⁰ vg (ii) and 2.7×10¹¹ vg (iii) of INS1201-AAV9, gastrocnemius muscle tissue exhibited levels of μDys comparable to dystrophin levels in wildtype C57/Bl mice (i), and significantly more than dystrophin levels in non-injected mdx mice (iv).

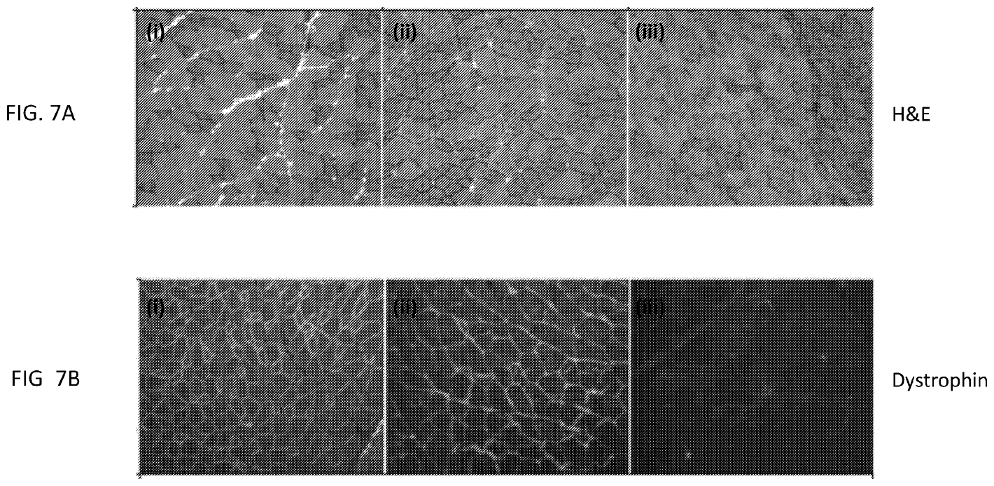

Similarly, as shown in the hematoxylin and eosin-stained samples of FIG. 7A(ii), 80 days post ICV injection with 9×10¹⁰ vg of INS1212-AAV9, gastrocnemius muscle tissue exhibited restoration of normal tissue architecture and correction of histopathological features of Duchenne muscular dystrophy as compared to wildtype C57/Bl (FIG. 7A(i)) and non-injected mdx mouse controls (FIG. 7A(iii)). As shown in FIG. 7B(ii), at 80 days post ICV injection with 9×10¹⁰ vg of INS1212-AAV9, gastrocnemius muscle tissue exhibited levels of μDys comparable to dystrophin levels in wildtype C57/Bl mice (FIG. 7B(i)), and significantly more than dystrophin levels in non-injected mdx mice (FIG. 7A(iii)).

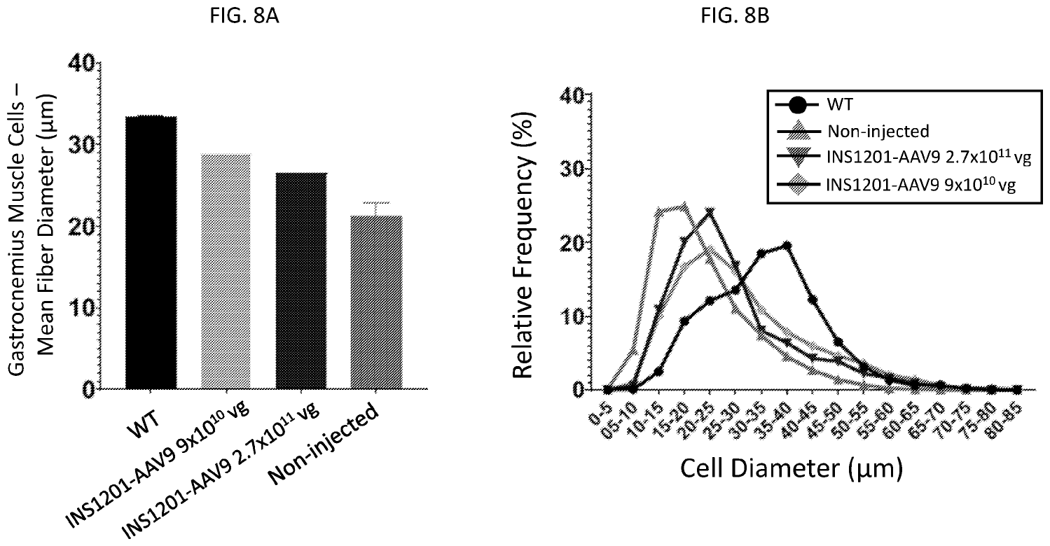

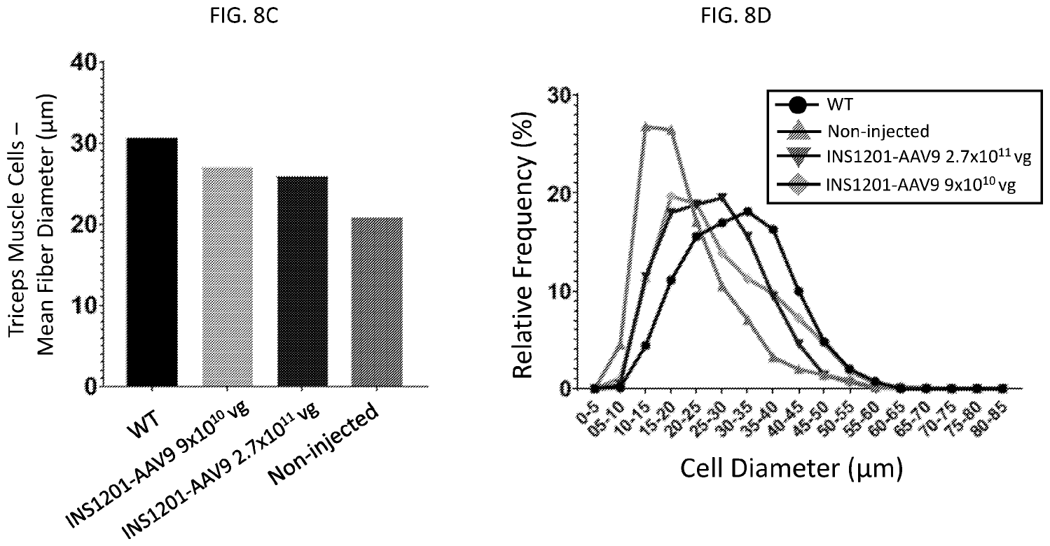

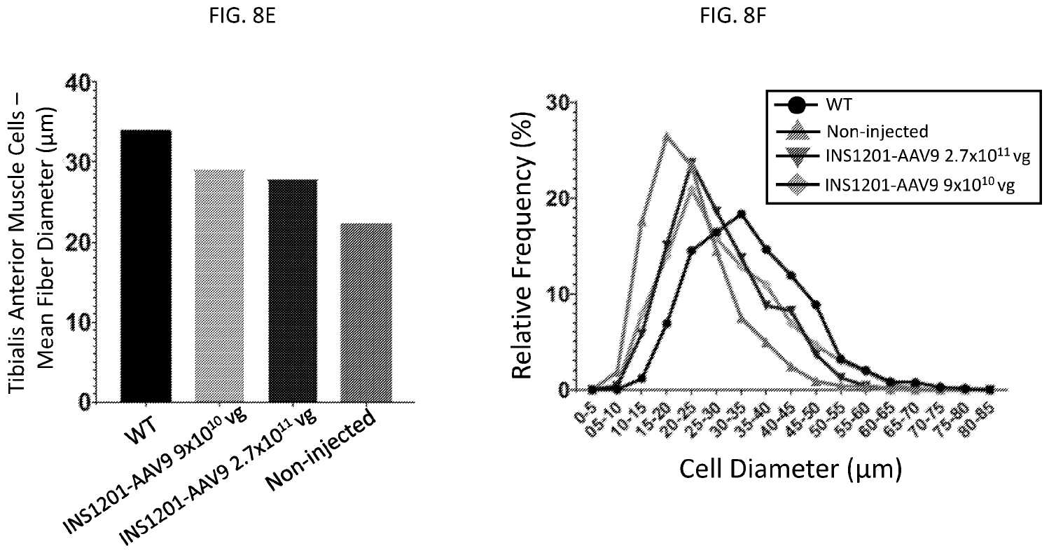

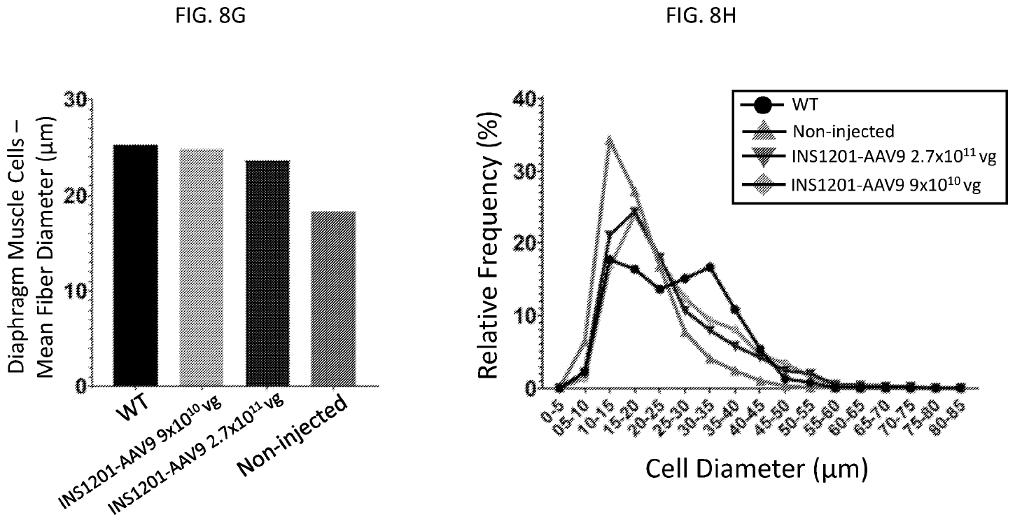

As shown in FIG. 8, gastrocnemius (FIG. 8A), triceps (FIG. 8C), tibialis anterior (FIG. 8E), and diaphragm (FIG. 8F) muscle tissue in mdx mice 80 days post ICV injection with 9×10¹⁰ vg or 2.7×10¹¹ vg of INS1201-AAV9 exhibited an increase in mean fiber diameter as compared to non-injected mdx mice. Mdx mice intracerebroventricularly injected with 9×10¹⁰ vg or 2.7×10¹¹ vg of INS1201-AAV9 also exhibited increased frequency of cells having larger diameters (e.g., 25 μm to 60 μm) 80 days post injection in gastrocnemius (FIG. 8B), triceps (FIG. 8D), tibialis anterior (FIG. 8F), and diaphragm (FIG. 8H), as compared to non-injected mice.

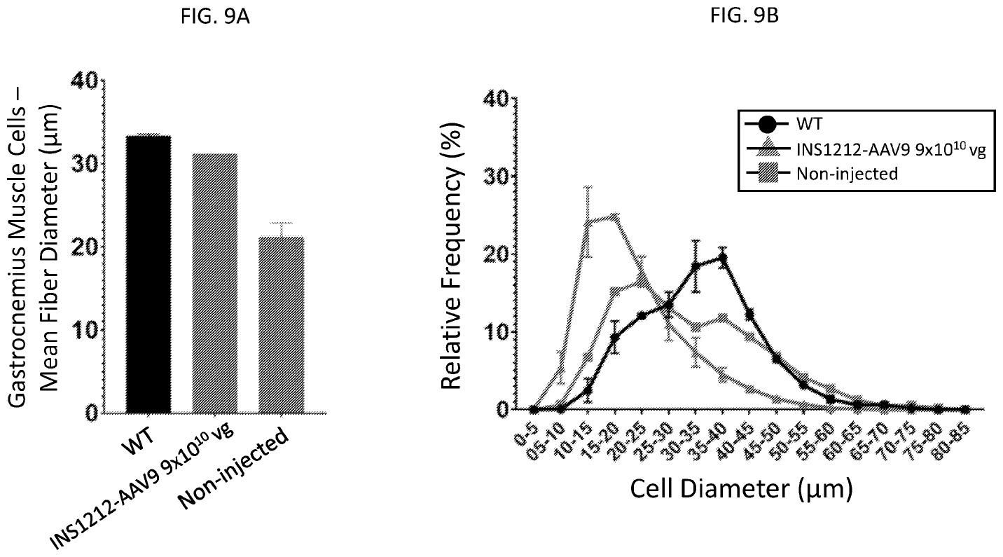

Similarly, as shown in FIG. 9A, gastrocnemius muscle tissue in mdx mice 80 days post ICV injection with 9×10¹⁰ vg of INS1212-AAV9 exhibited an increase in mean fiber diameter as compared to non-injected mdx mice with a concomitant increase in the frequency of cells having larger diameters (e.g., 25 μm to 60 μm) (FIG. 9B).

### Example 4: Intracerebroventricular Delivery of INS1201-AAV9 Results in Increased Dys Expression, Improved Muscle Histology and Reduced Fibrosis in Mdx Mice

Mdx mice at post-natal day 28 (p28) (with a window of −1 and +7 days such that no animal is less P27 and no animal greater than P35 at time of injection) were administered via intracerebroventricular (ICV) injection one of the following treatments: (i) 9×10⁹ vg of INS1201-AAV9 (n=6); (ii) 9×10¹⁰ vg of INS1201-AAV9 (n=7); (iii) 2.7×10¹¹ vg of INS1201-AAV9 (n=10); (iv) 5.4×10¹¹ vg of INS1201-AAV9 (n=7); (v) 1.2×10¹² vg of INS1201-AAV9 (n=8) or (vi) vehicle control (TFF formulation buffer, n=11). C57/BL1 age-matched mice were used as a wild type (WT) comparator (n=10).

Tissues were dissected as described above at approximately postnatal day 120.

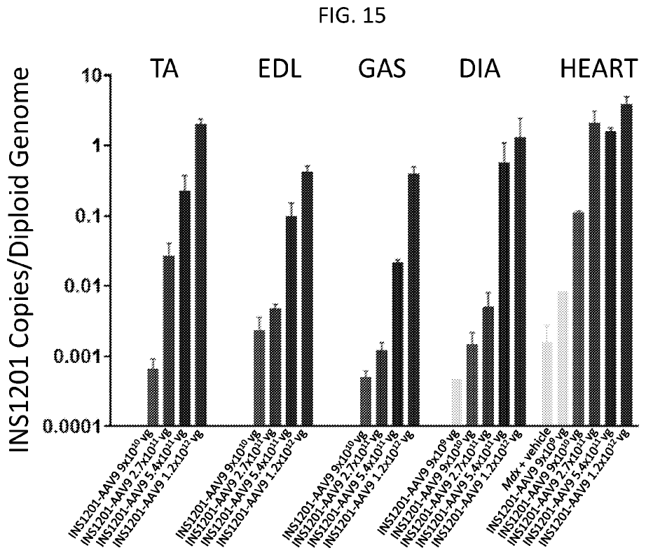

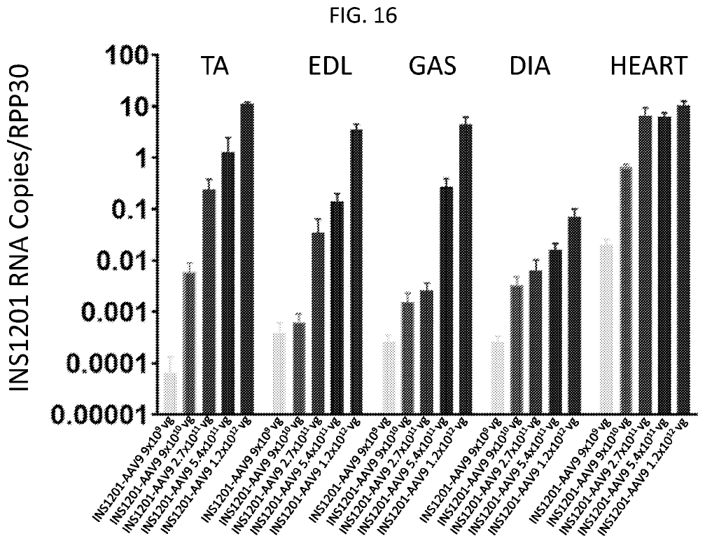

INS1201 copies were measured per diploid genome via droplet digital polymerase chain reaction (ddPCR) using primers specific for the INS1201 transgene. DNA copies of INS1201 are provided in FIG. 15. RNA transcript copies are provided in FIG. 16. TA: tibialis anterior; EDL: extensor digitorum longus; GAS: gastrocnemius; DIA: diaphragm. RPP30: ribonuclease P/MRP Subunit P30.

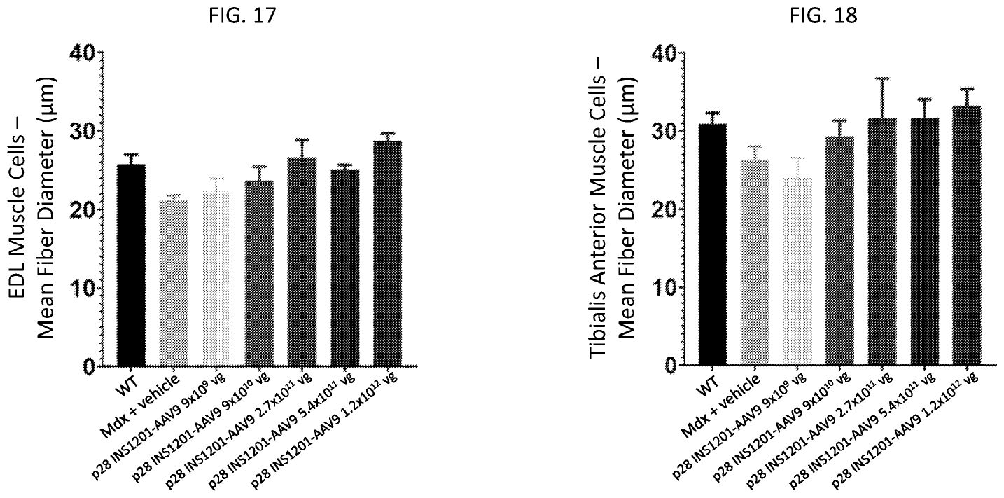

As shown in FIG. 17, mdx mice administered INS1201-AAV9 at all doses tested exhibited an increase in mean EDL fiber diameter, as compared to non-injected mdx mice. Consistent with these findings, mdx mice intracerebroventricularly injected with INS1201-AAV9 at all doses also exhibited increased frequency of cells having larger diameters (e.g., 25 μm to 60 μm) in EDL muscle, as compared to non-injected mice.

As shown in FIG. 18, mdx mice administered INS1201-AAV9 at the four highest doses tested exhibited an increase in mean TA fiber diameter, as compared to non-injected mdx mice. Consistent with these findings, mdx mice intracerebroventricularly injected with INS1201-AAV9 at the four highest doses tested (9×10¹⁰ vg; 2.7×10¹¹ vg; 5.4×10¹¹ vg; 1.2×10¹² vg) exhibited increased frequency of cells having larger diameters in TA muscle, as compared to non-injected mice.

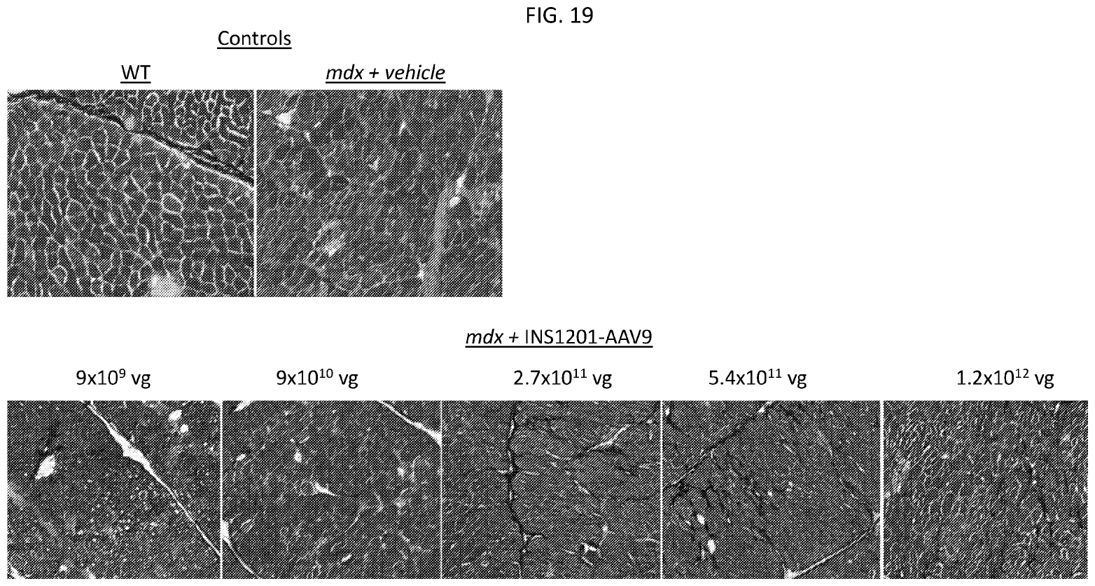

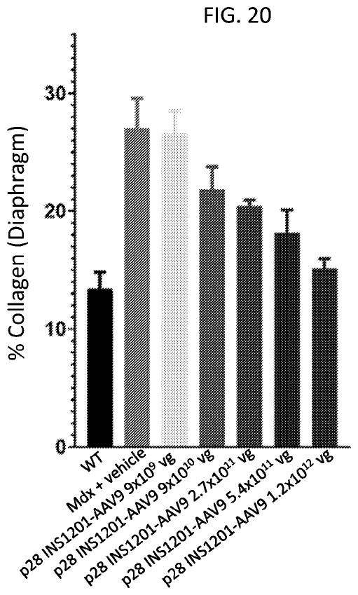

FIG. 19 shows diaphragm muscle sections stained with picrosirius red, post intracerebroventricular (ICV) injection with various doses of INS1201-AAV9. Picrosirius red is used to visualize collagen content. Diaphragm sections obtained from a wildtype C57/Bl mouse and mdx mouse administered vehicle are shown for comparison (top panels). As shown in the lower panels of FIG. 19, the mdx mice administered INS1201-AAV9 at the five doses tested (9×10⁹ vg; 9×10¹⁰ vg; 2.7×10¹¹ vg; 5.4×10¹¹ vg; 1.2×10¹² vg) exhibited decreased fibrosis, compared to a mdx mouse administered vehicle, as measured by collagen content. Percent collagen in diaphragm muscle is also provided in FIG. 20.

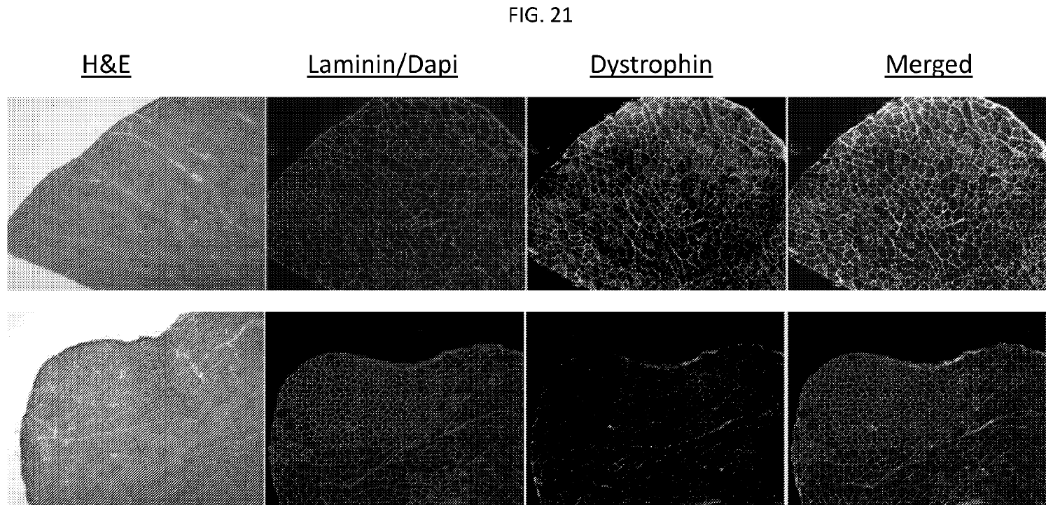

FIG. 21, top, are microphraphs of EDL sections obtained from an mdx mouse at postnatal day 120 (p120) subsequent to intracerebroventricular (ICV) injection at p28 with 5.4×10¹¹ vg of INS1201-AAV9 and stained with hematoxylin and eosin (H&E) (far left); laminin/dapi (second from left); dystrophin (second from right); and a merged image (far right). FIG. 21, bottom, shows a EDL muscle section obtained from an mdx mouse at postnatal day 120 (p120) subsequent to intracerebroventricular (ICV) injection at p28 with vehicle control, and stained with hematoxylin and eosin (H&E) (far left); laminin/dapi (second from left); dystrophin (second from right); and a merged image (far right). The staining shows greater dystrophin expression in the sample obtained from the mouse treated with INS1201-AAV9 compared to the sample taken from the control mouse. Muscle from the INS1201-AAV9 treated mouse also appear healthier, as evident from the H&E and laminin/dapi stains.

### Example 5: ICV Delivery of INS-1201 AAV9 Results in Improved Muscle Physiology in Mdx Mice

Mdx mice at post-natal day 1 (μl) were administered via intracerebroventricular (ICV) injection one of the following treatments: (i) 9×10⁹ vg of INS1201-AAV9; (ii) 9×10¹⁰ vg of INS-1201-AAV9; (iii) 2.7×10¹¹ vg of INS1201-AAV9; (iv) vehicle control (TFF formulation buffer). C57/BL10 age-matched mice (WT) were used as a comparator.

Mdx mice at post-natal day 28 (p28) (with a window of −1 and +7 days such that no animal is less P27 and no animal greater than P35 at time of injection) were administered via intracerebroventricular (ICV) injection one of the following treatments: (i) 9×10⁹ vg of INS1201-AAV9; (ii) 9×10¹⁰ vg of INS1201-AAV9; (iii) 2.7×10¹¹ vg of INS1201-AAV9; (iv) 5.4×10¹¹ vg of INS1201-AAV9; (v) 1.2×10¹² vg of INS1201-AAV9 or (vi) vehicle control (TFF formulation buffer). C57/BL1 age-matched mice were used as a wild type (WT) comparator.

Muscle dissection and preparation was carried out as described above when animals were from 120 to 135 days old.

### Muscle Physiology Experimental Design

### Muscle Mechanics in EDL

The EDL is mounted in a specialized chamber containing Ringer's solution (137 mM NaCl, 5 mM KCl, 2 mM CaCl₂), 1 mM MgSO₄, 1 mM NaH₂PO₄, 24 mM NaHCO₃, 11 mM glucose containing 10 mg/liter curare) at room temperature. The muscle origin is tied to a rigid post, and the insertion is secured to the arm of a dual-mode ergometer (model 300B; Aurora Scientific, ON, Canada), which allows precise control of muscle length.

Muscle activation is provided via an electrical stimulator using parallel platinum plate electrodes that extend the length of the muscle. Supramaximal stimulation conditions are established for each experiment by single 0.3 ms twitch pulses of increasing voltage, using +50% more than the value where the force plateaued for experimental testing. Optimal muscle length for assessing force characteristics is determined using a series of twitches while incrementally increasing muscle length (10% increments from slack length) and are defined as the length at which supramaximal stimulation produces maximal twitch force. After establishing optimal length, muscle fiber length (Lf) is measured and the muscle is rested for 2 min.

The muscle is then stimulated with two twitches spaced 60 s apart (0.3 ms pulse duration) to assess twitch characteristics (twitch tension, half-relaxation time, and time-to-peak tension).

The muscle is stimulated at increasing frequencies (400 ms train duration and 0.3 ms pulse duration; (i) EDL: 1 Hz, 10 Hz, 30 Hz, 50 Hz, 70 Hz, 120 Hz; (ii) soleus: 1 Hz, 5 Hz, 10 Hz, 20 Hz, 40 Hz, 60 Hz, 80 Hz, and 100 Hz) with 120 s intervals between contractions to determine the force-frequency (F-F) relationship.

Following the F-F testing, optimal length is re-verified by empirically testing maximal twitch force up to +20% of the length at the end of testing.

### Eccentric Contractions

After completing the F-F curve, all stretches or contractions are performed with two-minute intervals, and stimulation is performed at 100 Hz with 400 ms train duration and 0.3 ms pulse duration for isometric and eccentric contractions. Specifically, following a two-minute rest period from the end of the F-F assessment, the passive mechanical properties of the muscle are measured by imposing a 10% Lf stretch at 0.7 Lf/s twice with a 500 ms hold before returning to the starting muscle length. Then, two isometric contractions are obtained as a measurement of pre-eccentric contraction (EC) maximal isometric force.

Each muscle then undergoes an EC bout of ten contractions, during which the muscle is stimulated isometrically for the first 200 ms, after which a 15% Lf length change is imposed at a rate of 2 Lf/s. The muscle is then held at this length for 500 ms before returning to the starting muscle length. After 10 EC contractions, two isometric contractions are obtained to determine the post-EC maximal isometric force. At the completion of the final isometric measurements, two additional passive stretches are obtained to determine post-EC passive mechanical measurements.

Following contraction testing, muscles are trimmed of external tendons, blotted dry and weighed to the nearest 0.01 mg. Physiological cross-sectional areas of the muscles are calculated as function of fiber length and muscle mass. For all analysis, force is normalized to the muscle physiological cross-sectional area and reported as stress.

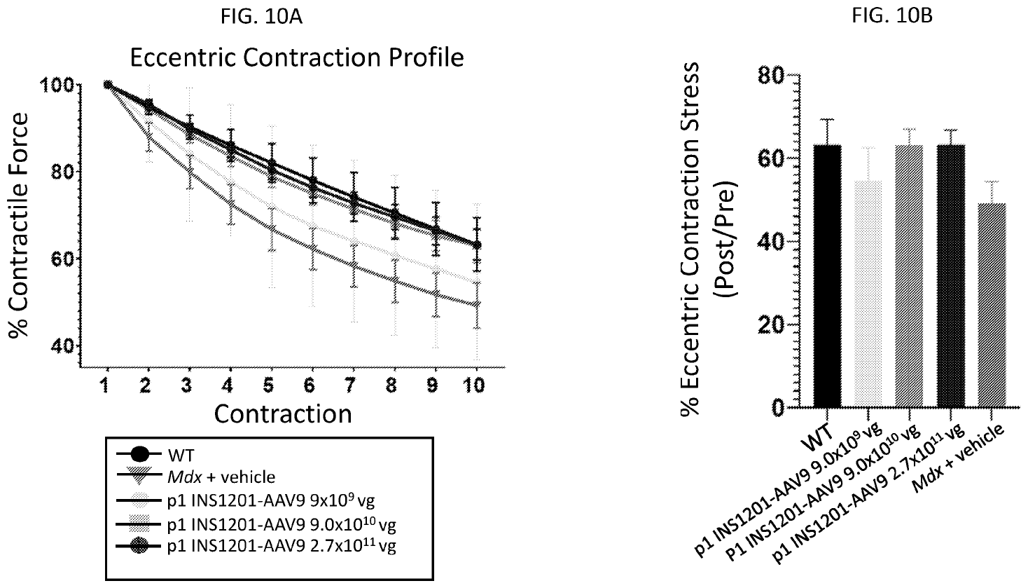

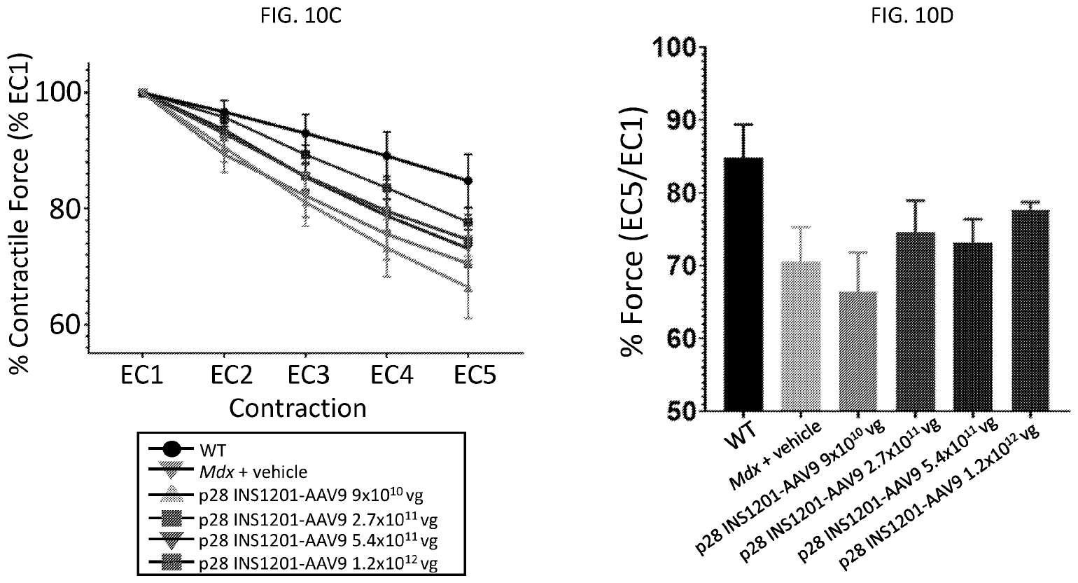

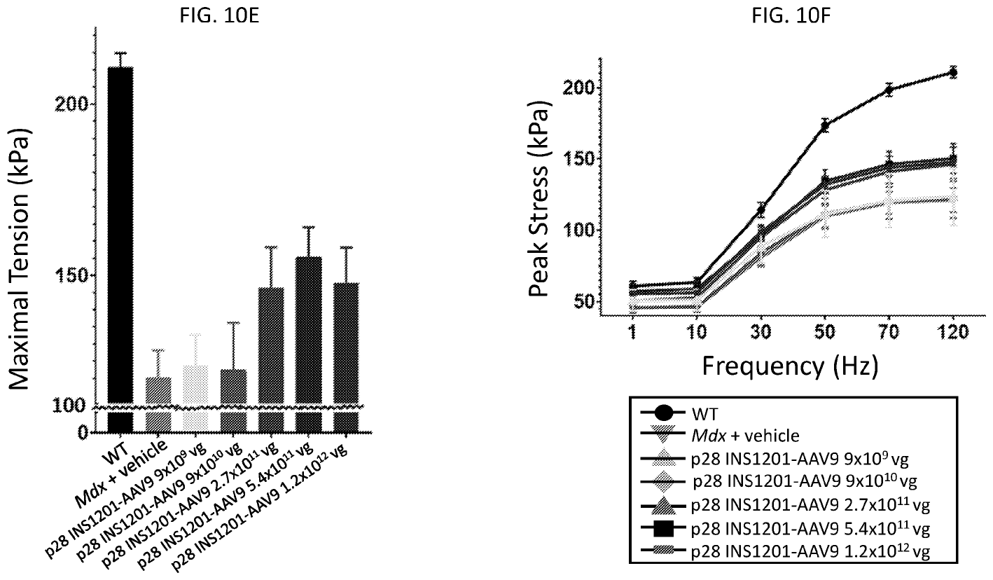

Mdx mice receiving ICV injection on postnatal day 1 (p1) of INS1201-AAV9 at all doses tested (9×10⁹ vg; 9×10¹⁰ vg; 2.7×10¹¹ vg) exhibited an attenuated decrease in percent contractile force resulting from EC as compared to mdx mice treated with vehicle (FIG. 10A). FIG. 10B similarly shows that mice receiving ICV injection of INS1201-AAV9 on p1, at the three doses tested (9×10⁹ vg; 9×10¹⁰ vg; 2.7×10¹¹ vg), exhibited improved muscle physiology as indicated by increased percent post-eccentric contraction stress relative to pre-eccentric contraction stress as compared to vehicle treated mice.

Mdx mice receiving ICV injection on postnatal day 28 (p28) of INS1201-AAV9 at three highest doses tested (2.7×10¹¹ vg; 5.4×10¹¹ vg; 1.2×10¹² vg) exhibited an attenuated decrease in percent contractile force resulting from EC as compared to mdx mice treated with vehicle (FIG. 10C, 10D).

Mdx mice receiving ICV injection on postnatal day 28 (p28) of INS1201-AAV9 at three highest doses tested (2.7×10¹¹ vg; 5.4×10¹¹ vg; 1.2×10¹² vg) exhibited improved and stabilized muscle function as shown by an increase in peak muscle force following stimulation at various frequencies as compared to mdx mice treated with vehicle (FIG. 10E, FIG. 10F).

### Example 6: One-Dose Intrathecal Administration of AAV9 Targets Transgene Delivery to Skeletal and Cardiac Muscle Tissue

AAV9-CBA-GFP was intrathecally administered to cynomolgus macaques who were screened for anti-AAV9 antibodies using a highly specific anti-AAV9 ELISA. Cynomolgus subjects were treated with 2.5×10¹³ vg, 5×10¹³ vg, or 1×10¹⁴ vg of AAV9-CBA-GFP via a single intrathecal dose and GFP expression was determined by immunohistochemical analysis with NovaRed GFP immunostain and Vector® HRP substrate, RT-PCR, and western blot analysis.

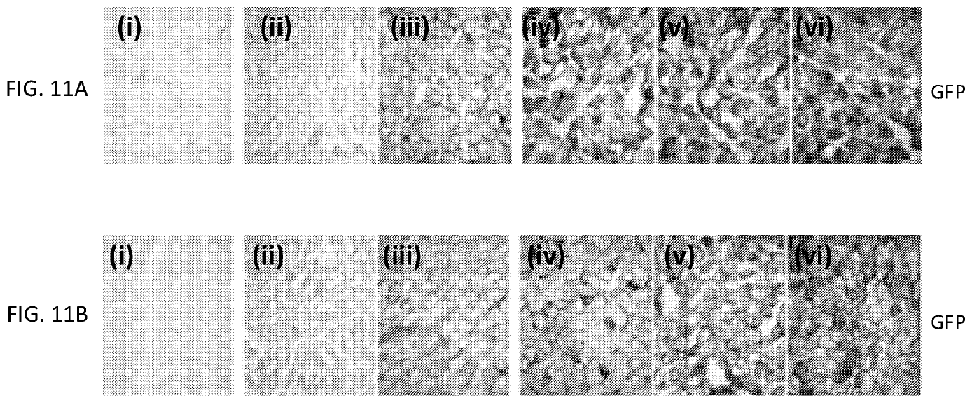

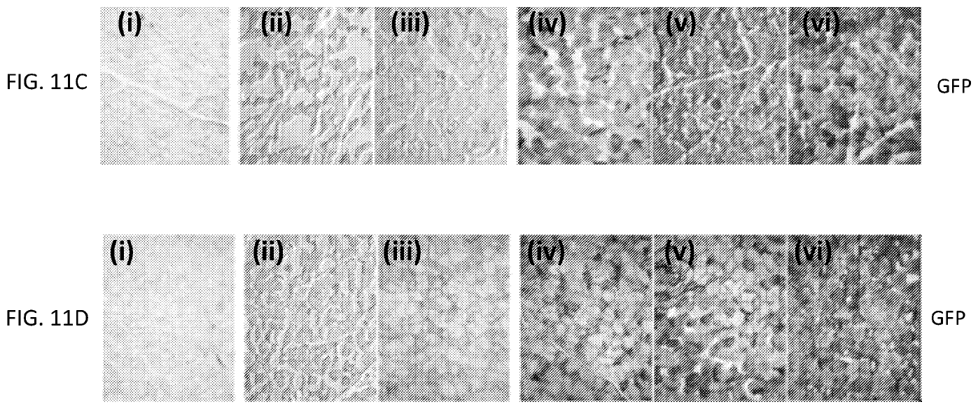

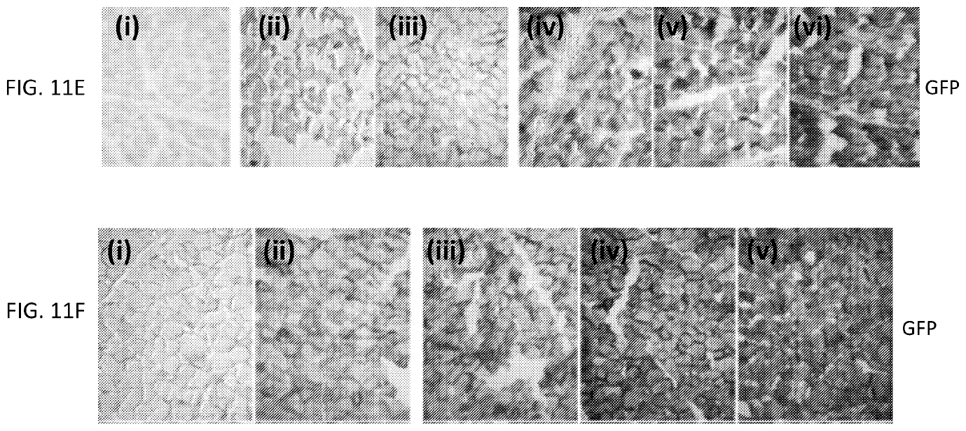

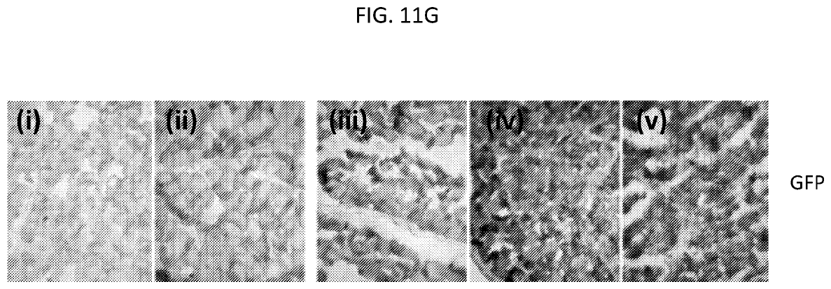

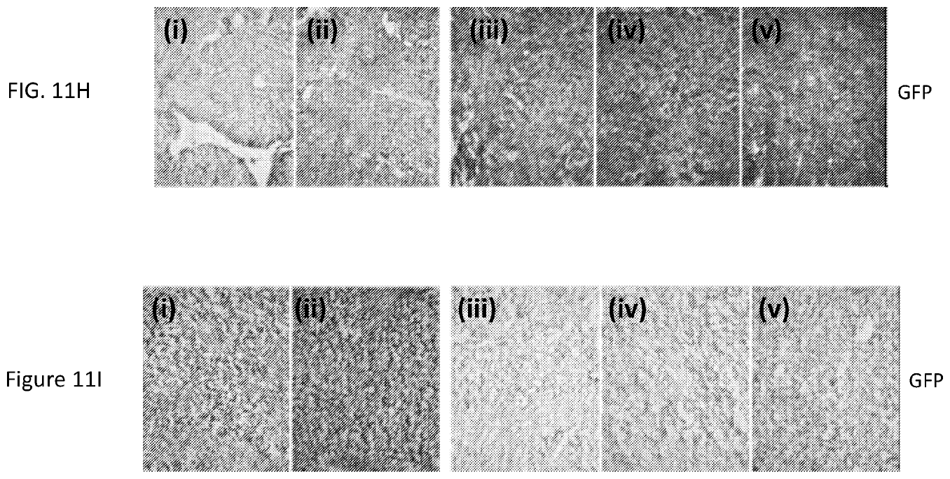

As shown in FIG. 11, cynomolgus macaques receiving a single intrathecal dose of AAV9-CBA-GFP at 2.5×10¹³ vg (iv), 5×10¹³ vg (v), or 1×10¹⁴ vg (vi), exhibited increased staining for GFP in gastrocnemius (FIG. 11A), quadriceps (FIG. 11B), deltoid (FIG. 11C), triceps (FIG. 11D), and biceps (FIG. 11E) muscle tissue as compared to cynomolgus subjects receiving 5×10¹³ vg (ii), or 1×10¹⁴ vg (iii) AAV9-CBA-GFP administered intravenously. Similarly, cynomolgus macaques receiving a single intrathecal dose of AAV9-CBA-GFP at 2.5×10¹³ vg (iii), 5×10¹³ vg (iv), or 1×10¹⁴ vg (v), exhibited increased staining for GFP in diaphragm (FIG. 11F), tibialis anterior (FIG. 11G), and heart (FIG. 11H) muscle tissue as compared to cynomolgus subjects receiving 5×10¹³ vg (i), or 1×10¹⁴ vg (ii) AAV9-CBA-GFP administered intravenously. As shown in FIGS. 11A-E (i), non-injected cynomolgus subjects exhibited little to no GFP staining.

As shown in FIG. 11I, cynomolgus macaques receiving a single intravenous dose of AAV9-CBA-GFP at 5×10¹³ vg (i), or 1×10¹⁴ vg (ii), exhibited high levels of GFP staining in liver tissue, whereas subjects receiving a single intrathecal dose of AAV9-CBA-GFP at 2.5×10¹³ vg (iii), 5×10¹³ vg (iv), or 1×10¹⁴ vg (v), exhibited significantly lower GFP staining.

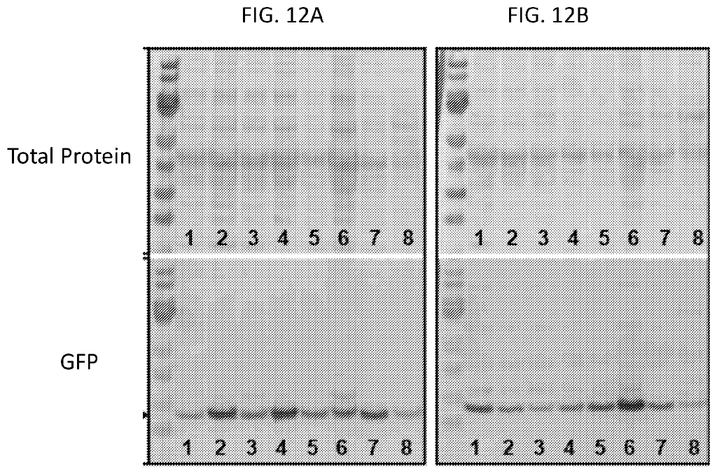

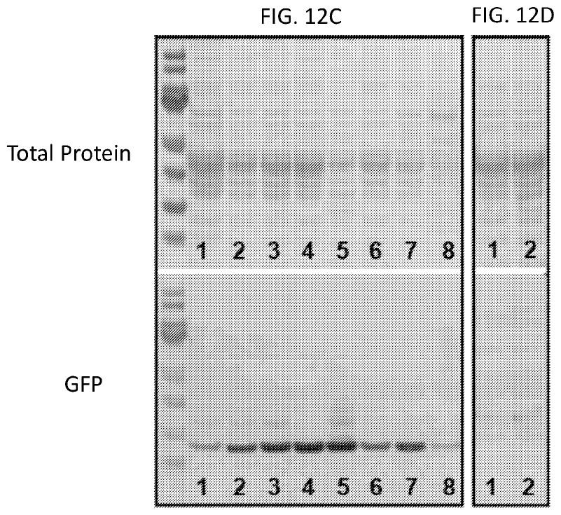

As shown in FIG. 12, immunohistochemical staining for GFP shown in FIG. 11 corresponded to protein levels as detected by anti-GFP western blot. Cynomolgus macaques receiving a single intrathecal dose of AAV9-CBA-GFP at 2.5×10¹³ vg (FIG. 12A), 5×10¹³ vg (FIG. 12B), or 1×10¹⁴ vg (FIG. 12C), exhibited increased GFP levels in biceps (1), triceps (2), deltoid (3), quadriceps (4), gastrocnemius (5), tibialis anterior (6), diaphragm (7) and heart (8) muscle tissue, whereas no GFP protein was detected by western blot in the biceps (FIG. 12D, 1) or triceps (FIG. 12D, 2) of non-injected subjects.

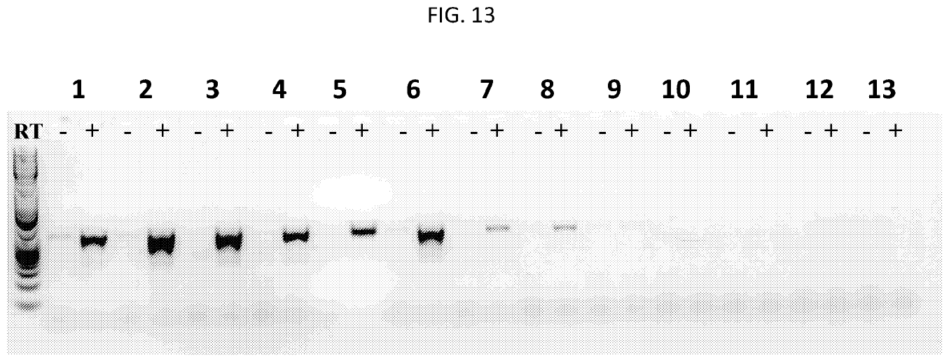

As shown in FIG. 13, GFP protein levels shown in FIGS. 11 and 12 corresponded to GFP mRNA levels, wherein cynomolgus macaques receiving a single intrathecal dose of AAV9-CBA-GFP exhibited detectable GFP mRNA expression in biceps (1), triceps (2), deltoid (3), tibialis anterior (4), gastrocnemius (5), quadriceps vastus lateralis (6), diaphragm (7), and heart (8) muscle tissue as analyzed by RT-PCR. Minimal GFP mRNA was detected in liver tissue (9) and no GFP mRNA was detectable in biceps (10), triceps (11), deltoid (12) and quadriceps (13) muscle tissue collected from non-injected subjects.

### Example 7: Toxicity and Biodistribution Study of INS1201-AAV9 Following Single Dose Administration of Intracerebroventricular Injection in Juvenile C57BL/6J Mice

### Test System

Species: Mus musculus
Strain: C57BL/6J
Sex: Male
Age: Approximately 4 weeks on Day 1
Weight: Commensurate with age
Number: 192 (+60 extra)
Caging: As per ASC SOPs
Minimum Acclimation: 5 days

Species/Strain, Number and Sex. Up to 252 male C57BL/6J (strain #000664) mice, approximately 4 weeks old at study start, are acquired for this study. Only male mice are used in the study, as the intended patient population is male only.

Starting Age and Weight Range. Animals selected for use in this study will be as uniform in age and weight as possible at start of study. Animals will be approximately 3 weeks of age upon delivery and approximately 4 weeks old on day 1 of study.

Animals will be fed a species-specific diet, fed ad libitum. No contaminants are known to be present in the diet at levels that would interfere with the results of this study. Irradiated water will be available ad libitum to each animal.

The test article and control articles used in this study are provided in Table 2 and Table 3, respectively.

TABLE 2
Test Articles.

| | | Active | |
|---|---|---|---|
| Article | Dose | Ingredient | Concentration |
| INS1201-AAV9 | 1 (Group 2) | AAV-micro- | 5 × 10¹³ vg/mL |
| | 8.0 × 10¹¹ vg | dystrophin | |
| INS1201-AAV9 | 2 (Group 3) | AAV-micro- | 5 × 10¹³ vg/mL |
| | 4.0 × 10¹¹ vg | dystrophin | |
| INS1201-AAV9 | 3 (Group 4) | AAV-micro- | 2.5 × 10¹³ vg/mL |
| | 2.0 × 10¹¹ vg | dystrophin | |

TABLE 3
Control Article.

| | | Active | |
|---|---|---|---|
| Vehicle Control | Dose | Ingredient | Concentration |
| Formulation | Vehicle | Pluronic F-68 | 0.006% |
| Buffer | (Group 1) | | |

### Experimental Design

### Study Design

The study consists of three cohorts: animals assigned to cohort 1 will be terminated on Day 85±10 following injection procedure, animals assigned to cohort 2 will be terminated on Day 43±7 following injection procedure and cohort 3 will be terminated on Day 22±3 following injection procedure. The overall study design is presented in Table 4. Extra animals may be dosed and be used to replace any unscheduled deaths of study group animals that occur as a direct result of procedural activity (i.e., injection, restraint, and handling during injection and/or blood collection). Animals that do not survive the test article administration procedure, die or require early termination prior to Day 4 (3 days post-injection) may be replaced. These animals will not be subject to a gross necropsy. Animals that do not meet the inclusion criteria for age may be replaced as necessary. All animal deaths will be reported, regardless of timing of death postinjection. For Groups 1-4 at each given time point, 5 animals will be used for hematology, blood ddPCR and tissue ddPCR; 5 animals will be used for clinical chemistry and histopathology; and 5 animals for coagulation test and histopathology (n=15/group/time point). All Group 5 animals will be euthanized in Week 12 for hematology, clinical chemistry and coagulation (n=4 for each test). Five (5) animals from Group 5 will be used for histopathology while tissues from the rest of the animals will be collected and saved.

TABLE 4
Study design.

| | | | | | No. of | |
|---|---|---|---|---|---|---|
| | | | Blood Collection | Tissue Collection | Animals/ | |
| Group | Dose | Treatment | and Analysis | and Analysis | time point | Time Points |
| 1 | Vehicle | Unilateral | Hematology + | ddPCR | 5 | Week 3 (Day 22 ± 3) |
| (n = 45) | | ICV P28 | ddPCR | | | Week 6 (Day 43 ± 7) |
| | | | Clinical | Histopathology | 5 | Week 12 (Day 85 ± 10) |
| | | | chemistry | | | |
| | | | coagulation | Histopathology | 5 | |
| 2 | Dose 1 | Bilateral | Hematology + | ddPCR | 5 | Week 3 (Day 22 ± 3) |
| (n = 45) | 8.0 × 10¹¹ | ICV P28 | ddPCR | | | Week 6 (Day 43 ± 7) |
| | vg | | Clinical | Histopathology | 5 | Week 12 (Day 85 ± 10) |
| | | | chemistry | | | |
| | | | coagulation | Histopathology | 5 | |
| 3 | Dose 2 | Unilateral | Hematology + | ddPCR | 5 | Week 3 (Day 22 ± 3) |
| (n = 45) | 4.0 × 10¹¹ | ICV P28 | ddPCR | | | Week 6 (Day 43 ± 7) |
| | vg | | Clinical | Histopathology | 5 | Week 12 (Day 85 ± 10) |
| | | | chemistry | | | |
| | | | coagulation | Histopathology | 5 | |
| 4 | Dose 3 | Unilateral | Hematology + | ddPCR | 5 | Week 3 (Day 22 ± 3) |
| (n = 45) | 2.0 × 10¹¹ | ICV P28 | ddPCR | | | Week 6 (Day 43 ± 7) |
| | vg | | Clinical | Histopathology | 5 | Week 12 (Day 85 ± 10) |
| | | | chemistry | | | |
| | | | coagulation | Histopathology | 5 | |
| 5 | N/A | Naïve | Hematology | Histopathology | 4 | Week 12 (Day 85 ± 10) |
| (n = 12) | | Untreated | Clinical | (n = 5) | 4 | |
| | | | chemistry | | | |
| | | | coagulation | | 4 | |

Cohort 1—Day 85±10 Termination. For Cohort 1 of this study, 60 animals designated to groups 1-4 will receive intracerebroventricular (ICV) injections on Day 1, based on their group allocation (Table 1). An additional 12 animals in Cohort 1 are part of Group 5 animals (“Naïve/untreated”, Table 1), which will not be injected. At Study Day 85±10, animals in all groups, 1-5 will be terminated, and blood and tissue samples will be collected as described in Table 5.

TABLE 5
Cohort 1 blood and tissue samples.

| | | Number | | |
|---|---|---|---|---|
| | Treatment | of | Terminal Procedure Collection | |
| Group | & Dose | Animals | Blood Analysis | Tissue Analysis |
| 1 | Vehicle | 5 | Hematology + ddPCR | ddPCR |
| | | 5 | Clinical Chemistry | Histopathology |
| | | 5 | Coagulation | |
| 2 | Dose 1 | 5 | Hematology + ddPCR | ddPCR |
| | 8.0 × 10¹¹ vg | 5 | Clinical Chemistry | Histopathology |
| | | 5 | Coagulation | |
| 3 | Dose 2 | 5 | Hematology + ddPCR | ddPCR |
| | 4.0 × 10¹¹ vg | 5 | Clinical Chemistry | Histopathology |
| | | 5 | Coagulation | |
| 4 | Dose 3 | 5 | Hematology + ddPCR | ddPCR |
| | 2.0 × 10¹¹ vg | 5 | Clinical Chemistry | Histopathology |
| | | 5 | Coagulation | |
| 5 | Untreated | 4 | Hematology | Histopathology |
| | | 4 | Clinical Chemistry | (n = 5) |
| | | 4 | Coagulation | |

Cohort 2—Day 43±7 termination. For Cohort 2, 60 animals will receive ICV injections on Day 1, based on their group allocation (Table 1). At Study Day 43±7, animals will be terminated, and blood and tissue samples will be collected as described in Table 6.

TABLE 6
Cohort 2 blood and tissue samples.

| | | Number | | |
|---|---|---|---|---|
| | Treatment | of | Terminal Procedure Collection | |
| Group | & Dose | Animals | Blood Analysis | Tissue Analysis |
| 1 | Vehicle | 5 | Hematology + ddPCR | ddPCR |
| | | 5 | Clinical Chemistry | Histopathology |
| | | 5 | Coagulation | |
| 2 | Dose 1 | 5 | Hematology + ddPCR | ddPCR |
| | 8.0 × 10¹¹ vg | 5 | Clinical Chemistry | Histopathology |
| | | 5 | Coagulation | |
| 3 | Dose 2 | 5 | Hematology + ddPCR | ddPCR |
| | 4.0 × 10¹¹ vg | 5 | Clinical Chemistry | Histopathology |
| | | 5 | Coagulation | |
| 4 | Dose 3 | 5 | Hematology + ddPCR | ddPCR |
| | 2.0 × 10¹¹ vg | 5 | Clinical Chemistry | Histopathology |
| | | 5 | Coagulation | |

Cohort 3—Day 22±3 Termination. For Cohort 3, 60 animals will receive ICV injections on Day 1, based on their group allocation (Table 1). At Study Day 22±3, animals will be terminated, and blood and tissue samples will be collected as described in Table 7.

TABLE 7
Cohort 3 blood and tissue samples.

| | | Number | Terminal Procedure Collection | |
|---|---|---|---|---|
| | Treatment | of | | Tissue |
| Group | & Dose | Animals | Blood Analysis | Analysis |
| 1 | Vehicle | 5 | Hematology + ddPCR | ddPCR |
| | | 5 | Clinical Chemistry | Histopathology |
| | | 5 | Coagulation | |
| 2 | Dose 1 | 5 | Hematology + ddPCR | ddPCR |
| | 8.0 × 10¹¹ vg | 5 | Clinical Chemistry | Histopathology |
| | | 5 | Coagulation | |
| 3 | Dose 2 | 5 | Hematology + ddPCR | ddPCR |
| | 4.0 × 10¹¹ vg | 5 | Clinical Chemistry | Histopathology |
| | | 5 | Coagulation | |
| 4 | Dose 3 | 5 | Hematology + ddPCR | ddPCR |
| | 2.0 × 10¹¹ vg | 5 | Clinical Chemistry | Histopathology |
| | | 5 | Coagulation | |

Group Assignment. Animals will be ordered and assigned to the study groups in three separate cohorts per Tables 5, 6 and 7. Each cohort of animals, including extras, up to 12 animals per group, will be weighed and assigned a numeric rank from 1 to X according to body weight in a decreasing order (the heaviest animal will be assigned rank=1). The animals will then be assigned sequentially to each study group based on termination cohort per the study design (see Tables 5, 6, 7).

Control for Bias. All attempts will be made to minimize potential study bias, including: (a) inclusion of appropriate control groups; (b) randomized assignment of animals to study groups; and (c) appropriate staggered dosing of animals across groups.

### Test Article Administration and Study Procedures

Anesthesia and Surgical Preparation. Animals are anesthetized using an inhalation anesthetic (1-5% isoflurane with 100% oxygen for induction; 1-3% for maintenance during the surgical procedures). Alternatively, animals are anesthetized with a ketamine (up to 80 mg/kg) and xylazine (up to 12 mg/kg) cocktail administered intraperitoneally.

Test Article Preparation and Delivery. All test article preparation and administration will be performed by Sponsor designated personnel. On Day 1, for each cohort (Tables 5-7), the vehicle and test article will be administered to all animals, via stereotaxic injections into ventricle, based on their group assignment. Test and control article will be kept on ice or between 2-8° C. until ready for use.

Cohort injection strategy. Injections for each termination day cohort will take place over 3-4 days. On each individual injection day, approximately 15-30 mice will be injected. In consideration of test article dose formulations, on the first day of each cohort, injections of all mice of Dose 1 will be completed, followed by vehicle injections to a subset of animals in Group 1 as time allows. On the second day of each cohort, injections of all Dose 2 mice will be completed, followed by vehicle injections to the second subset of animals as time allows for the injection team. On the third day of each cohort, injections of all Dose 3 mice will be completed, followed by vehicle injections to the third subset of animals as time allows for the injection team. If not all vehicle-injected mice have been injected across the first 3 days, the remainder of vehicle injections will take place on the fourth day.

Treatment. Treatments will be administered to animals, in accordance with Tables 5, 6 and 7, by direct injection of treatments into the lateral ventricle. Each animal, depending on dosing group, will receive either 1 unilateral injection, or 2 injections (bilateral/one injection per side) in a single surgical procedure. Surgical procedures will be performed using stereotaxic instrumentation. Injection devices, surgical instruments, drapes and gowns will be sterile where appropriate. Modifications to the procedures as described below may be performed at the discretion of the surgeon. A single dose of meloxicam (1 mg/kg, SC/IM) and/or buprenorphine (0.01-0.05 mg/kg SC) will be administered to help relieve pain from surgery after induction of anesthesia prior to skull incision. With the animal anesthetized, the skin over the cranium will be shaved (as needed) and the animal mounted in a stereotaxic frame, maintaining the animal on a nose cone for anesthesia with the head positioned by the use of ear bars and the incisor bar as applicable. Aseptic techniques will be used for all surgical procedures. The skin is disinfected with betadine solution followed by 70% alcohol wipe, or equivalent.

An approximately 2 cm incision will be made at the midline over the skull. Electrocautery may be used to achieve hemostasis of any small hemorrhages from the incision site. A Hamilton syringe with a Hamilton 2-inch, 26-gauge (or smaller), 12-degree bevel stainless steel needle will be attached to the Z-axis of the stereotactic device. The following approximate stereotaxic coordinates, based on bregma, will be used to target the lateral ventricle, either unilaterally or bilaterally:
Injection Coordinates:
   Anterior-Posterior (AP): −0.5 to −0.8 mm
   Medio-Lateral (ML): +/−1.0 mm*
   When administering unilateral injections, injection is on right side of the brain. When administering bilateral injections, first injection is on the right, second injection is on the left.
   Dorso-Ventral (DV): −2.5 mm

The actual locations of the injections may be adjusted by the surgeon as needed. If the coordinates differ from the ones described above, they will be recorded within the surgical records of the raw data.

Once needle is in desired location, syringe plunger is slowly pushed down to inject dosing solution into cranial space. The site of injection is monitored during the injection and immediately post injection to confirm quality of injection.

The desired injection volume is 8 μL per injection site. Injections will target the lateral ventricle and will include 1 or 2 total injections. The needle will be left in place for approximately 1 minute after delivering the treatment to prevent reflux. Any injection abnormalities (leakage, backflow, etc.) will be noted on the Injection sheet for the animal being injected. Following completion of injection(s) and needle removal, the incision is closed using VetBond™ surgical glue, and may be reinforced with a suture. Slight alterations in surgical procedure based on real time status of the animal may be made and will not be considered a deviation to the study protocol.

Post-Operative Care. Following the surgical procedures, animals are maintained on thermal support through recovery and will be placed in lateral recumbence alternating sides as needed. Mice are administered sterile saline subcutaneously (approximately 0.5 to 1 mL). Animals may also be provided their feed and Diet-Gel on the cage floor as supplemental feed during their surgical recovery.

### In-Life Observations and Measurements

Routine General Health Observations. Animals will be observed for changes in general appearance, behavior, and signs of illness during the acclimation period by the husbandry staff, and records will be kept on file as part of the facility records. Daily observations will be recorded beginning on Day 1 and throughout the course of the study until their designated termination day. Any animals exhibiting non-normal clinical signs prior to Day 1 will be excluded from the study.

Pre-Study Clinical Observations. All animals will receive detailed examinations for clinical signs prior to Day 1. Clinical signs indicative of poor health, stress, or other abnormalities will be noted and animals may be excluded from the study at the discretion of the Study Director/Attending Veterinarian and Sponsor Representative.

Clinical Observations. All study animals will receive a detailed clinical observation 6-10 days' post-operatively then once weekly thereafter, in conjunction with body weight, until scheduled termination. Observations noted outside of scheduled observations will be entered as unscheduled. Assessments will include, but will not be limited to: assessment of locomotor activity, neurological observations, posture, respiration, hydration status, surgical site, stereotypic behavior, bladder and bowel (stool) observations, as well as overall body condition.

The absence or presence of findings during the scheduled clinical observations will be recorded. Clinical signs indicative of poor health, stress, and pain will be noted and will be reported to the Attending Veterinarian. A final detailed examination will be conducted by the Attending Veterinarian for any animal euthanized in extremis.

Body Weights. Individual body weights will be recorded for initial group assignment shortly after animal arrival. Baseline body weights will be taken no more than 4 days prior to dosing. Body weights will be performed once weekly (coinciding with clinical observations) until scheduled termination, and a final body weight will be measured prior to scheduled termination. Per the Study Director's discretion, body weights may be measured more frequently if signs of adverse health or weight loss (i.e., greater than or equal to 10% of the previous week's weight) are observed.

Scheduled Sacrifice. Animals that survive until their scheduled termination date, Study Day 22±3, Day 43±7, Day 85±10, will be humanely euthanized, necropsied, and blood and tissue samples will be collected as described below. Animals will be euthanized in accordance with the AVMA Guidelines for Euthanasia of Animals: 2020 Edition.

### Sample/Specimen Collection

Blood Collection. Prior to necropsy, animals may be transferred to an anesthesia box and anesthetized using isoflurane (1-2%) for blood collections for hematology and droplet digital polymerase chain reaction (ddPCR), serum chemistry, and coagulation. Appropriate volume of whole blood will be collected using a 25 g needle and 1 cc syringe, or similar, from each animal via cardiac puncture, or other appropriate vessel. Blood samples will be collected from all study animals at their scheduled termination day. Blood will be processed per ASC SOPs and sent to QVL for analysis.

Samples for Hematology and ddPCR

For hematology, approximately 250-500 μL of whole blood will be placed into vials containing K₂EDTA as the anticoagulant, inverted gently several times to mix, and placed on wet ice until storage in a refrigerator set to maintain 2° C. to 8° C.

### Hematology

Collection Volume: Approximately 250-500 μL
Anticoagulant: K₂EDTA

Hematology parameters to be analyzed are provided in Table 8.

TABLE 8
Hematology parameters to be analyzed.

| | |
|---|---|
| White Blood Cell (WBC) Count | Differential White Blood |
| Differential White Blood Cell Count | Cell Count |
| | (automated and/or hand) |
| Red Blood Cell (RBC) Count | RBC and WBC Morphology |
| Hemoglobin Concentration (HGB) | Absolute Reticulocyte Count |
| Hematocrit (HCT) | Absolute Neutrophil Count |
| Mean Corpuscular Volume (MCV) | Absolute Lymphocyte Count |
| Mean Corpuscular Hemoglobin (MCH) | Absolute Monocyte Count |
| Mean Corpuscular Hemoglobin | Absolute Eosinophil Count |
| Concentration (MCHC) | |
| RBC Distribution Width (RDW) | Absolute Basophil Count |
| Platelet Count (PLT) | Absolute Large unstained |
| | cell count |
| Mean Platelet Volume (MPV) | Reticulocyte Percent |

Blood Smear

Biodistribution by Digital Droplet Polymerase Chain Reaction (ddPCR). In addition, approximately 250-500 CL of whole blood will be collected for biodistribution by ddPCR analysis. The whole blood will be placed into K₂EDTA as the anticoagulant, inverted gently several times to mix, and placed on dry ice until storage at −80° C.±10° C.

Samples for Clinical Chemistry. For clinical chemistry, approximately 500-1000 μL of whole blood will be collected into tubes containing no anticoagulant and allowed to clot at room temperature for at least 30 minutes prior to centrifugation. Clotted whole blood will be centrifuged to yield serum at a temperature of 4° C., at approximately 3000×g for 5 minutes. The serum samples will be separated following centrifugation, frozen on dry ice immediately after collection and stored frozen at −80° C.±10° C.

Serum chemistry parameters to be analyzed are provided in Table 9.

TABLE 9
Serum chemistry parameters to be analyzed.

| | |
|---|---|
| Albumin (ALB) | Calcium (CA) |
| Anion Gap | Cholesterol (CHOL) |
| Total Protein (TP) | Glucose (GLU) |
| Alkaline Phosphatase (ALP) | Inorganic Phosphorous (PHOS) |
| Alanine Aminotransferase (ALT) | Gamma glutamyl transferase (GGT) |
| Aspartate Aminotransferase (AST) | Bicarbonate (TCO2) |
| Creatine Kinase (CK) | Sodium (Na) |
| Total Bilirubin (TBil) | Potassium (K) |
| Direct Bilirubin (DBil) | Chloride (C1) |
| Indirect Bilirubin (IBil) | Globulin (Glob) |
| Urea Nitrogen (BUN) | ALB/GLOB ratio (A/G) |
| Creatine (CREAT) | BUN/CRE ratio (B/c) |

Triglycerides

Samples for Coagulation. The plasma samples will be separated following centrifugation, and stored in a freezer, set to maintain −10° C. to −30° C.

Coagulation. Collection Volume: Approximately 300-800 μL; Anticoagulant: 3.2% Sodium citrate; Processing: To plasma. Parameters Analyzed: prothrombin time (PT); activated partial thromboplastin time (APTT); fibrinogen.

Gross Necropsy. The necropsy will consist of a systematic, macroscopic external and internal examination of the animal's general physical condition and tissues (respiratory, cardiovascular, digestive and urogenital systems). This will be performed for all scheduled and unscheduled sacrifices. Any gross lesions will be documented and preserved. Details on tissue collection are described in detail below. Trained and qualified study personnel will conduct gross necropsies and tissue collection.

Tissue Collection. Tissues to be collected at termination and analyzed by either histopathology or ddPCR are presented in Table 10.

TABLE 10
Tissues to be collected and analyzed

| | | | Histopathology (Histology & | Biodistribution |
|---|---|---|---|---|
| Tissue | Weighed | Collected | Microscopic Evaluation) | by ddPCR |
| Brain | X | X | X | X |
| Dorsal Root | | X | X | |
| Ganglia | | | | |
| (Cervical Level) | | | | |
| Dorsal Root | | X | X | |
| Ganglia | | | | |
| (Thoracic Level) | | | | |
| Dorsal Root | | X | X | |
| Ganglia (Lumbar | | | | |
| Level) | | | | |
| Gross | | X | X | |
| lesions/masses | | | | |
| Heart | X | X | X | X |
| Kidney | X | X | X | X |
| Large intestine, | | X | X | |
| cecum | | | | |
| Large intestine, | | X | X | |
| colon | | | | |
| Large intestine, | | X | X | |
| rectum | | | | |
| Liver | X | X | X | X |
| Lung with | X | X | X | X |
| bronchi | | | | |
| Lymph node, | | X | X | X |
| inguinal | | | | |
| Pancreas | | X | X | X |
| Small intestine, | | X | X | |
| duodenum | | | | |
| Small intestine, | | X | X | |
| ileum | | | | |
| Small intestine, | | X | X | X |
| jejunam | | | | |
| Spinal Cord | | X | X | X |
| Spleen | X | X | X | X |
| Stomach | | X | X | |
| Testes a, b | X | X | X | X |
| Epididymides a | X | X | X | X |
| Thy mus | X | X | | |
| Urinary bladder | | X | X | |
| Muscle Tissue | | | | |
| Rib #6#7 (with | X | X | X | X |
| intercostal | | | | |
| muscle) | | | | |
| Tricepts brachii | X | X | X | X |
| Quadriceps | X | X | X | X |
| Gastrocnemius | X | X | X | X |
| Tibialis anterior | X | X | X | X |
| Extensor | X | X | X | X |
| digitorium | | | | |
| Tongue | X | X | X | X |

Tissue Collection: Histopathology. Tissues for histopathology will be collected from animals at their designated termination days per Tables 2, 3 and 4. All tissues marked in Table 6 for histopathology (except eyes), including gross lesions/abnormal tissues, will be collected and fixed in 10% neutral buffered formalin (NBF). Eyes will be fixed in Davidson Solution for 24 to 48 hours then transferred to 70% ethanol.

Tissues will be trimmed, processed, embedded in paraffin, microtomed, stained with H&E and coverslipped. When possible, muscles will be sectioned both transversely and longitudinally. The resulting slides will be microscopically quality checked. All prepared slides will be evaluated by an ACVP veterinary pathologist. A draft pathology report, consisting of tabulated microscopic data and a discussion of noteworthy changes, will be issued. Photomicrographs will be taken and annotated

ddPCR analysis. Representative samples of tissues listed in Table 10, will be collected and retained for ddPCR analysis. Specimens collected as one large portion will be placed in tubes, snap frozen in liquid nitrogen, placed in a cooler containing dry ice until placed in a freezer set to maintain −60° C.-80° C.

### Example 8: Biodistribution of INS1201-AAV9 Following Intrathecal Delivery in Nonhuman Primates (NHPs)

### Test System

Genus: Macaca
Species:fascicularis
Sex: Male
Weight: from about 2-5 kg

INS1201-AAV9 biodistribution in the periphery and skeletal muscle is measured in response to intrathecal administration.

All injections are performed intrathecally into the lumbar space of the spinal cord. Twelve (12) total animals are used, as provided in Table 11:

TABLE 11

| | Approx. | Dose (total | | | |
|---|---|---|---|---|---|
| | Weight of | vector | Test | Delivery | |
| Group | NHP | genomes (vg) | Article | method | n |
| 1 | 3 kg | 2.0 × 10¹⁴ | INS1201- | Intrathecal | 3 |
| | | | AAV9 | (IT) | |
| 2 | 3 kg | 1.0 × 10¹⁴ | INS1201- | IT | 3 |
| | | | AAV9 | | |
| 3 | 3 kg | 5.0 × 10¹³ | INS1201- | IT | 3 |
| | | | AAV9 | | |
| 4 | 3 kg | 2.5 × 10¹³ | INS1201- | IT | 3 |
| | | | AAV9 | | |

Animals selected for study will be juvenile or adult monkeys between 2 months and 5 years of age weighing approximately 3 kg. Animals are pre-screened and negative for AAV9 antibodies. Pre-screening blood collection will take place within 2 months prior to injection procedure. Animals for screening will be sedated and a blood sample collected.

Selected animals will be brought to the hospital area a few weeks prior infusion. On the day of infusion (d0), the subjects are sedated and a blood sample is collected for chemistry analysis and cell blood count (CBC). The subjects are then injected with a single dose of INS1201--AAV9 according to the dosing table above. All animals less than 9 months old will be housed with their dams. Animals older than 9 months will be housed in small groups.

The injection is performed by lumbar puncture into the subarachnoid space of the lumbar thecal sac. For intrathecal (IT) injections, subjects are placed in the lateral decubitus position and the posterior midline injection site at ˜L4/5 level identified (below the conus of the spinal cord). Under sterile conditions, a spinal needle with stylet is inserted and subarachnoid cannulation is confirmed with the flow of clear CSF from the needle, as well as injection of a small volume of iohexol followed by intra-procedural myelography. Approximately 1 ml CSF will be drained, collected and frozen as baseline sample. This is to alleviate the increase in pressure that is created by the subsequent injection of test article. To improve rostral flow distribution of the test article, the subject will then be tilted in the trendelenberg position (mild head down position). This is a routine procedure when performing CT myelograms in human subjects. For the CSF taps/IT infusions—hypodermic needles (22G ¾ or 1½″) can also be used for this purpose

In-Life Observations: Treated NHPs will be kept in isolation to reduce exposure to confounding sources of toxicity. Subjects are observed twice daily for activity, relative skin color and general health by the veterinary staff. The animals will be kept for approximately 21 days post injection. Biodistribution and clinical chemistry tests are performed on samples collected at euthanasia.

The following segments are collected from the spinal cord for biodistribution studies: cervical spinal cord, thoracic spinal cord, lumbar spinal cord, sacral spinal cord, dorsal root ganglion (DRG) root cervical level, DRG root thoracic level, DRG root lumbar level. For each spinal cord segment, two total pieces are collected. One piece is stored in 4% paraformaldehyde (PFA) and one is flash frozen.

For muscles and organs, four samples of each are collected. Two are placed in 4% PFA and two are flash frozen. Samples of the following muscles are collected: diaphragm, #6/#7 Rib with intercostal muscle and nerve, psoas muscle, deltoid, pectoralis major, biceps brachii, triceps brachii, rectus femoris, vastus medialis, vastus lateralis, gastrocnemius, tibialis anterior, soleus, tongue, masseter, extensor digitorium, rectus abdominus. Samples of the following organs are collected: heart, liver, lung, kidney, spleen.

Clinical chemistry tests are as follows: AST, ALT, GGT, Alk Phos, Potassium, Sodium, Chloride, Creatinine, Blood urea nitrogen, CBC.

Brain samples are collected as follows. The cerebellum is separated from the rest of the brain. The cerebellum is cut into four quadrants with right two quadrants stored in 4% PFA and the left two quadrants flash frozen. The brain is split into right and left hemispheres. The brain is then cut into four quadrants (coronal cuts), with the right two quadrants stored in 4% PFA and the left two quadrants flash frozen.

Six samples are taken from the quadriceps for subsequent muscle biopsy. Three are stored in 4% PFA and three are flash frozen.

Cerebral spinal fluid (CSF) and serum are also collected for biodistribution studies.

Biodistribution studies are carried out on the aforementioned samples by droplet digital polymerase chain reaction (ddPCR).

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. An intrathecal composition comprising an adeno-associated virus 9 (AAV9) particle and a pharmaceutically acceptable carrier, wherein the AAV9 particle comprises an AAV9 capsid encapsidating a vector genome, wherein the vector genome comprises from 5′ to 3′:
a 5′ inverted terminal repeat (ITR);
a MHCK7 promoter;
an SV40 intron;
a micro-dystrophin (μDys) transgene;
a SV40 poly (A) tail; and
a 3′ ITR.

2.-19. (canceled)

20. The intrathecal composition of claim 1, wherein the micro-dystrophin transgene comprises a nucleic acid sequence having at least 95% sequence identity to SEQ ID NO:5.

21. (canceled)

22. The intrathecal composition of claim 1, wherein the 5′ ITR is an AAV2 ITR.

23. The intrathecal composition of claim 22, wherein the 5′ AAV2 ITR comprises a nucleic acid sequence having at least 95% sequence identity to SEQ ID NO:1.

24. (canceled)

25. The intrathecal composition of claim 1, wherein the 3′ ITR is an AAV2 ITR.

26. The intrathecal composition of claim 25, wherein the 3′ AAV2 ITR comprises a nucleic acid sequence having at least 95% sequence identity to SEQ ID NO:7.

27.-30. (canceled)

31. The intrathecal composition of claim 1, wherein the MHCK7 promoter comprises a nucleic acid sequence having at least 95% sequence identity to SEQ ID NO:2.

32. (canceled)

33. The intrathecal composition of claim 1, wherein the SV40 intron comprises a nucleic acid sequence having at least 95% sequence identity to SEQ ID NO:4.

34. (canceled)

35. The intrathecal composition of claim 1, wherein the SV40 poly (A) tail comprises a nucleic acid sequence having at least 95% sequence identity to SEQ ID NO:6.

36.-44. (canceled)

45. The intrathecal composition of claim 1, wherein the effective amount of the AAV particle in the intrathecal composition comprises about 90% or less, or about 80% or less vector genomes than the effective amount of the AAV9 particle when present in an intravenous composition.

46. (canceled)

47. The intrathecal composition of claim 1, wherein the effective amount of the AAV9 particle comprises about 10 to 40 times less vector genomes than the effective amount of the AAV9 particle, when present in an intravenous composition.

48. A method of treating Duchenne muscular dystrophy (DMD) in a subject in need thereof, comprising, intrathecally administering to the subject, in a single dose, an effective amount of the intrathecal composition of claim 1.

49. The method of claim 48, wherein the subject is a male subject from about 6 months to about 7 years old.

50. The method of claim 48, wherein the subject is a male subject from about 2 years old to about 4 years old.

51.-61. (canceled)

62. The method of claim 48, wherein the effective amount of the intrathecal composition provides a greater therapeutic response than the identical vector genome dose of the AAV9 particle when administered intravenously.

63.-69. (canceled)

70. The method of claim 48, wherein treating comprises increasing the subject's NorthStar Ambulatory Assessment (NSAA) score subsequent to treatment, compared to a baseline NSAA score of the subject wherein the baseline NSAA score of the subject is measured prior to the subject undergoing treatment.

71. The method of claim 70, wherein increasing the score comprises increasing the score by from about 5 to about 25, from about 5 to about 20, from about 5 to about 15 or from about 5 to about 10.

72.-82. (canceled)

83. The method of claim 48, wherein treating comprises increasing the number of meters walked by the subject in the six-minute walk test (6MWT) subsequent to treatment, compared to a baseline number of meters walked by the subject in the 6MWT, wherein the baseline number of meters walked by the subject in the 6MWT is measured prior to the subject undergoing the treatment.

84.-91. (canceled)

92. The method of claim 83, wherein increasing the number of meters walked by the subject in the 6MWT comprises increasing by from about 5 meters to about 50 meters, about 5 meters to about 45 meters, about 5 meters to about 40 meters, about 5 meters to about 35 meters, about 5 meters to about 30 meters, about 5 meters to about 25 meters, about 5 meters to about 20 meters, about 5 to about 15 meters or about 5 meters to about 10 meters.

93. (canceled)

94. The method of claim 48, wherein the intrathecal composition provides greater μDys transgene expression in skeletal and cardiac muscle, compared to the amount of μDys transgene expression in liver tissue.

95.-169. (canceled)
